(19) United States
(12) Patent Application Publication
(10) Pub. No.: US 2025/0230151 A1
(43) Pub. Date: Jul. 17, 2025

(54) COMPOUNDS FOR CANCERS DRIVEN BY BRAF MUTATION

(71) Applicant: THE BOARD OF TRUSTEES OF THE UNIVERSITY OF ILLINOIS, Urbana, IL (US)

(72) Inventors: Paul J. HERGENROTHER, Champaign, IL (US); Aya Matsunaga KELLY, Urbana, IL (US)

(73) Assignee: THE BOARD OF TRUSTEES OF THE UNIVERSITY OF ILLINOIS, Urbana, IL (US)

(21) Appl. No.: 18/701,603

(22) PCT Filed: Oct. 21, 2022
(86) PCT No.: PCT/US2022/078492

§ 371 Date: Apr. 15, 2024

Related U.S. Application Data
(60) Provisional application No. 63/270,905, filed on Oct. 22, 2021.

(51) Int. Cl. C07D 417/04; A61K 31/506; A61P 35/00; C07D 417/14

(52) U.S. Cl. CPC C07D 417/04; A61K 31/506; A61P 35/00; C07D 417/14

(57) ABSTRACT

The expression of the P-glycoprotein (P-gp) efflux transporter at the blood-brain interface impedes BBB penetrance of most small molecules. Designing efflux liabilities out of compounds can be laborious and there is no generalizable approach to transform periphery-limited agents to ones active in the CNS. A target-agnostic, prospective assessment of P-gp efflux using diverse compounds indicated a reduction in molecular size or appending a carboxylic acid that enabled evasion of P-gp efflux in cell-based experiments and in mice. Such strategy was applied to transform a periphery-limited ^{V600E}BRAF inhibitor, dabrafenib, into compounds that possess potent and selective anti-cancer activity, but now also evaded P-gp-mediated efflux. When compared to dabrafenib, the compound developed herein (everafenib) has superior BBB penetrance and superior efficacy in an intracranial mouse model of metastatic melanoma, suggesting it as a lead candidate for the treatment of melanoma metastases to the brain and gliomas with BRAF mutation.

### RELATED APPLICATIONS

This application claims priority under 35 U.S.C. § 119(e) to U.S. Provisional Patent Application No. 63/270,905, filed Oct. 22, 2021, which is incorporated herein by reference.

### BACKGROUND OF THE INVENTION

Despite several decades of drug discovery efforts, developing therapeutics for brain diseases remains a formidable challenge, largely due to the blood-brain barrier (BBB). Retrospective analysis of physicochemical properties of approved drugs targeting the central nervous system (CNS) has revealed that small and lipophilic compounds have favorable BBB penetrance. These retrospective studies are, however, biased toward molecules targeting GPCRs and ion-channels, and thus trends extrapolated from these studies are not applicable to all compound types. In fact, CNS penetrating compounds that engage more contemporary targets such as kinases have shown to have starkly different properties than those of CNS approved drugs.

The general impermeability of small molecules to the brain is further compounded by high expression of P-glycoprotein (P-gp), an efflux transporter that actively pumps out many pharmaceutical agents at the BBB. P-gp inhibitors, however, generally suffer from toxicities, thus designing molecules to evade the efflux is a more attractive strategy to increase CNS penetrance. For example, crizotinib, a periphery-limited kinase inhibitor, is recognized by P-gp, so its second-generation analogue, lorlatinib, was developed to evade P-gp and exhibits improved CNS penetrance.

General and actionable strategies to design out P-gp recognition remain limited; thus, it often requires extensive medicinal chemistry campaigns with the synthesis of large numbers of compounds to reduce efflux of a substrate and discover a P-gp non-substrate and BBB penetrant derivative.

Accordingly, a need remains for improved CNS drugs that are not substates for P-gp.

### SUMMARY

Herein is describe the results from efforts to elucidate properties of P-gp recognition via prospective analysis of a diverse set of compounds. Reduction of molecular weight (MW) or installation of a carboxylic acid can, in many cases, facilitate P-gp efflux evasion in cell-based systems and in mouse models. These findings were applied to redesign a BRAF inhibitor, leading to a potent version that has reduced efflux propensity, enhanced brain penetration, and activity in a challenging intracranial mouse model of melanoma. The strategies described herein served as a guide to reducing P-gp-mediated efflux and, together with existing approaches to improve brain accumulation, facilitated the conversion of non-CNS-penetrant drugs into ones that have enhanced accumulation and activity in the CNS.

Accordingly, this disclosure provides a compound of Formula I:

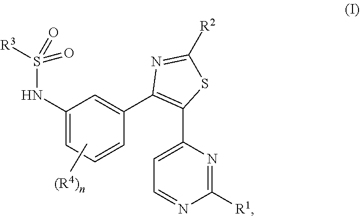

or a salt thereof,
wherein
R¹ is —NR^{a}R^{b}, —(C₁-C₆)alkyl-J¹, or —(C₃-C₆)cycloalkyl-J²;
   R^{a} and R^{b} are each independently H, —(C₁-C₆)alkyl-J³, —(C₃-C₆)cycloalkyl-J⁴, or phenyl-J⁵;
   J¹, J², J³, J⁴, and J⁵ are each independently CO₂H or tetrazol-2-yl;
R² is —(C₁-C₆)alkyl or —(C₃-C₆)cycloalkyl;
R³ is —(C₁-C₆)alkyl, —(C₃-C₆)cycloalkyl, or phenyl(R^{c})ₘ;
   each R^{c} is independently halo or —(C₁-C₆)alkyl;
   m is 0, 1,2,3,4, or 5;
each R⁴ is independently halo or —(C₁-C₆)alkyl; and
n is 2, 1, 3, 4, or 0;
   wherein each alkyl moiety is independently branched or unbranched, and optionally substituted. Examples of the substituents that can be employed to provide the substituents on substituted alkyl moieties are described below in the definition of the term substituent.

This disclosure also provides a method for method for treatment of cancer comprising, administering to a subject in need of cancer treatment a therapeutically effective amount of a compound of Formula I as described herein, wherein the compound has a permeability glycoprotein (P-gp) efflux ratio of about 3 or less, or 2 or less.

The invention provides novel compounds of any one of Formulas I-V, intermediates for the synthesis of compounds of any one of Formulas I-V, as well as methods of preparing compounds of any one of Formulas I-V. The invention also provides compounds of any one of Formulas I-V that are useful as intermediates for the synthesis of other useful compounds. The invention provides for the use of compounds of any one of Formulas I-V for the manufacture of medicaments useful for the treatment of bacterial infections in a mammal, such as a human.

The invention provides for the use of the compositions described herein for use in medical therapy. The medical therapy can be treating cancer, for example, brain cancer, breast cancer, endometrial cancer, liver cancer, lung cancer, pancreatic cancer, prostate cancer, colon cancer, or melanoma. The invention also provides for the use of a composition as described herein for the manufacture of a medicament to treat a disease in a mammal, for example, cancer in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. (A) Everafenib displays similar potency to dabrafenib, while everafenib-CO₂H is superior to vemurafenib (Table 3, FIG. 2A), and both maintain cytotoxicity selective to cell lines harboring the ^{V600E}BRAF mutation across different tissue origins in a 72 h cell viability assay. Data are plotted as mean±s.e.m., n=3 biological replicates. (B) Everafenib and everafenib-CO₂H inhibit MAPK signaling, suggesting retention of the mode of action in a cell line harboring ^{V600E}BRAF mutation (A375) but not WT (CHL-1). Phospho-MEK1/2 and phospho-ERK1/2 inhibition by DMSO, 10 μM PLX4720, 1 μM dabrafenib, 1 μM encorafenib, 1 μM everafenib, and 10 M everafenib-CO₂H for 1 h. Higher concentrations of PLX4720 and everafenib-CO₂H were chosen due to their higher IC₅₀ value. (C) Dose-response of phospho-ERK1/2 inhibition in A375 cells treated with DMSO; 0.01, 0.1, 1, and 10 μM of everafenib or everafenib-CO₂H; and 10 μM of dabrafenib or PLX4720 for 1 and 24 h.

FIG. 2. (A) Everafenib-CO₂H is more potent than vemurafenib while maintaining cytotoxicity selective to cell lines harboring ^{V600E}BRAF mutation across different tissue origins in a 72-hr cell viability assay. Everafenib-CO₂H and vemurafenib were evaluated at higher concentrations due to their higher IC₅₀ values. Data are plotted as mean±s.e.m., n=3 biological replicates. (B) Everafenib and everafenib-CO₂H inhibit MAPK signaling in AM-38 cells, suggesting retention of mode of action in AM-38 with ^{V600E}BRAF mutation. Phospho-MEK1/2 and phospho-ERK1/2 inhibition by DMSO, 10 μM PLX4720, 1 μM dabrafenib, 1 μM encorafenib, 1 μM everafenib and 10 μM everafenib-CO₂H for 1 hr. (C) Dose-dependent phospho-ERK1/2 inhibition in AM38 cells treated with DMSO, 0.01, 0.1, 1 and 10 μM of everafenib and 10 μM of dabrafenib for 1 hr.

FIG. 3. Everafenib is BBB-penetrant and efficacious in the A375 intracranial model. (A,B) Everafenib is more BBB-penetrant than dabrafenib. Brain-to-serum ratios (A) and brain concentration (B) of BRAF inhibitors at 60 min, following a single 25 mg/kg IV injection. Everafenib achieves the highest brain partitioning. Number of mice per cohort n>3. Statistical significance of each compound compared to everafenib was determined by using a two-sample Student's t-test (two-tailed test, assuming equal variance). (C) Evaluation of everafenib in an intracranial mouse model. 50,000 A375 melanoma cells were intracranially implanted into 7-week old female athymic nude mice. Five days after inoculation, mice were treated with the vehicle or 50 mg/kg of dabrafenib or everafenib intraperitoneal (IP) once a day for 5 days. After 2 days off, another five daily doses were administered, a total of 10 treatments in the model. Cells were tested to be free of pathogens prior to inoculation. Vehicle vs dabrafenib: P=0.009, vehicle vs everafenib: P=0.005, dabrafenib versus everafenib: P=0.0018. Number of mice per cohort=6. Statistical significance was determined by a two-tailed log-rank (Mantel-Cox) test. **P<0.01 ***P<0.001 ****P<0.0001.

FIG. 4. Biodistribution data of dabrafenib, everafenib-CO₂H, and everafenib. Serum and brain concentrations and brain-to-serum ratios of dabrafenib (A-C), everafenib-CO₂H (D-F) and everafenib (G-I). Mice were treated with 2.5 mg/kg elacridar or its vehicle intravenously; after 30 min, mice were administered with a single injection of 25 mg/kg compound via lateral tail vein. Number of mice per cohort n>3. Statistical significance was determined by using a two-sample Student's t-test (two-tailed test, assuming equal variance). * P<0.05, ** P<0.01, *** P<0.001.

FIG. 5. Biodistribution data of BRAF inhibitors. Serum (A) and brain (B) concentration and brain-to-serum ratios (C) of dabrafenib, encorafenib, PLX4720, dabrafenib, everafenib-CO₂H and everafenib after 15 and 60 min following a single intravenous injection of 25 mg/kg of drugs. Number of mice per cohort n>3. Statistical significance was determined by using a two-sample Student's t-test (two-tailed test, assuming equal variance). * P<0.05, ** P<0.01, *** P<0.001, **** P<0.0001.

FIG. 6. Pharmacokinetics analysis of everafenib (Table 4).

### DETAILED DESCRIPTION

To assess chemical features that influence P-gp efflux in a target-agnostic fashion, a collection of structurally diverse compounds was required. Commercially available drug—like compounds (including kinase inhibitors) were utilized, and these were complemented by a collection of natural product-like compounds. Produced using the “complexity-to-diversity” (CtD) strategy, this collection includes scores of compounds with structures distinct from those in traditional screening sets and CNS drugs, and members were systematically modified as needed during follow-up experiments.

Major findings from the target-agnostic evaluation of P-gp efflux are that (1) reducing MW or (2) appending a carboxylic acid can reduce P-gp-mediated efflux. The observations of MW dependence are consistent with other studies reporting high-MW compounds as strong P-gp binders and efflux substrates. The second observation that a majority of carboxylic acid-containing compounds evade P- gp efflux is surprising, and indeed, the use of a carboxylic acid to enhance CNS penetrance is counterintuitive. However, observations in the literature that carboxylic acid moieties are detrimental to overall CNS penetrance appear to predominately be due to reduction in permeability of anionic compounds.

Compound design strategies based on the major findings have provided new compounds with reduced efflux liability toward P-gp. Substructures of vemurafenib, encorafenib and dabrafenib were combined to yield the hybrid compounds everafenib and everafenib-CO₂H (Chart 1).

### Chart 1. Design of Hybrid BRAF Inhibitors.

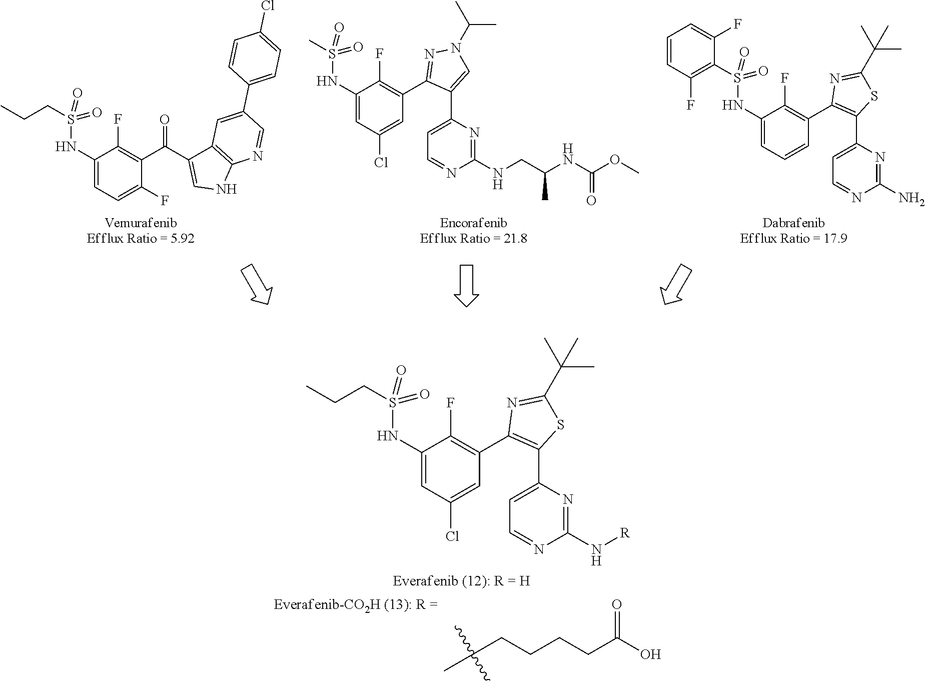

Additional information and data supporting the invention can be found in the following publication by the inventors: J. Am. Chem. Soc. 2022, 144, 12367-12380 and its Supporting Information, which is incorporated herein by reference in its entirety.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R.J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated.

Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, The terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms.

The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modem Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl(iso-propyl), 1-butyl, 2-methyl-1-propyl(isobutyl), 2-butyl(sec-butyl), 2-methyl-2-propyl(t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at two different carbon atoms.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF3, OCF3, R′, 0, S, C(O), S(0), methylenedioxy, ethylenedioxy, N(R′)₂, SR′, SOR′, SO2R′, SO2N(R′)₂, SO3R′, C(O)R′, C(O)C(O)R′, C(O)CH2C(O)R′, C(S)R′, C(O)OR′, OC(O)R′, C(O)N(R′)₂, OC(O)N(R′)₂, C(S)N(R′)₂, (CH2)0-2NHC(O)R′, N(R′)N(R′)C(O)R′, N(R′)N(R′)C(O)OR′, N(R′)N(R′)CON(R′)₂, N(R′)SO2R′, N(R′)SO2N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C1-C6)alkyl), and wherein the carbon-based moiety can itself be further substituted. When a substituent is monovalent, such as, for example, F or Cl, it is bonded to the atom it is substituting by a single bond. When a substituent is divalent, such as 0, it is bonded to the atom it is substituting by a double bond; for example, a carbon atom substituted with 0 forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The terms “racemic mixture” and “racemate” refer to an equimolar mixture of two enantiomeric species, devoid of optical activity.

The term “enantiomerically enriched” (“ee”) as used herein refers to mixtures that have one enantiomer present to a greater extent than another. Reactions that provide one enantiomer present to a greater extent than another would therefore be “enantioselective” (or demonstrate “enantioselectivity”). In one embodiment of the invention, the term “enantiomerically enriched” refers to a mixture having at least about 2% ee; in another embodiment of the invention, the term “enantiomerically enriched” refers to a mixture having at least about 5% ee; in another embodiment of the invention, the term “enantiomerically enriched” refers to a mixture having at least about 20%; in another embodiment of the invention, the term “enantiomerically enriched” refers to a mixture having at least about 50%; in another embodiment of the invention, the term “enantiomerically enriched” refers to a mixture having at least about 80%; in another embodiment of the invention, the term “enantiomerically enriched” refers to a mixture having at least about 90%; in another embodiment of the invention, the term “enantiomerically enriched” refers to a mixture having at least about 95%; in another embodiment of the invention, the term “enantiomerically enriched” refers to a mixture having at least about 98%; in another embodiment of the invention, the term “enantiomerically enriched” refers to a mixture having at least about 99%. The term “enantiomerically enriched” includes enantiomerically pure mixtures which are mixtures that are substantially free of the species of the opposite optical activity or one enantiomer is present in very low quantities, for example, 0.01%, 0.001% or 0.0001%.

The term “IC₅₀” is generally defined as the concentration required to inhibit a specific biological or biochemical function by half, or to kill 50% of the cells in a designated time period, typically 24 hours.

Alternate identifiers are shown in parenthesis for the following compounds:
compound 6-83 (Dabrafenib-CO₂H, 11); compound 6-261 (Everafenib, 12); and compound 6-263 (Everafenib-CO₂H, 13).

### Embodiments of the Technology

This disclosure provides a compound of Formula I:

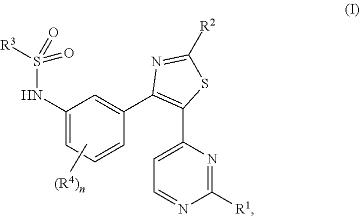

or a salt thereof,
wherein
R¹ is —NR^{a}R^{b}, —(C₁-C₆)alkyl-J¹, or —(C₃-C₆)cycloalkyl-J²;
   R^{a} is H, —(C₁-C₆)alkyl-J³, —(C₃-C₆)cycloalkyl-J⁴, or phenyl-J⁵;
   R^{b} is H, —(C₁-C₆)alkyl-J³, —(C₃-C₆)cycloalkyl-J⁴, or phenyl-J⁵;
   J¹, J², J³, J⁴, and J^{P} are each independently CO₂H, SO₂OH, PO(OH)₂, or tetrazol-2-yl;
R² is —(C₁-C₆)alkyl or —(C₃-C₆)cycloalkyl;
R³ is —(C₁-C₆)alkyl, —(C₃-C₆)cycloalkyl, or phenyl(R^{c});
   each R^{c} is independently halo or —(C₁-C₆)alkyl;
   m is 0, 1,2,3,4, or 5;
each R⁴ is independently halo or —(C₁-C₆)alkyl; and
n is 2, 1, 3, 4, or 0;
   wherein each alkyl moiety is independently branched or unbranched, and optionally substituted.

In some embodiments, the compound of Formula I is not dabrafenib; R³ is not 2,6-difluorophenyl when R¹ is NH₂, R² is tert-butyl, R⁴ is 2-fluoro, and n is 1; R³ is not 2,5-difluorophenyl when R¹ is NH₂, R² is tert-butyl, R⁴ is 5-chloro and 2-fluoro, and n is 2; and R³ is not n-propyl when R¹ is NH₂, R² is tert-butyl, R⁴ is 2,5-chloro, and n is 2.

In various embodiments, R¹ is —NHR^{b} or —(C₁-C₆)alkyl-J¹. In various embodiments, R¹ is: NH₂,

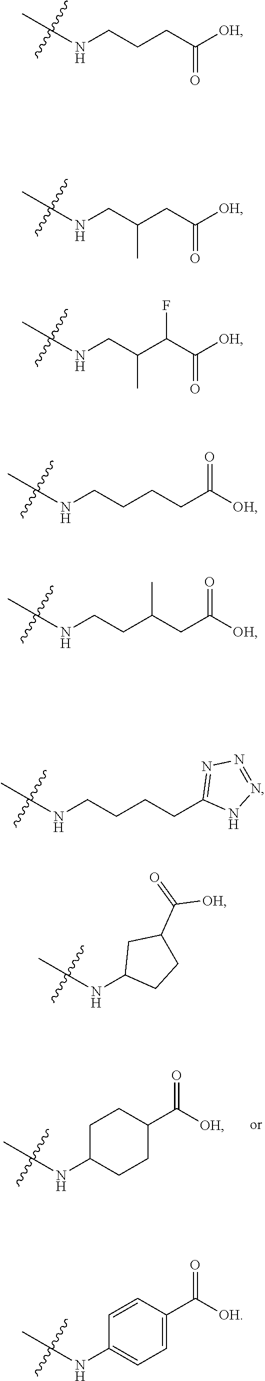

In various embodiments, R¹ is:

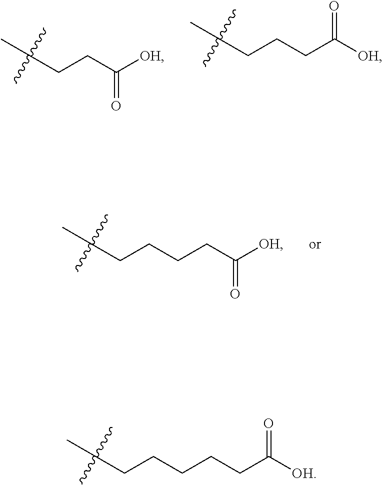 [SMILES: *CCCC(=O)O ; *CCC(=O)O ; *CCCCC(=O)O ; *CCCCCC(=O)O]

In various embodiments, R² is tert-butyl or —C(CH₃)₂CO₂H. In various embodiments, R³ is propyl, butyl, pentyl,

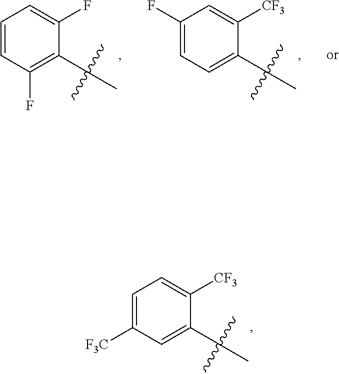 [SMILES: *c1c(F)cccc1F ; *c1ccc(F)cc1C(F)(F)F ; *c1cc(C(F)(F)F)ccc1C(F)(F)F]

wherein additionally, R¹ is optionally NH₂ in these embodiments.

In various embodiments, R² is n-propyl, isopropyl, n-butyl, isobutyl, tert-butyl, n-pentyl, or isopentyl. In various embodiments, the moiety (C₁-C₆)alkyl is (C₂-C₆)alkyl, (C₃-C₆)alkyl, or (C₄-C₆)alkyl. In various embodiments, the moiety (C₁-C₆)alkyl is methyl, ethyl, propyl, butyl, pentyl, or hexyl. In various embodiments, the moiety (C₁-C₆)alkyl or (C₂-C₆)alkyl has optionally one or more substitutions. In various embodiments, the moiety (C₃-C₆)cycloalkyl is cyclopropyl, cyclobutyl, cyclopentyl, or cyclohexyl. In various embodiments, the moiety (C₃-C₆)cycloalkyl or (C₄-C₆)cycloalkyl has optionally one or more substitutions.

In various embodiments, at least one R⁴ is fluoro or 2-fluoro. In various embodiments, at least one R⁴ is chloro or 5-chloro. In various embodiments, one R⁴ is fluoro, another R⁴ is chloro, and n is 2. In various embodiments, one R⁴ is 2-fluoro, another R⁴ is 5-chloro, and n is 2. In various embodiments, one R⁴ is in the 2-position, 4-position, 5-position, or 6-position. In various embodiments, a second R⁴ is in the 2-position, 4-position, 5-position, or 6-position. In some embodiments, a third R⁴ is in the 2-position, 4-position, 5-position, or 6-position. In some embodiments, a fourth R⁴ is in the 2-position, 4-position, 5-position, or 6-position.

In various embodiments, the compound is represented by Formula II:

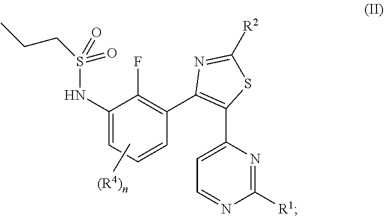

In various embodiments, the compound is represented by Formula III:

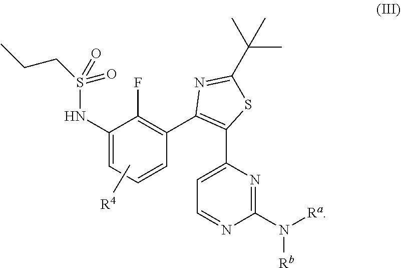

In various embodiments, the compound is represented by Formula IV:

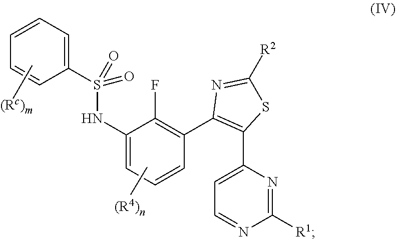

wherein n is 0, 1, 2, or 3.

In various embodiments, the compound is represented by Formula V:

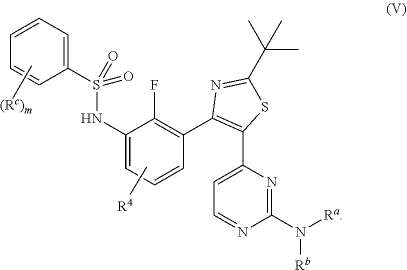

In various embodiments, R^{a} is H; and R^{b} is: H,

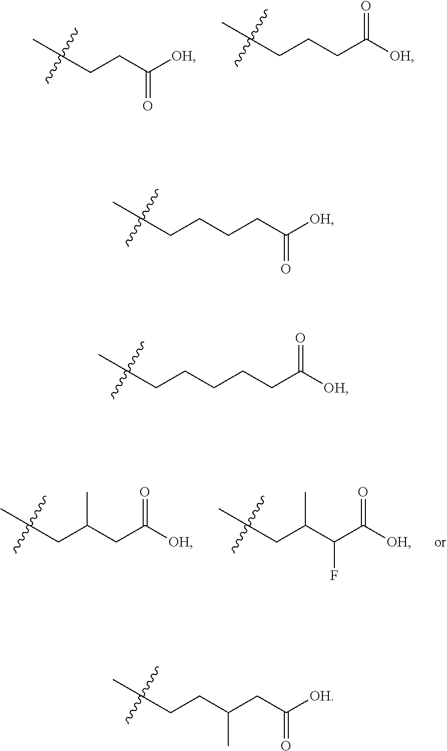

In various embodiments, R¹ is —NH(C₁-C₆)alkyl-CO₂H or —(C₁-C₆)alkyl-CO₂H.

In some embodiments, the compound is 6-261, or alternatively, the compound is 6-263.

In some embodiments, the compound is the (R)-enantiomer. In other embodiments, the compound is the (S)-enantiomer. In some embodiments, the compound rotates polarized light dextrorotatory. In other embodiments, the compound rotates polarized light levorotatory.

In some embodiments, the compound is:

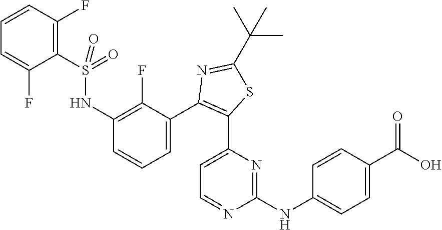 [SMILES: CC(C)(C)c1nc(-c2cccc(NS(=O)(=O)c3c(F)cccc3F)c2F)c(-c2ccnc(Nc3ccc(C(=O)O)cc3)n2)s1]

4-((4-(2-(tert-butyl)-4-(3-((2,6-difluorophenyl)sulfonamido)-2-fluorophenyl)thiazol-5-yl)pyrimidin-2-yl)amino)benzoic acid (6-77),

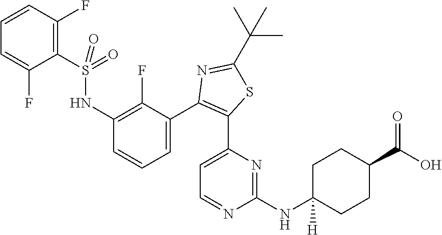 [SMILES: CC(C)(C)c1nc(-c2cccc(NS(=O)(=O)c3c(F)cccc3F)c2F)c(-c2ccnc(N[C@H]3CC[C@@H](C(=O)O)CC3)n2)s1]

(1s,4s)-4-((4-(2-(tert-butyl)-4-(3-((2,6-difluorophenyl)sulfonamido)-2-fluorophenyl)thiazol-5-yl)pyrimidin-2-yl)amino)cyclohexane-1-carboxylic acid (6-85),

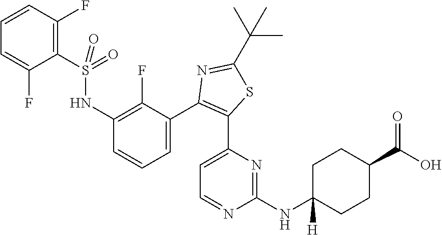 [SMILES: CC(C)(C)c1nc(-c2cccc(NS(=O)(=O)c3c(F)cccc3F)c2F)c(-c2ccnc(N[C@H]3CC[C@H](C(=O)O)CC3)n2)s1]

(1r,4r)-4-((4-(2-(tert-butyl)-4-(3-((2,6-difluorophenyl)sulfonamido)-2-fluorophenyl)thiazol-5-yl)pyrimidin-2-yl)amino)cyclohexane-1-carboxylic acid (6-89),

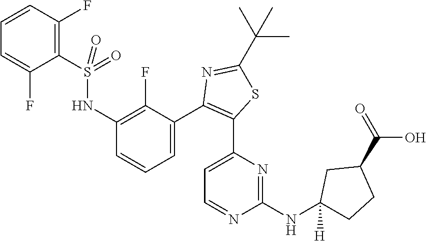 [SMILES: CC(C)(C)c1nc(-c2cccc(NS(=O)(=O)c3c(F)cccc3F)c2F)c(-c2ccnc(N[C@@H]3CC[C@H](C(=O)O)C3)n2)s1]

(1S,3R)-3-((4-(2-(tert-butyl)-4-(3-((2,6-difluorophenyl)sulfonamido)-2-fluorophenyl)thiazol-5-yl)pyrimidin-2-yl)amino)cyclopentane-1-carboxylic acid (6-91),

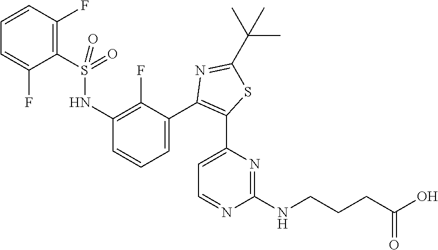 [SMILES: CC(C)(C)c1nc(-c2cccc(NS(=O)(=O)c3c(F)cccc3F)c2F)c(-c2ccnc(NCCCC(=O)O)n2)s1]

4-((4-(2-(tert-butyl)-4-(3-((2,6-difluorophenyl)sulfonamido)-2-fluorophenyl)thiazol-5-yl)pyrimidin-2-yl)amino)butanoic acid (6-86),

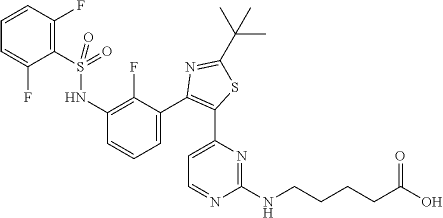 [SMILES: CC(C)(C)c1nc(-c2cccc(NS(=O)(=O)c3c(F)cccc3F)c2F)c(-c2ccnc(NCCCCC(=O)O)n2)s1]

5-((4-(2-(tert-butyl)-4-(3-((2,6-difluorophenyl)sulfonamido)-2-fluorophenyl)thiazol-5-yl)pyrimidin-2-yl)amino)pentanoic acid (6-83),

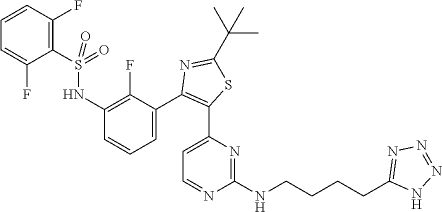 [SMILES: CC(C)(C)c1nc(-c2cccc(NS(=O)(=O)c3c(F)cccc3F)c2F)c(-c2ccnc(NCCCCc3nnn[nH]3)n2)s1]

N-(3-(5-(2-((4-(1H-tetrazol-5-yl)butyl)amino)pyrimidin-4-yl)-2-(tert-butyl)thiazol-4-yl)-2-fluorophenyl)-2,6-difluorobenzenesulfonamide (6-121),

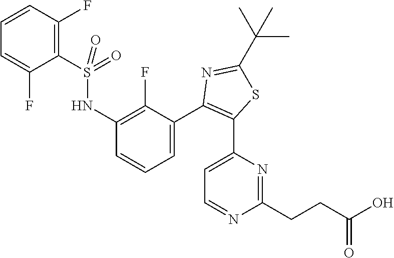 [SMILES: CC(C)(C)c1nc(-c2cccc(NS(=O)(=O)c3c(F)cccc3F)c2F)c(-c2ccnc(CCC(=O)O)n2)s1]

3-(4-(2-(tert-butyl)-4-(3-((2,6-difluorophenyl)sulfonamido)-2-fluorophenyl)thiazol-5-yl)pyrimidin-2-yl)propanoic acid (6-97),

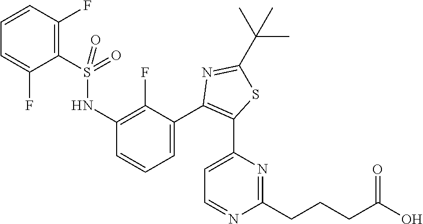 [SMILES: CC(C)(C)c1nc(-c2cccc(NS(=O)(=O)c3c(F)cccc3F)c2F)c(-c2ccnc(CCCC(=O)O)n2)s1]

4-(4-(2-(tert-butyl)-4-(3-((2,6-difluorophenyl)sulfonamido)-2-fluorophenyl)thiazol-5-yl)pyrimidin-2-yl)butanoic acid (6-191),

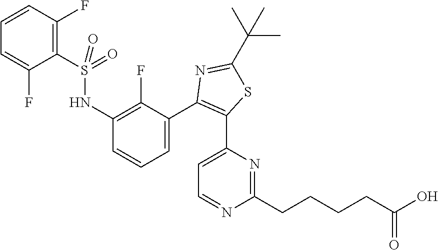 [SMILES: CC(C)(C)c1nc(-c2cccc(NS(=O)(=O)c3c(F)cccc3F)c2F)c(-c2ccnc(CCCCC(=O)O)n2)s1]

5-(4-(2-(tert-butyl)-4-(3-((2,6-difluorophenyl)sulfonamido)-2-fluorophenyl)thiazol-5-yl)pyrimidin-2-yl)pentanoic acid (6-131),

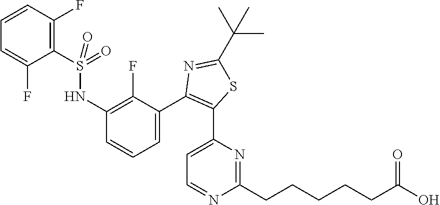 [SMILES: CC(C)(C)c1nc(-c2cccc(NS(=O)(=O)c3c(F)cccc3F)c2F)c(-c2ccnc(CCCCCC(=O)O)n2)s1]

6-(4-(2-(tert-butyl)-4-(3-((2,6-difluorophenyl)sulfonamido)-2-fluorophenyl)thiazol-5-yl)pyrimidin-2-yl)hexanoic acid (6-193),

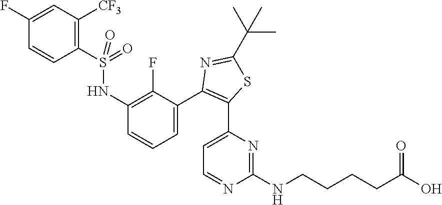 [SMILES: CC(C)(C)c1nc(-c2cccc(NS(=O)(=O)c3ccc(F)cc3C(F)(F)F)c2F)c(-c2ccnc(NCCCCC(=O)O)n2)s1]

5-((4-(2-(tert-butyl)-4-(2-fluoro-3-((4-fluoro-2-(trifluoromethyl)phenyl)sulfonamido)phenyl) thiazol-yl)pyrimidin-2-yl)amino)pentanoic acid (6-181),

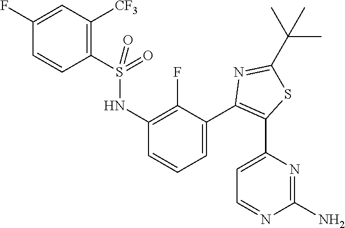 [SMILES: CC(C)(C)c1nc(-c2cccc(NS(=O)(=O)c3ccc(F)cc3C(F)(F)F)c2F)c(-c2ccnc(N)n2)s1]

N-(3-(5-(2-aminopyrimidin-4-yl)-2-(tert-butyl)thiazol-4-yl)-2-fluorophenyl)-4-fluoro-2-(trifluoromethyl)benzenesulfonamide (6-166),

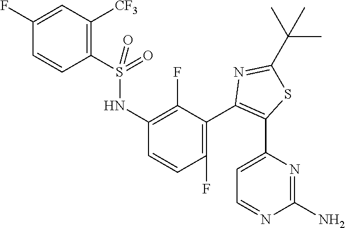 [SMILES: CC(C)(C)c1nc(-c2c(F)ccc(NS(=O)(=O)c3ccc(F)cc3C(F)(F)F)c2F)c(-c2ccnc(N)n2)s1]

N-(3-(5-(2-aminopyrimidin-4-yl)-2-(tert-butyl)thiazol-4-yl)-2,4-difluorophenyl)-4-fluoro-2-(trifluoromethyl)benzenesulfonamide (6-167),

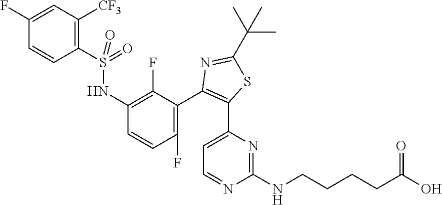 [SMILES: CC(C)(C)c1nc(-c2c(F)ccc(NS(=O)(=O)c3ccc(F)cc3C(F)(F)F)c2F)c(-c2ccnc(NCCCCC(=O)O)n2)s1]

5-((4-(2-(tert-butyl)-4-(2,6-difluoro-3-((4-fluoro-2-(trifluoromethyl)phenyl)sulfonamido)phenyl) thiazol-5-yl)pyrimidin-2-yl)amino)pentanoic acid (6-179),

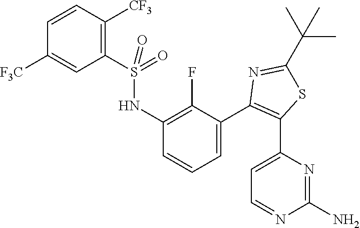 [SMILES: CC(C)(C)c1nc(-c2cccc(NS(=O)(=O)c3cc(C(F)(F)F)ccc3C(F)(F)F)c2F)c(-c2ccnc(N)n2)s1]

N-(3-(5-(2-aminopyrimidin-4-yl)-2-(tert-butyl)thiazol-4-yl)-2-fluorophenyl)-2,5-bis(trifluoromethyl)benzenesulfonamide (6-163),

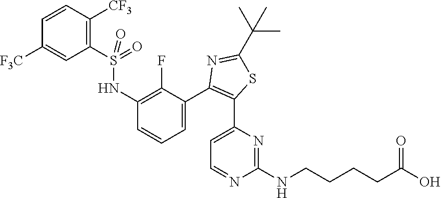 [SMILES: CC(C)(C)c1nc(-c2cccc(NS(=O)(=O)c3cc(C(F)(F)F)ccc3C(F)(F)F)c2F)c(-c2ccnc(NCCCCC(=O)O)n2)s1]

5-((4-(4-(3-((2,5-bis(trifluoromethyl)phenyl)sulfonamido)-2-fluorophenyl)-2-(tert-butyl)thiazol-5-yl)pyrimidin-2-yl)amino)pentanoic acid (6-173),

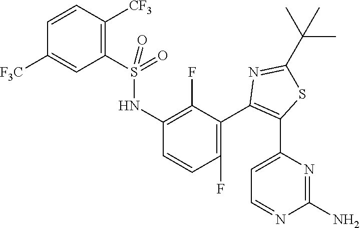 [SMILES: CC(C)(C)c1nc(-c2c(F)ccc(NS(=O)(=O)c3cc(C(F)(F)F)ccc3C(F)(F)F)c2F)c(-c2ccnc(N)n2)s1]

N-(3-(5-(2-aminopyrimidin-4-yl)-2-(tert-butyl)thiazol-4-yl)-2,4-difluorophenyl)-2,5-bis(trifluoromethyl)benzenesulfonamide (6-145),

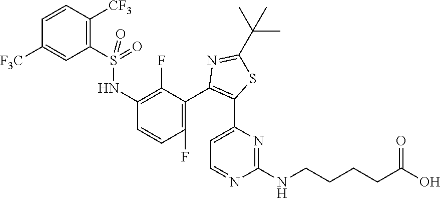 [SMILES: CC(C)(C)c1nc(-c2c(F)ccc(NS(=O)(=O)c3cc(C(F)(F)F)ccc3C(F)(F)F)c2F)c(-c2ccnc(NCCCCC(=O)O)n2)s1]

5-((4-(4-(3-((2,5-bis(trifluoromethyl)phenyl)sulfonamido)-2,6-difluorophenyl)-2-(tert-butyl)thiazol-5-yl)pyrimidin-2-yl)amino)pentanoic acid (6-150),

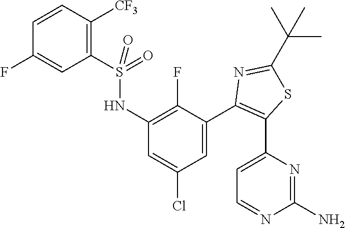 [SMILES: CC(C)(C)c1nc(-c2cc(Cl)cc(NS(=O)(=O)c3cc(F)ccc3C(F)(F)F)c2F)c(-c2ccnc(N)n2)s1]

N-(3-(5-(2-aminopyrimidin-4-yl)-2-(tert-butyl)thiazol-4-yl)-5-chloro-2-fluorophenyl)-5-fluoro-2-methylbenzenesulfonamide (6-244),

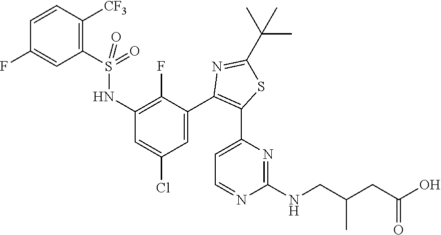 [SMILES: CC(CNc1nccc(-c2sc(C(C)(C)C)nc2-c2cc(Cl)cc(NS(=O)(=O)c3cc(F)ccc3C(F)(F)F)c2F)n1)CC(=O)O]

4-((4-(2-(tert-butyl)-4-(5-chloro-2-fluoro-3-((5-fluoro-2-methylphenyl)sulfonamido)phenyl) thiazol-5-yl)pyrimidin-2-yl)amino)-3-methylbutanoic acid (6-251),

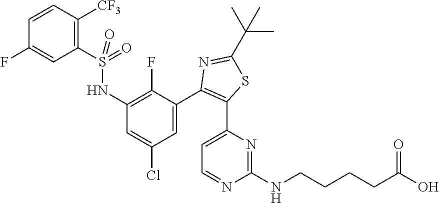 [SMILES: CC(C)(C)c1nc(-c2cc(Cl)cc(NS(=O)(=O)c3cc(F)ccc3C(F)(F)F)c2F)c(-c2ccnc(NCCCCC(=O)O)n2)s1]

5-((4-(2-(tert-butyl)-4-(5-chloro-2-fluoro-3-((5-fluoro-2-methylphenyl)sulfonamido)phenyl) thiazol-5-yl)pyrimidin-2-yl)amino)pentanoic acid (6-249),

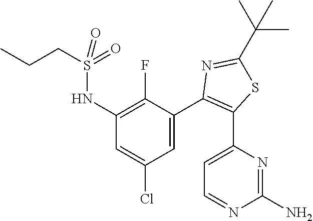 [SMILES: CCCS(=O)(=O)Nc1cc(Cl)cc(-c2nc(C(C)(C)C)sc2-c2ccnc(N)n2)c1F]

N-(3(-(2-aminopyrimidin-4-yl)-2-(tert-butyl)thiazol-4-yl)-5-chloro-2-fluorophenyl)propane-1-sulfonamide (6-261), 6

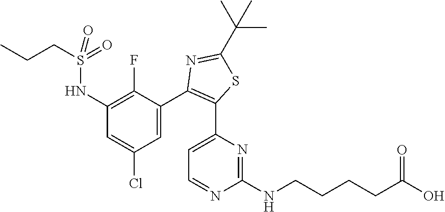 [SMILES: CCCS(=O)(=O)Nc1cc(Cl)cc(-c2nc(C(C)(C)C)sc2-c2ccnc(NCCCCC(=O)O)n2)c1F]

5-((4-(2-(tert-butyl)-4-(5-chloro-2-fluoro-3-(propylsulfonamido)phenyl)thiazol-5-yl)pyrimidin-2-yl)amino)pentanoic acid (6-263),

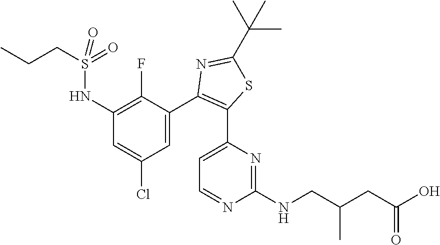 [SMILES: CCCS(=O)(=O)Nc1cc(Cl)cc(-c2nc(C(C)(C)C)sc2-c2ccnc(NCC(C)CC(=O)O)n2)c1F]

4-((4-(2-(tert-butyl)-4-(5-chloro-2-fluoro-3-(propylsulfonamido)phenyl)thiazol-5-yl)pyrimidin-2-yl)amino)-3-methylbutanoic acid (6-265),

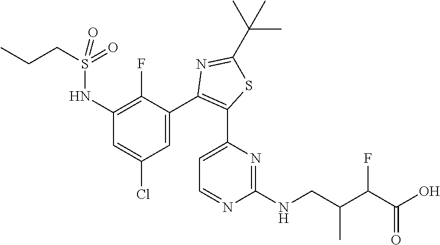 [SMILES: CCCS(=O)(=O)Nc1cc(Cl)cc(-c2nc(C(C)(C)C)sc2-c2ccnc(NCC(C)C(F)C(=O)O)n2)c1F]

4-((4-(2-(tert-butyl)-4-(5-chloro-2-fluoro-3-(propylsulfonamido)phenyl)thiazol-5-yl)pyrimidin-2-yl)amino)-2-fluoro-3-methylbutanoic acid (8-41),

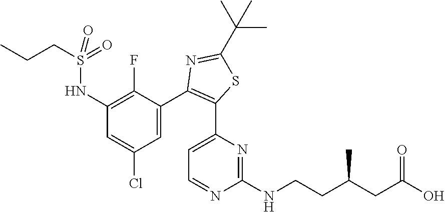 [SMILES: CCCS(=O)(=O)Nc1cc(Cl)cc(-c2nc(C(C)(C)C)sc2-c2ccnc(NCC[C@@H](C)CC(=O)O)n2)c1F]

(R)-5-((4-(2-(tert-butyl)-4-(5-chloro-2-fluoro-3-(propylsulfonamido)phenyl)thiazol-5-yl)pyrimidin-2-yl)amino)-3-methylpentanoic acid (8-43),

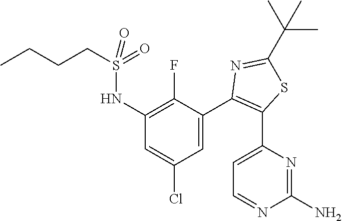 [SMILES: CCCCS(=O)(=O)Nc1cc(Cl)cc(-c2nc(C(C)(C)C)sc2-c2ccnc(N)n2)c1F]

N-(3-(5-(2-aminopyrimidin-4-yl)-2-(tert-butyl)thiazol-4-yl)-5-chloro-2-fluorophenyl)butane-1-sulfonamide,

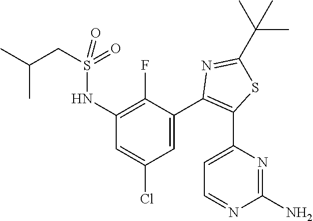 [SMILES: CC(C)CS(=O)(=O)Nc1cc(Cl)cc(-c2nc(C(C)(C)C)sc2-c2ccnc(N)n2)c1F]

N-(3-(5-(2-aminopyrimidin-4-yl)-2-(tert-butyl)thiazol-4-yl)-5-chloro-2-fluorophenyl)-2-methylpropane-1-sulfonamide, or an enantiomer or diastereomer thereof.

Also, this disclosure provides a composition or combination comprising a compound disclosed herein and a pharmaceutically acceptable excipient.

Additionally, this disclosure provides a method for treatment of a cancer comprising administering to a subject in need of cancer treatment an effective amount of a compound of a formula disclosed herein. In various embodiments, the cancer harbors the ^{V600E}BRAF mutation.

In various embodiments, the compound is an inhibitor of the ^{V600E}BRAF enzyme. In various embodiments, the compound has a permeability glycoprotein (P-gp) efflux ratio of about 5, about 4.5, about 4, about 3.5, about 3, about 2.5, about 2, about 1.5, about 1, about 0.9, about 0.8, about 0.7, about 0.6, about 0.5, about 0.4, about 0.3, about 0.2, about 0.1, or less than 1. In various embodiments, compound has a P-gp efflux ratio of 1.0±0.75.

In various embodiments, the compound has a brain to serum ratio of about 0.25, about 0.5, about 0.75, about 1, about 1.25, about 1.5, about 1.75, about 2, about 2.5, about 3, about 3.5, about 4, about 4.5, or about 5 or more. In various embodiments, the compound and a second agent are simultaneously or sequentially administered to the subject for the treatment of the cancer. In some embodiments, a composition or combination of the compound and one or more second agents are formulated in a manner to provide a medicament wherein the compound and each second agent can be administered separately to a subject. In various embodiments, a combination of the compound and the second agent have synergistic anti-cancer activity. In various embodiments, the cancer is brain cancer, lung cancer, colon cancer, breast cancer, endometrial cancer, liver cancer, or melanoma.

The compounds disclosed in U.S. Patent Publication No. 2013/0096149 are incorporated herein by reference and the compounds therein may be excluded from one or more embodiments of this invention.

### Results and Discussion

Design of ^{V600E}BRAF Inhibitors that Evade P-gp Efflux. The twin observations about lowering MW and adding carboxylic acids has aided in the conversion of drugs that are periphery-limited (due to high P-gp effiux) to those that evade effiux and hence were active in the CNS. For addition of a carboxylic acid, ideal candidates selected for this conversion to a BBB-penetrant version were compounds that are highly permeable yet limited by P-gp effiux. Anti-proliferative targeted kinase inhibitors, such as dabrafenib and imatinib (with limited CNS exposure due to P-gp efflux), were of particular interest, as BBB-penetrant versions are highly sought for treatment of metastatic lesions in the brain. Considering that kinase inhibitors exist outside of the traditional CNS-targeted small-molecule chemotypes, the ability to rationally redesign such compounds would be a valuable and directly actionable feature of the twin observations. Dabrafenib (Scheme 1) has marked potency and selectivity toward melanoma cells harboring the ^{V600E}BRAF mutation and was approved to treat peripheral melanoma in 2013; however, dabrafenib is strongly recognized by P-gp and thus unable to effectively accumulate in the brain. When assessed in mice lacking P-gp, dabrafenib reaches therapeutically relevant concentrations in the brain. Dabrafenib is efficacious in intracranial tumors in mice when co-treated with a membrane permeabilizer but has minimal activity on its own, suggesting that a BBB-penetrant version could be highly efficacious.

The wealth of structure-activity relationship (SAR) data established in the development of BRAF inhibitors informed a design of derivatives incorporating reduced MW or a carboxylic acid. Disclosed SAR information on over 290 derivatives and the co-crystal structure of dabrafenib bound to human ^{V600E}BRAF enzyme revealed that the aminopyrimidine tail group (substitution of the 2 position of the pyrimidine ring, see Scheme 1) is amenable to modification, and polar functional groups could be incorporated without compromising selective anti-cancer activity. These considerations guided the design of three derivatives containing carboxylic acid moieties on the aminopyrimidine tail (compound 11 in Scheme 1 and others in Table 1). Evaluation of these carboxylic acid-functionalized dabrafenib derivatives in a transwell assay revealed a significant reduction in efflux ratio (ER); while dabrafenib was readily expelled with an ER of 17.9, the new compounds had ER values less than 2.0 (Scheme 1 and Table 1). Evaluation of their ability to induce death of A375 melanoma cells (which harbor ^{V600E}BRAF) revealed compound 11 as the most potent with an IC₅₀ of 115 nM (Table 1).

Scheme 1. Conversion of dabrafenib to versions that evade P-gp efflux. Structures of dabrafenib, everafenib, and everafenib-CO₂H, along with their permeabilities and ERs as assessed in the MDR1-MDCK transwell assay.

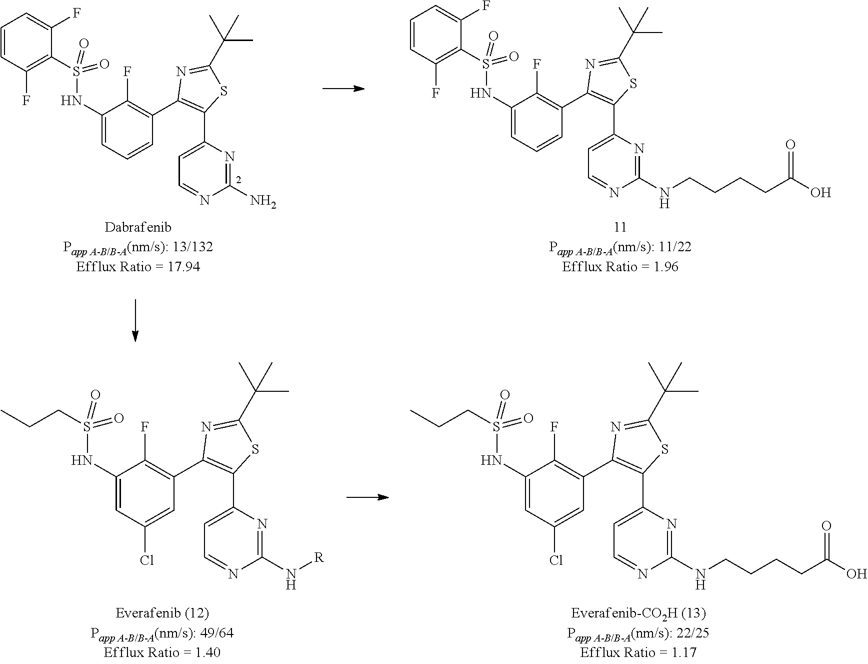

With this success in hand and with the goal of creating more potent compounds that evade P-gp efflux through reduction in MW or addition of a carboxylic acid, examination of the three approved drugs targeting ^{V600E}BRAF inspired the design of hybrid compounds (see Chart 1). These novel compounds incorporate the propyl sulfonamide from vemurafenib (for reduction in MW), the 5-chloro-2-fluoro substitution pattern of the phenyl core from encorafenib (to increase lipophilicity), the tert-butyl thiazole from dabrafenib, and 2,4-pyrimidine from dabrafenib and encorafenib. This led to the hybrid compound 12, hereafter referred to as everafenib (Scheme 1), a neutral compound with an MW that has been reduced below 500 g/mol (484.01 g/mol relative to 519.56 g/mol for dabrafenib), as well as its carboxylic acid-containing version 13, everafenib-CO₂H (Scheme 1). After the synthesis of these new compounds, their assessment in the transwell assay validated the design strategy and revealed that both these compounds indeed have low ERs: everafenib has an ER of 1.40, and everafenib-CO₂H has an ER of 1.17 (Scheme 1). Encorafenib, a structurally similar approved BRAF inhibitor with a non-carboxylic acid-containing side chain, is strongly recognized by P-gp with an ER of 21.8 (Chart 1).

TABLE 1
Design of dabrafenib derivatives and evaluation of cytotoxicity and efflux. Structure of
dabrafenib and its derivatives modified with a carboxylic acid moiety. Below each compound is their
respective 72-hr IC₅₀ value against A375 cells, and permeability and efflux ratios assessed in the
MDR1-MDCK transwell assay. Data are plotted as mean ± s.e.m., n = 3 biological replicates.

| 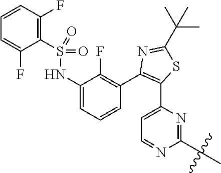 [SMILES: *c1nccc(-c2sc(C(C)(C)C)nc2-c2cccc(NS(=O)(=O)c3c(F)cccc3F)c2F)n1] | 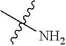 [SMILES: *N] | 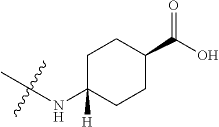 [SMILES: *N[C@H]1CC[C@H](C(=O)O)CC1] | 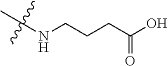 [SMILES: *NCCCC(=O)O] | 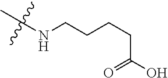 [SMILES: *NCCCCC(=O)O] |
|---|---|---|---|---|
| Compound | Dabrafenib | 41 (6-89) | 42 (6-86) | 11 (6-83) |
| A375 IC₅₀ (nM) | 7.46 ± 1.10 | 166 ± 4.09 | 628 ± 105 | 115 ± 9.85 |
| P_{appA-B} (nm/s) | 12.6 ± 6.72 | 22.4 ± 1.26 | 8.44 ± 2.14 | 10.9 ± 0.97 |
| P_{appB-A} (nm/s) | 132 ± 16.3 | 12.5 ± 4.36 | 4.45 ± 0.40 | 21.7 ± 3.71 |
| ER | 17.94 ± 6.97 | 0.56 ± 0.18 | 0.61 ± 0.16 | 1.96 ± 0.22 |

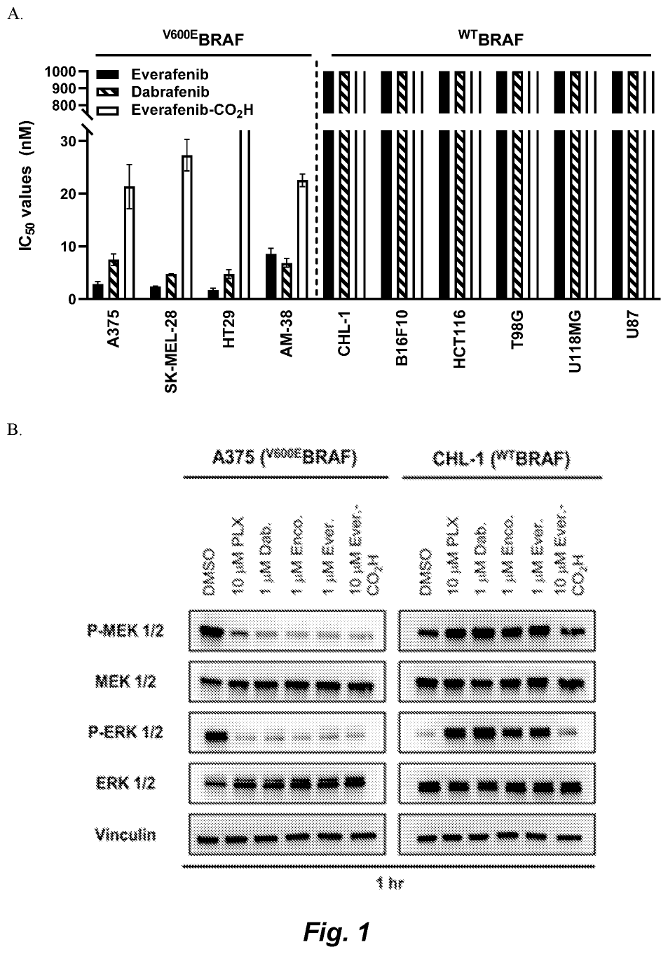

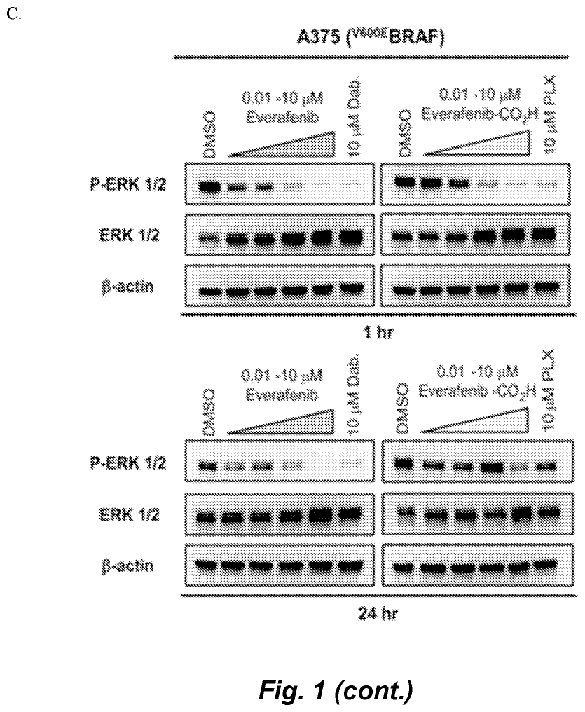

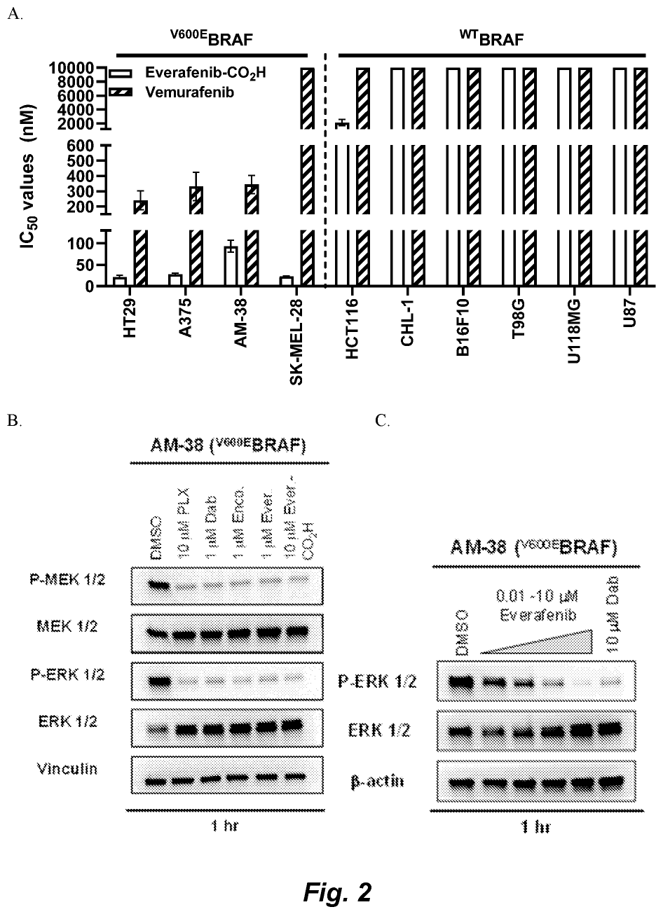

Evaluation against cancer cells in culture revealed that everafenib is a highly potent inducer of death as assessed against a panel of ^{V600E}BRAF melanoma cell lines with IC₅₀ values of 2-10 nM, comparable to or better than dabrafenib (FIG. 1A and Table 2, 3). Everafenib-CO₂H is also potent against cancer cells with the ^{V600E}BRAF mutation, although not as potent as everafenib (FIG. 1A) but still markedly more potent than vemurafenib (Table 3, FIG. 2A). Critically, both everafenib and everafenib-CO₂H maintain their selectivity for cancer cells harboring ^{V600E}BRAF mutation, with IC₅₀ values greater than 1 μM in cell lines with ^{WT}BRAF (FIG. 1A, Table 3 and FIG. 2A).

TABLE 2
Dabrafenib derivatives retain the mode of action of dabrafenib. Structures of everafenib and
everafenib-CO₂H. Below each compound is their respective 72-hour IC₅₀ value against A375 cells,
and permeability and efflux ratios assessed in the MDR1-MDCK transwell assay.

| | 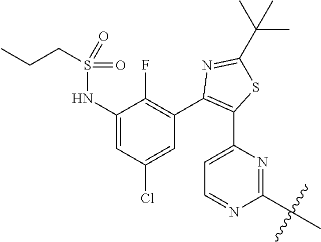 [SMILES: *c1nccc(-c2sc(C(C)(C)C)nc2-c2cc(Cl)cc(NS(=O)(=O)CCC)c2F)n1] | 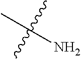 [SMILES: *N] | 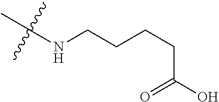 [SMILES: *NCCCCC(=O)O] |
|---|---|---|---|
| Compound | Dabrafenib | Everafenib (12) | Everafenib-CO₂H (13) |
| A375 IC₅₀ (nM) | 7.46 ± 1.10 | 2.80 ± 0.53 | 27.3 ± 3.01 |
| P_{appA-B} (nm/s) | 12.6 ± 6.72 | 49.2 ± 8.06 | 21.5 ± 0.70 |
| P_{appB-A} (nm/s) | 132 ± 16.3 | 64.2 ± 24.5 | 24.9 ± 4.35 |
| ER | 17.9 ± 6.97 | 1.40 ± 0.65 | 1.17 ± 0.22 |

To further assess if the cytotoxicity of everafenib and everafenib-CO₂H is derived from inhibition of the constitutively activated MAPK signaling found in ^{V600E}BRAF cells, MEK1/2 and ERK1/2 phosphorylation was assessed. The MAPK pro-growth signaling was strongly inhibited upon treatment with everafenib and everafenib-CO₂H after 1 h of treatment, comparable to dabrafenib, encorafenib, and PLX4720 (a progenitor compound to vemurafenib) in A375 cells (FIG. 11B) and AM-38 glioma cells with ^{V600E} BRAF (FIG. 29).

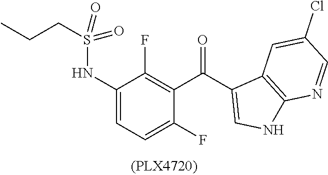 [SMILES: CCCS(=O)(=O)Nc1ccc(F)c(C(=O)c2c[nH]c3ncc(Cl)cc23)c1F]

TABLE 3
Everafenib: similar potency to dabrafenib and
superior to everafenib-CO₂H and vemurafenib.

| | | | | | Everafenib- |
|---|---|---|---|---|---|
| | IC₅₀ (nM) | Dabrafenib | Everafenib (12) | Vemurafenib | CO₂H (13) |
| ^{V600E}BRAF | A375 | 7.46 ± 1.10 | 2.80 ± 0.53 | 331 ± 93 | 27.3 ± 3.01 |
| | HT29 | 4.74 ± 0.84 | 1.67 ± 0.36 | 240 ± 61.7 | 21.4 ± 4.21 |
| | AM-38 | 6.84 ± 0.84 | 8.50 ± 1.2 | 344 ± 59.3 | 93.2 ± 14.1 |
| | SK-MEL-28 | 4.76 ± 0.42 | 2.35 ± 0.13 | 47% dead at 10 μM | 22.5 ± 1.20 |
| ^{WT}BRAF | HCT116 | >1000 | >1000 | >10000 | 2100 ± 456 |
| | CHL-1 | >1000 | >1000 | >10000 | >10000 |
| | B16F10 | >1000 | >1000 | >10000 | >10000 |
| | T98G | >1000 | >1000 | >10000 | >10000 |
| | U118MG | >1000 | >1000 | >10000 | >10000 |
| | U87 | >1000 | >1000 | >10000 | >10000 |

Following the identical treatment in CHL-1, a ^{WT}BRAF melanoma cell line, inhibition of the MAPK signaling was not observed (FIG. 1B). This is consistent with previous reports where vemurafenib does not inhibit the MAPK signaling but paradoxically activates ERK1/2 phosphorylation in a ^{WT}BRAF cell line. After both 1 and 24 h of treatment in A375 cells, there was a clear dose-dependent inhibition imparted by everafenib and everafenib-CO₂H (FIG. 1C, see FIG. 1C for AM-38). Inhibition of ERK1/2 phosphorylation was sustained even after 24 h at the highest concentrations of everafenib and everafenib-CO₂H, while dabrafenib or PLX4720 had diminished inhibition at this time point (FIG. 1C).

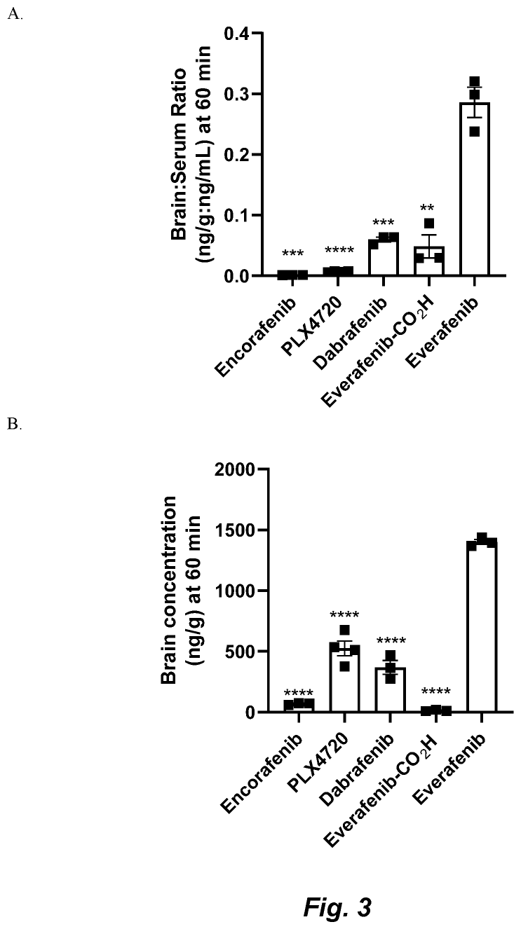

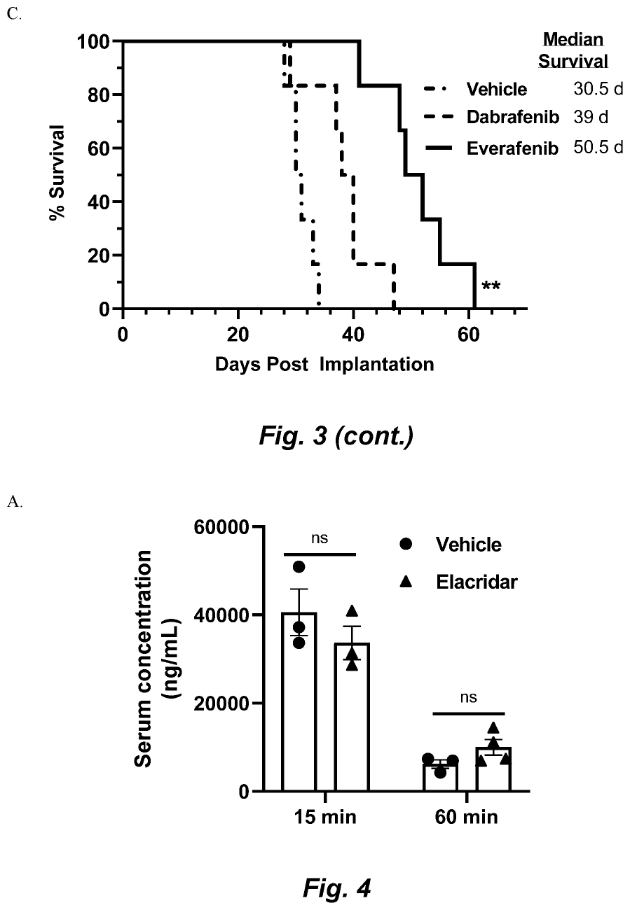

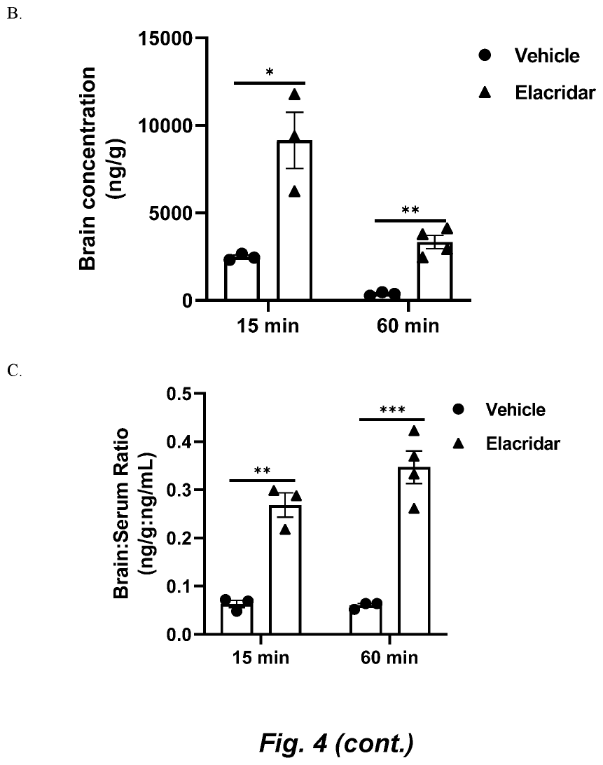

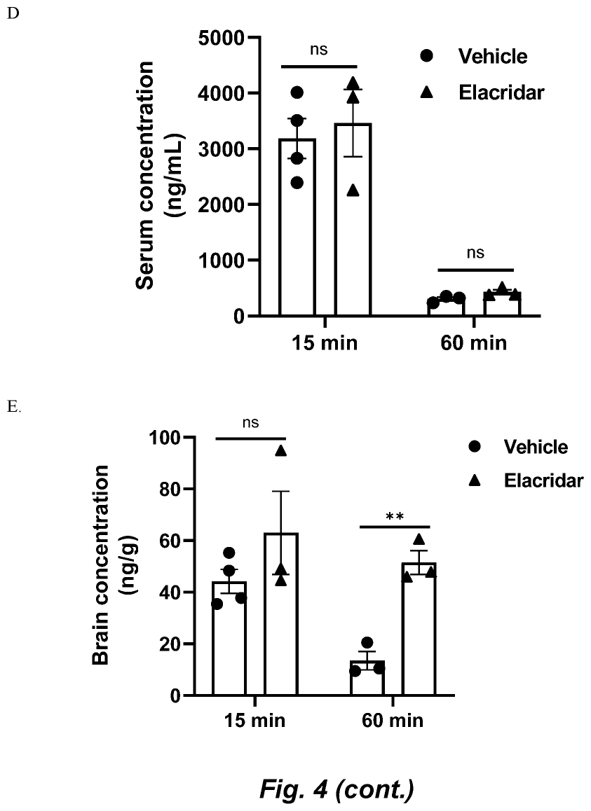

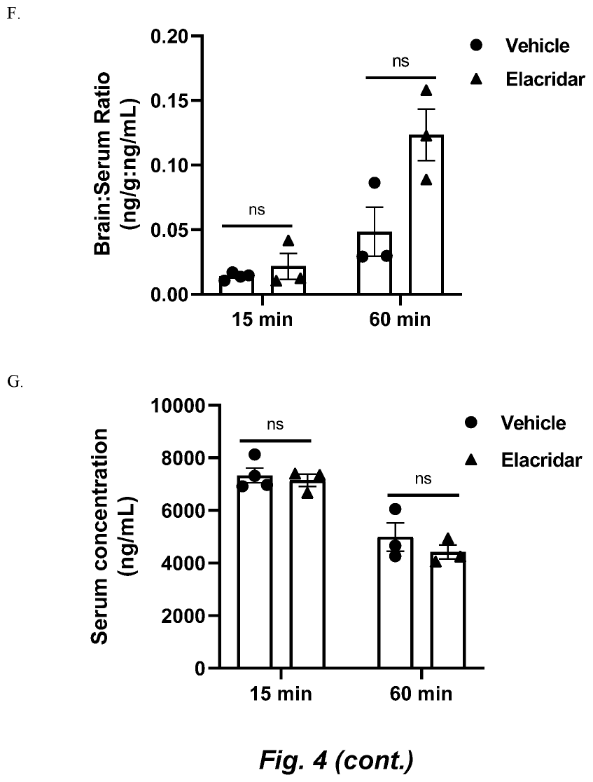

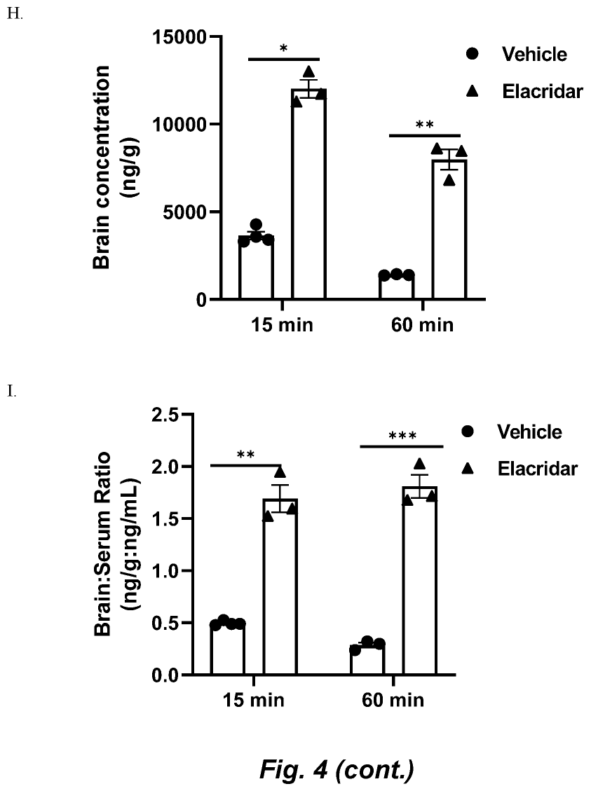

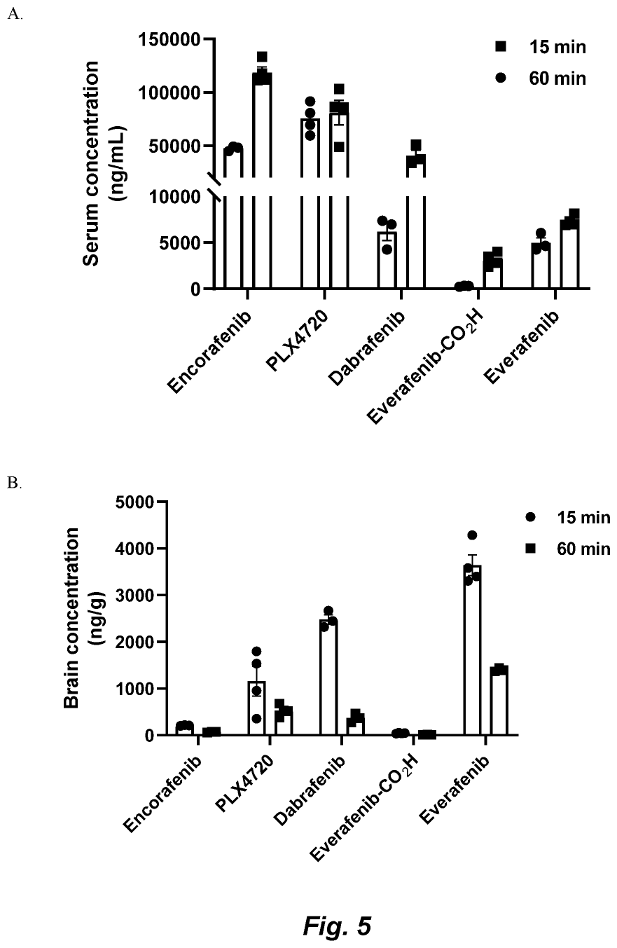

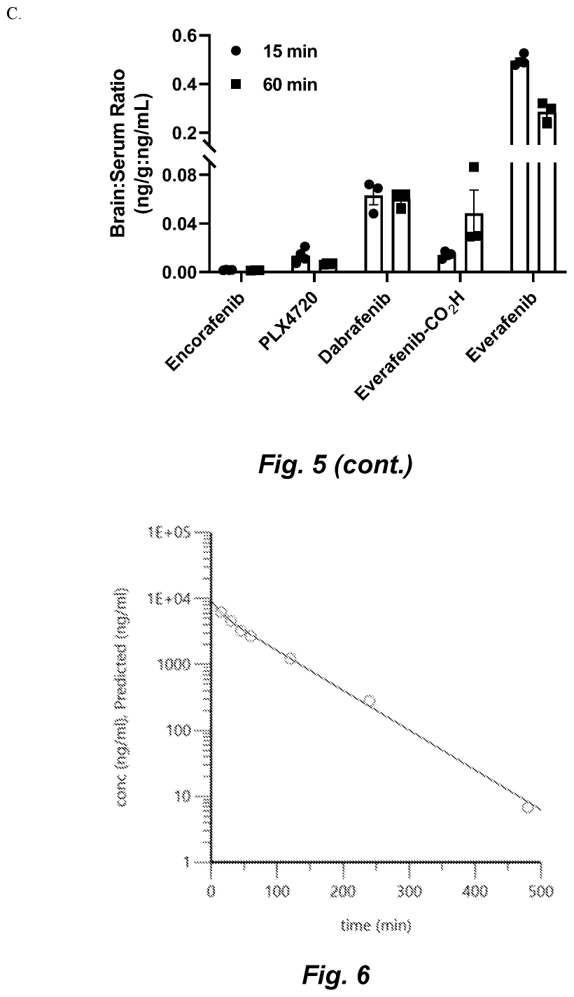

Everafenib is BBB-Penetrant and Has Efficacy in an Intracranial Model of Metastatic Melanoma. Given their low efflux liabilities and potent anti-cancer efficacy in cell culture, the BBB penetrance of everafenib and everafenib-CO₂H in mice was evaluated and compared alongside approved agents targeting ^{V600E}BRAF. The cell culture permeabilities of everafenib (49-64 nm/s) and everafenib-CO₂H (22-25 nm/s) (Scheme 1) suggested that everafenib may be the preferred candidate in vivo. Indeed, assessment of brain and serum drug levels 60 min following a single intravenous injection of encorafenib, PLX4720, dabrafenib, or everafenib-CO₂H revealed that all these compounds have poor brain-to-serum ratios (0.0014-0.0069 ng/g:ng/mL, FIG. 3A). Significantly, everafenib achieved a markedly higher brain-to-serum ratio than all other inhibitors (0.286 ng/g:ng/mL, FIG. 3A). Most importantly, the absolute brain concentration of everafenib was substantially higher than that of dabrafenib and other BRAF inhibitors (FIG. 3B). Evaluating dabrafenib and everafenib-CO₂H in the P-gp in vivo assay revealed that P-gp inhibition greatly enhanced the BBB penetrance of dabrafenib 4.3- and 5.8-fold at 15 and 60 min (FIG. 4A-C). Consistent with the results from the cell-based efflux assay, everafenib-CO₂H had slight but not statistically significant potentiation of 1.5- and 2.5-fold (FIG. 4D-F). As shown in FIG. 3B, everafenib achieves an absolute concentration in the brain markedly higher than that of dabrafenib, although it did display potentiation with elacridar (FIG. 4G-I), suggesting that other efflux mechanisms (beyond P-gp) may be operational in vivo or that murine and human P-gp recognize this compound differently. All in vivo biodistribution data of BRAF inhibitors (serum and brain concentration and brain-to-serum ratios of multiple time points) are found in FIG. 4A-I and FIG. 5A-C.

The increased BBB penetrance observed for everafenib relative to dabrafenib, in addition to its pharmacokinetic assessment in mice (FIG. 6 and Table 4), its outstanding potency and selectivity in cell culture (FIG. 1A), and sustained inhibition of ERK1/2 phosphorylation (FIG. 1C), suggested this compound as an excellent candidate for evaluation in a mouse model of melanoma metastasis to the brain. Such a model has been developed using intracranial implantation of the A375 melanoma cell line, and thus, this macro-metastatic model via injection of A375 cells to the mouse forebrain was used to assess everafenib and dabrafenib head-to-head. In the experiment, mice were intracranially implanted with A375 cells and then 5 days later were treated with 50 mg/kg of dabrafenib or everafenib (two cycles of once-per-day for 5 days via IP injection, a total of 10 doses). Mice treated with everafenib outperformed dabrafenib and increased median survival from 39 to 50.5 days (FIG. 3C). This result suggests that the increased BBB penetrance of everafenib, attributed to reduced P-gp efflux, improved its efficacy compared to dabrafenib.

TABLE 4
CD-1 mice were treated with 20 mg/kg everafenib via IP injection,
with three mice per time point (0, 15, 30, 45, 60, 120, 240,
480, and 960 min). At the indicated time points, mice were
sacrificed, and blood were collected. The serum concentration
of everafenib was determined by LC-MS/MS. Data are shown
as mean of three biological replicates. The pharmacokinetic
parameters were calculated with a one-compartment model using
a nonlinear regression program (Phoenix WinNonlin Version
8.1, Certara USA Inc., Princeton, NJ 08540 USA).

| Parameter | Units | Estimate |
|---|---|---|
| AUC | min*ng/ml | 503964.6 |
| T_{1/2} | min | 37.3 |
| Cmax | ng/ml | 9364.8 |
| CL | ml/min/kg | 39.7 |
| Vss | ml/kg | 2692.9 |

### CONCLUSIONS

Actionable twin observations from an unbiased analysis discussed above enabled the development of two potent and selective ^{V600E}BRAF inhibitors with reduced efflux liabilities. Following the discovery of the V600E mutation in BRAF as an oncogenic driver in 50% of melanomas, the development and approval of vemurafenib, followed by dabrafenib and encorafenib, led to dramatic improvement of the survival outcome of melanoma patients whose tumors harbored that mutation. However, over 50% of metastatic melanoma patients eventually develop metastasis to the CNS. Despite undetectable and/or low cerebrospinal fluid levels in patients and limited penetrance to the brain in preclinical models, there are several reports on the use of dabrafenib and vemurafenib in melanoma patients with brain metastatic lesions. When combined with trametinib (a MEK inhibitor and also a P-gp substrate), dabrafenib provides only a short duration (6.5 months) of intracranial response in clinical trials. This modest activity is attributed to limited brain accumulation and highlights the need for novel ^{V600E}BRAF inhibitors that have significantly enhanced BBB penetrance. The brain accumulation of the top compound detailed herein, everafenib, supersedes those of all approved agents targeting ^{V600E}BRAF in head-to-head experiments, suggesting its potential for superior efficacy against intracranial tumors. In the A375 intracranial mouse model, everafenib is indeed superior to dabrafenib, presumably due to its potent activity, sustained phospho-ERK1/2 inhibition, and the lack of P-gp efflux.

Beyond metastatic melanoma, dabrafenib and vemurafenib have some efficacy in primary brain cancer patients with the ^{V600E}BRAF mutation. In a histology agnostic trial with vemurafenib, four out of seven pleomorphic xanthoastrocytoma patients responded to ^{V600E}BRAF inhibition, and it also had meaningful clinical activity in pediatric low-grade glioma patients with ^{V600E}BRAF. In cell culture studies, everafenib exhibited comparable cytotoxicity to dabrafenib against AM-38, a glioblastoma cell line with ^{V600E}BRAF, while having no activity against glioma cell-lines with ^{WT}BRAF (U118MG, T98G, U87). These data suggest that a BRAF inhibitor with superior CNS penetrance may provide greater survival benefits for patients with primary brain cancers whose tumors harbor this mutation. More generally, the observations herein about MW and the presence of a carboxylic acid complement existing tools to improve BBB penetrance of compounds and, in conjunction with carboxylic acid isosteres that maintain their anionic nature, should aid the design of BBB-penetrant versions of drugs from a variety of classes.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, a-ketoglutarate, and P-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.10% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use.

The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), 4,820,508 (Wortzman), 4,608,392 (Jacquet et al.), and 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

The compounds described herein can be effective anti-tumor agents and have higher potency and/or reduced toxicity as compared dabrafenib, vemurafenib, or encorafenib. Preferably, compounds of the invention are more potent and less toxic than dabrafenib, vemurafenib, or encorafenib, and/or avoid a potential site of catabolic metabolism encountered with dabrafenib, vemurafenib, or encorafenib, i.e., have a different metabolic profile than dabrafenib, vemurafenib, or encorafenib.

The invention provides therapeutic methods of treating cancer in a subject such as a mammal, which involve administering to a mammal having cancer an effective amount of a compound or composition described herein. A mammal includes a primate, human, rodent, canine, feline, bovine, ovine, equine, swine, caprine, bovine and the like. Cancer refers to any various type of malignant neoplasm, for example, colon cancer, breast cancer, melanoma and leukemia, and in general is characterized by an undesirable cellular proliferation, e.g., unregulated growth, lack of differentiation, local tissue invasion, and metastasis.

The ability of a compound of the invention to treat cancer may be determined by using assays well known to the art. For example, the design of treatment protocols, toxicity evaluation, data analysis, quantification of tumor cell kill, and the biological significance of the use of transplantable tumor screens are known.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Experimental design, assay validation

A novel compound collection composed of complex and diverse compounds derived from natural products, termed “Complexity-to-Diversity Collection” was employed. Distinct from traditional screening sets, this diverse compound collection enabled an unbiased target-agnostic analysis of biological transport, in this case P-gp mediated efflux. The CtD compounds were evaluated by the efflux pump and their permeability using the P-gp transwell assay using adapted and optimized known protocols. Compound transport across epithelial cells overexpressing P-gp from basolateral-to-apical and apical-to-basolateral directions were quantified to calculate permeability and efflux ratio. The optimized assay was validated using a series of controls, both CNS-penetrant (P-gp non-substrate) and periphery-limited (P-gp substrate) drug-like and natural product-like small-molecules, as well as co-administration with the P-gp inhibitor, elacridar.

Unbiased assessment of P-gp efflux. For the initial test set, 87 compounds were chosen, however, there was no clear trend of efflux recognition with lipophilicity and polar surface area. It was further striking to observe that 17 of 19 carboxylic acids tested evaded efflux by P-gp. While the use of carboxylic acid in the context of enhancing CNS penetrance is counterintuitive, there are several approved CNS drugs with carboxylic acids, and some CNS preclinical studies of acid-containing compounds achieving appreciable penetration to the brain.

To test the hypothesis, a series of secondary experiments were performed. Evaluating acid-containing compounds' transport in the isogenic cell line without P-gp overexpression demonstrated no other modes of transport, and calcein-AM assay with the fluorescent P-gp substrate showed that compounds generally do not inhibit the efflux activity. Co-administration of P-gp inhibitor illustrated that while cetirizine, an acid-containing P-gp substrate and an outlier, reduces its efflux, efflux ratios of other acids do not change and thus validate their non-substrate classification. A total of 101 carboxylic acid containing compounds have been evaluated and over 85% of compounds with appreciable permeability are not recognized by P-gp. Most acids showed various levels of permeability while maintaining lack of P-gp recognition, illustrating that the evasion of efflux is not due to low permeability of these compounds.

Conversion of P-gp substrates into non-substrates. To investigate the actionability of the observation that most acids evade efflux, several series of compounds recognized by P-gp were considered for proof-of-concept studies. Upon appending carboxylic acid moieties to these substrates, P-gp recognition decreases, albeit at lower permeability, across five examples from different structural classes. Additional examples of conversions, as well as parent substrates bearing simple methyl groups for another comparison are under investigation. In efforts to increase overall permeability of acids, multiple series of compounds with different spatial orientation of the charge and isosteres are investigated.

Actionability in animals. Given the complexity of the BBB in vivo, the observations from in vitro experiments needed validation in an animal model. To this end, an in vivo P-gp assay was adapted, optimized and validated. Briefly, mice were pretreated with a potent P-gp inhibitor, elacridar, or its vehicle, then a compound of interest was administered via lateral tail vein. At each time point, blood and brain were harvested, and compound concentrations in each compartment were quantified.

In this experiment, brain-to-blood ratios of P-gp substrates decrease with the P-gp inhibitor compared to vehicle, while those of non-substrates will remain the same.

With this assay in hand, an acid-containing indole alkaloid that exhibited high permeability and low efflux in vitro was initially evaluated. P-gp inhibition did not affect its disposition in the brain at 5, 15 and 60 minutes, thus validating observations from the transwell assays. Second, another acid-containing compound, levofloxacin was evaluated. From in vitro experiments, levofloxacin has been shown to evade P-gp efflux (ER=0.62) with appreciable permeability. As a comparison, methyl-ester derivative of levofloxacin, which was recognized by P-gp with efflux ratio of 5.9, was considered.

The P-gp inhibitor co-treatment had a negligible effect on the brain exposure of levofloxacin at both timepoints, but significantly potentiated the methyl-ester counterpart of levofloxacin, validating the in vitro data that levofloxacin evades P-gp efflux but levofloxacin-ME is actively pumped out of the brain by P-gp. Comparing the brain exposure of the two compounds in the absence of P-gp inhibitor, levofloxacin had statistically superior partition to the brain compared to P-gp recognized levofloxacin-ME after 60 min, although they had comparable partitioning at 5 min. Taken together, this experiment demonstrated that a compound with carboxylic acid moiety evades P-gp efflux in vivo.

Conversion of non-BBB penetrant drugs. Finally, the findings from experimental observations were applied to existing anti-proliferative kinase inhibitors with limited CNS exposure due to P-gp efflux, such as vemurafenib and imatinib, to yield much needed improved compounds for treating metastatic lesions in the brain.

Toward this end, dabrafenib emerged as an attractive candidate for the proof of concept. Dabrafenib is a potent, selective and efficacious inhibitor targeting ^{V600E}BRAF and is approved for treatment of BRAF mutant melanomas. Recognized by P-gp at efflux ratio of 11.4-18.3, the brain accumulation of dabrafenib is limited. With its enhanced disposition in animals lacking P-gp and enhanced efficacy in mice treated with a blood-brain barrier permeabilizer, a derivative that evades P-gp efflux while maintaining permeability, a brain-penetrant ^{V600E}BRAF inhibitor could be achieved. From structure-activity relationship studies in the literature, supplemented by the docking studies with the crystal structure, modifications at the amino group on the pyrimidine ring appeared to be amenable. Derivatives with varying linker at the tail position were synthesized (see Scheme 2 and Scheme 3 to 8 in Example 2). All acid-containing derivatives demonstrated reduced P-gp efflux and revealed the distance for the acid moiety from the core to retain the target engagement.

Inspired by other BRAF inhibitors, vemurafenib and encorafenib, new compounds were synthesized by manipulating their size and lipophilicity, and ultimately led to compounds with nanomolar potency. In a panel of cell-lines harboring ^{WT}BRAF and ^{V600E}BRAF, these demonstrated a comparable selectivity to that of approved BRAF inhibitors, while reducing their efflux ratios.

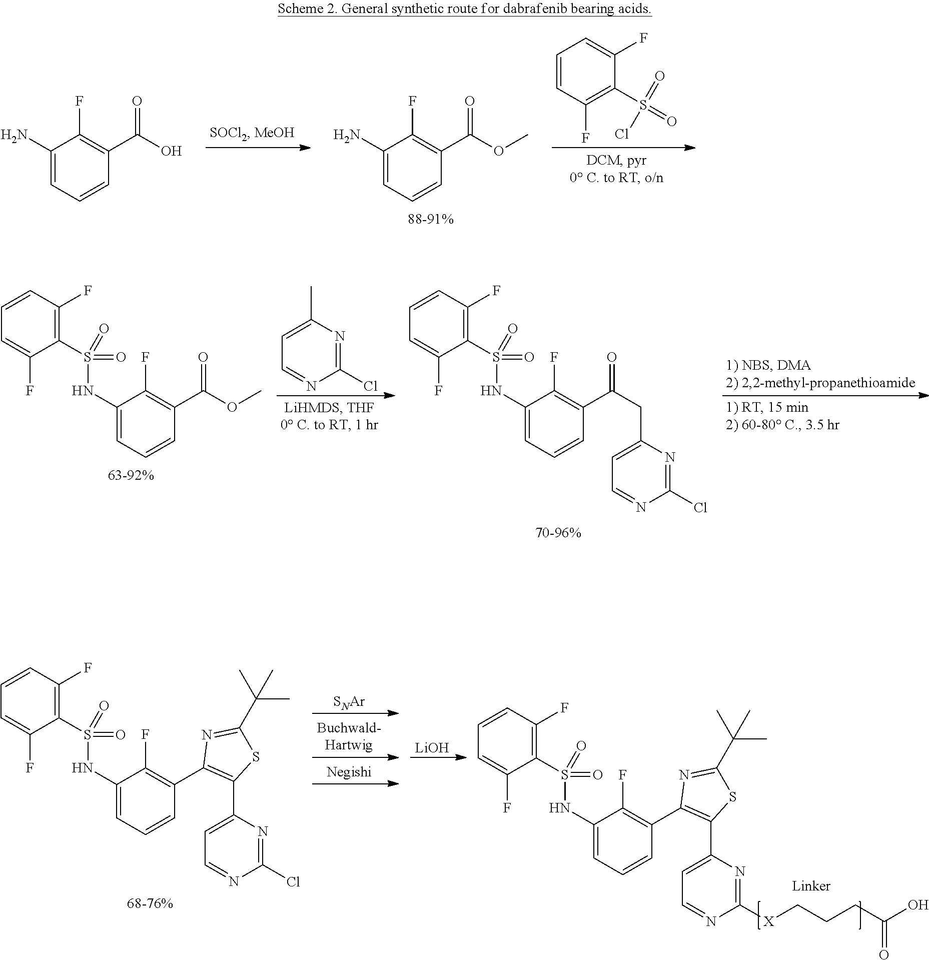

For the synthesis shown in Scheme 2, see Bioorg. Med. Chem. Lett. 2011, 21, 4436. Following the esterification of 3-amino-benzoic acid, anilines were reacted with sulfonyl chloride to form the sulfonamide. The ester was then condensed with 2-chloro-methyl pyrimidine to generate the ketone intermediate. Bromination of the ketone with NBS, followed by Hantzsch reaction installed the thiazole core. This chloropyrimidine intermediate was then subjected to a variety of substitution conditions (including S_{N}Ar, and Buckwald-Hartwig, Negishi, and Suzuki couplings) followed by saponification to yield the final acids.

Additional results from the evaluation of compounds disclosed herein are shown in Table 5 to Table 13.

TABLE 5
Evaluation of first-generation dabrafenib derivatives bearing carboxylic acid with various linkers (6-74 = Dabrafenib).

| Compound | 6-74 | 6-77 | 6-85 | 6-89 | 6-91 | 6-97 | 6-86 | 6-83 |
|---|---|---|---|---|---|---|---|---|
| A375 IC₅₀ (nM) | 5.380 ± 0.38 | >1000 | 474.4 ± 11.8 | 166.36 ±4.09 | >1000 * | >1000 | 628.4 ± 105 | 114.6 ± 9.85 |
| P_{app A-B} (nm/s) | 12.57 ± 6.72 | 15.82 ± 3.35 | 7.09 ± 0.45 | 22.37 ± 1.26 | 4.93 ± 1.48 | 8.68 ± 0.21 | 8.44 ± 2.14 | 10.89 ± 0.97 |
| P_{app B-A} (nm/s) | 131.92 ± 16.32 | 14.47 ± 0.77 | 22.59 ± 3.20 | 12.51 ± 4.36 | 6.26 ± 2.38 | 7.41 ± 1.56 | 4.45 ± 0.40 | 21.65 ± 3.71 |
| ER | 17.94 ± 6.97 | 0.984 ± 0.164 | 3.22 ± 0.54 | 0.56 ± 0.18 | 1.30 ± 0.38 | 0.86 ± 0.19 | 0.607 ± 0.16 | 1.96 ± 0.22 |

72-hour incubation. A375 3000 c/w, Cell viability assessed by Alamar Blue. n = 3. MDR1-MDCKII cells seeded 300,000 cells/cm² for 80-96 hour Treated with 10 μM compound for 90 min at 37° C. w/90 rpm Membrane integrity checked by LY n = 3. Error indicates SEM. Quinidine ER = 8.09 ± 0.56, Propranolol ER = 1.18 ± 0.03.

TABLE 6
Evaluation of first-generation dabrafenib derivatives bearing carboxylic acid with various linkers (Cont'd).

| Compound | Dabrafenib | 6-83 | 6-121 | 6-191 | 6-86 | 6-131 | 6-193 |
|---|---|---|---|---|---|---|---|
| A375 IC₅₀ (nM) | 5.380 ± 0.38 | 114.6 ± 9.85 | 1268 ± 54.4 | 557.8 ± 118 | 628.4 ± 105 | 1583 ± 458 | 1749 ± 21.9 |
| % Eₘₐₓ | 65 | 65 | 60 | 60 | 65 | 60 | 60 |
| P_{app A-B} (nm/s) | 12.57 ± 6.72 | 10.89 ± 0.97 | N/D | N/D | 8.44 ± 2.14 | 4.49 ± 0.08 | N/T |
| P_{app B-A} (nm/s) | 131.92 ± 16.32 | 21.65 ± 3.71 | 3.25 ±1.24 | 14.53 ± 1.72 | 4.45 ± 0.40 | 10.34 ± 1.66 | N/T |
| ER | 17.94 ± 6.97 | 1.96 ± 0.22 | — | — | 0.607 ± 0.16 | 1.933 ± 0.08 | N/T |

N/T: Not tested, N/D: Not determined. 72-hour incubation. A375 3000 c/w, Cell viability assessed by Alamar Blue. n = 3. MDR1-MDCKII cells seeded 300,000 cells/cm² for 80-96 hour Treated with 10 μM compound for 90 min at 37° C. w/90 rpm Membrane integrity checked by LY n = 3. Error indicates SEM. Quinidine ER = 8.09 ± 0.56, Propranolol ER = 1.18 ± 0.03.

TABLE 7
Evaluation of second-generation dabrafenib derivatives with
the core structure modifications bearing carboxylic acid.

| Compound | 6-166 | 6-181 | 6-167 | 6-179 | 6-163 | 6-173 | 6-145 | 6-150 |
|---|---|---|---|---|---|---|---|---|
| A375 IC₅₀ (nM) | >3000 | >50000 | >3000 | 14710 ± 6164 | >3000 | >50000 | >3000 | >50000 |
| P_{app A-B} (nm/s) | N/T | N/T | N/T | N/D | N/T | N/D | 9.4 ± 0.5 | 5.18 ± 0.0 |
| P_{app B-A} (nm/s) | N/T | N/T | N/T | 17.86 ± 0.64 | N/T | 4.60 ± 0.86 | 56.6 ± 1.9 | 3.98 ± 1.49 |
| ER | N/T | N/T | N/T | — | N/T | — | 6.05 ± 0.50 | 0.6 |

N/T: Not tested, pKa calculated using ChemAxon software N/D: Not determined. 72-hour incubation. 50 mM Raptinal used as dead control. A375 3000 c/w, Cell viability assessed by Alamar Blue. n = 3. Error indicates SEM.

TABLE 8
Evaluation of second-generation dabrafenib derivatives with the
core structure modifications bearing carboxylic acid (Cont’d).

| Compound | 6-83 | 6-244 | 6-249 | 6-251 |
|---|---|---|---|---|
| A375 IC₅₀ (nM) | 114.6 ± 9.85 | 4.113 ± 0.482 | 531.33 ± 32.1 | 1007 ± 97.3 |
| P_{app A-B} (nm/s) | 10.89 ± 0.97 | N/T | N/T | N/T |
| P_{app B-A} (nm/s) | 21.65 ± 3.71 | N/T | N/T | N/T |
| ER | 1.96 ± 0.22 | N/T | N/T | N/T |

72 hr incubation. A375 3000 c/w, Cell viability assessed by Alamar Blue. n = 3. Error indicates SEM.

TABLE 9
Evaluation of hybrid compounds (third generation).

| Compound | Vemurafenib | Dabrafenib | Encorafenib | 6-261 | 6-263 |
|---|---|---|---|---|---|
| P_{app A-B} (nm/s) | 6.67 ± 1.10 | 12.6 ± 6.72 | 13.2 ± 6.02 | 49.2 ± 8.06 | 21.5 ± 0.70 |
| P_{app B-A} (nm/s) | 38.1 ± 0.63 | 132 ± 16.3 | 147 ± 18.0 | 64.2 ± 24.5 | 24.9 ± 4.35 |
| ER | 5.92 ± 0.77 | 17.9 ± 6.97 | 21.8 ± .10.4 | 1.40 ± 0.65 | 1.17 ± 0.22 |
| A375 IC₅₀ (nM) | 331 ± 93.3 | 7.46 ± 1.10 | 5.17 ± 0.412 | 2.80 ± 0.532 | 27.3 ± 3.01 |

72-hour IC₅₀ values (nM) of the tail-group modified dabrafenib derivatives in the A375 BRAF V600E mutant melanoma cell line. Cell viability was assessed via the Alamar Blue assay. Error is the standard error of the mean (SEM), n = 3. Permeability (Papp) and efflux ratio were measured in the MDR1-MDCKII transwell assay at 10 μM. Error is SEM, n = 3. P-gp substrates (ER ≥ 2). Non-substrates (ER < 2). Low-permeable compounds (Papp B-A < 15 nm/s).

TABLE 10
Evaluation of hybrid compounds (Cont’d).

| Compound/(IC₅₀) | Dabrafenib | 6-265 | 6-263 | 8-41 | 8-43 |
|---|---|---|---|---|---|
| A375 (V600E) (nM) | 7.46 ± 1.10 | 113 ± 10.7 | 27.3 ± 3.01 | 301 ± 39.9 | 38.2 ± 3.29 |
| SK-MEL-28 (V600E) (nM) | 4.76 ± 0.416 | 124 ± 4.86 | 22.5 ± 1.20 | 246 ± 8.729 | 51.1 ± 1.35 |
| HCT116 (WT) (mM) | >1 mM | 8.84 ± 0.00462 | 2.10 ± 0.456 | >1 mM | 5.13 ± 0.382 |

72-hour IC₅₀ values (nM) of the tail-group modified dabrafenib derivatives in the A375 BRAF V600E mutant melanoma cell line. Cell viability was assessed via the Alamar Blue assay. Error is the standard error of the mean (SEM), n = 3. Permeability (Papp) and efflux ratio were measured in the MDR1-MDCKII transwell assay at 10 μM. Error is SEM, n = 3. P-gp substrates (ER ≥ 2). Non-substrates (ER < 2). Low-permeable compounds (Papp B-A < 15 nm/s).

TABLE 11
IC₅₀ of 261 vs. dabrafenib in various cells.

| IC₅₀ | Tissue Origin | Dabrafenib | 6-261 |
|---|---|---|---|
| A375 (V600E) (nM) | Melanoma | 7.46 ± 1.10 | 2.80 ± 0.532 |
| SK-MEL-28 (V600E) (nM) | Melanoma | 4.76 ± 0.416 | 2.35 ± 0.128 |
| HT29 (V600E) (nM) | Colon | 4.74 ± 0.844 | 1.67 ± 0.363 |
| AM-38 (V600E) (nM) | Glioma | 6.84 ± 0.836 | 8.50 ± 1.17 |
| CHL-1 (WT) (mM) | Melanoma | >1 mM | >1 mM |
| B16F10 (WT) (mM) | Melanoma | >1 mM | >1 mM |
| HCT116 (WT) (mM) | Colon | >1 mM | >1 mM |
| T98G (WT) (mM) | Glioma | >1 mM | >1 mM |
| U118MG (WT) (mM) | Glioma | >1 mM | >1 mM |
| U87 (WT) (mM) | Glioma | >1 mM | >1 mM |

72-hour IC₅₀ curves of 261 compared to dabrafenib in a panel of ten cell lines. Cell viability was assessed via the Alamar Blue assay. Error is the standard error of the mean (SEM), n = 3.

TABLE 12
IC₅₀ of 261 vs. vemurafenib in various cells.

| IC₅₀ | Vemurafenib | 6-263 |
|---|---|---|
| A375 (V600E) (nM) | 331 ± 93.3 | 27.3 ± 3.01 |
| SK-MEL-28 (V600E) (nM) | 46.6% dead at 10 mM | 22.5 ± 1.20 |
| HT29 (V600E) (nM) | n.d. | 21.4 ± 4.21 |
| AM-38 (V600E) (nM) | 344 ± 59.3 | 93.2 ± 14.1 |
| CHL-1 (WT) (mM) | >10 mM | >10 mM |
| B16F10 (WT) (mM) | n.d. | >10 mM |
| HCT116 (WT) (mM) | 27 ** | 2.10 ± 0.456 |
| T98G (WT) (mM) | 49.5% dead at 10 mM | >10 mM |
| U118MG (WT) (mM) | >10 mM | >10 mM |
| U87 (WT) (mM) | >10 mM | >10 mM |

72-hour IC50 curves of 263 compared to vemurafenib in a panel of ten cell lines. Cell viability was assessed via the Alamar Blue assay. Error is the standard error of the mean (SEM), n = 3.

TABLE 13
Pharmacokinetics analysis of 261.

| | |
|---|---|
| Time | [Drug] (nM) |

| (min) | 20 mg/kg IP | 25 mg/kg IV |
|---|---|---|
| 0 | 0 | |
| 15 | 12900 ± 557 | 16375 ± 719 |
| 30 | 9463 ± 621 | |
| 45 | 6703 ± 122 | |
| 60 | 5590 ± 167 | 6557 ± 290 |
| 120 | 2550 ± 194 | |
| 240 | 581 ± 118 | |
| 480 | 14 ± 2.07 | |
| 960 | 0 | |

| Parameter | Units | Estimate |
|---|---|---|
| AUC | min*ng/ml | 503964.63 |
| T_{1/2} | min | 37.301672 |
| Cmax | ng/ml | 9364.7721 |
| CL | ml/min/kg | 39.685325 |
| Vss | ml/kg | 2692.9321 |

Strain: Female CD-1 (n ≥ 3 per time point). Dose: 20 mg/kg. Formulation: 10% DMSO, 40% PEG400, 50% 5% HPβCD. Route: Intraperitoneal.

### Example 2. Materials and Methods for Chemical Synthesis

Chemical reagents were purchased from commercial sources and used without further purification. Anhydrous solvents were either purchased from commercial suppliers or dried after being passed through columns packed with activated alumina under positive pressure of nitrogen using a PureSolv MD-5 (Inert, previously Innovative Technology Inc.) solvent purification system. The reverse-phase purification was performed on a Biotage Isolera using Agela Technologies AQ C18 spherical 20-35 m 100A columns (12 g cartridge with 12 mL/min flow rate) with gradient elution of H2O:MeCN with or without 0.1% formic acid. Microwave reactions were performed using Anton Paar Monowave 400 Microwave Synthesis Reactor. ¹H NMR, ¹³C NMR, and ¹⁹F NMR experiments for prepared intermediates and products were recorded on a Bruker Advance III HD 500 MHz NMR system equipped with a CryoProbe or a Bruker NEO 600 MHz NMR system equipped with a Prodigy probe. Spectra were obtained in the following solvents with reference peaks included for ¹H and ¹³C NMRs: CDCl3 (¹H NMR 7.26 ppm; ¹³C NMR 77.16 ppm), DMSO-d⁶ (¹H NMR 2.50 ppm; ¹³C NMR 39.52 ppm), CD₃OD (¹H NMR: 3.31 ppm; ¹³C NMR: 49.00 ppm).Chemical shift values are expressed in ppm (δ), coupling constants (J, Hz) and peak patterns are reported as broad singlet (bs), singlet (s), doublet (d), triplet (t), quartet (q), pentet (p), heptet (hept), and multiplet (m). High resolution mass spectra (HRMS) were obtained in the School of Chemical Sciences Mass Spectrometry Laboratory on a Waters Q-TOF Ultima quadrupole time of flight spectrometer using electrospray ionization ESI.

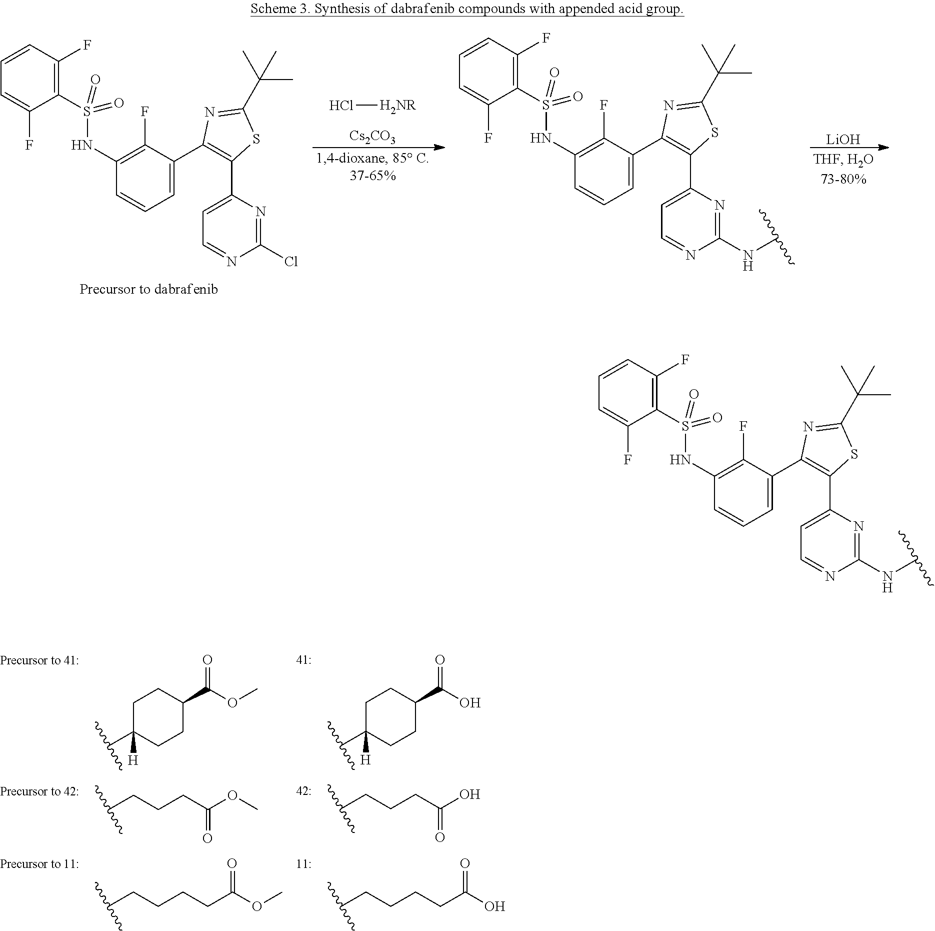

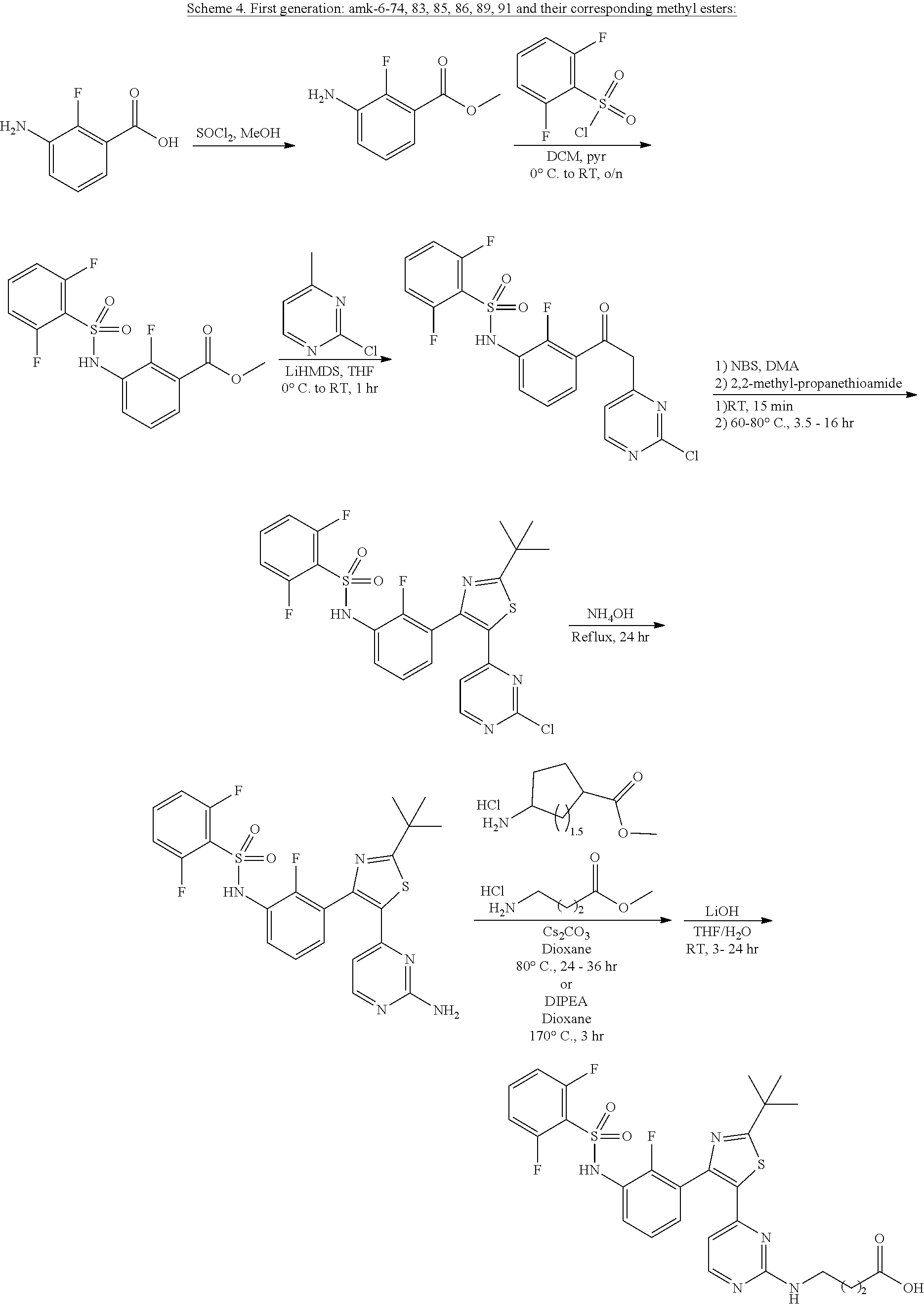

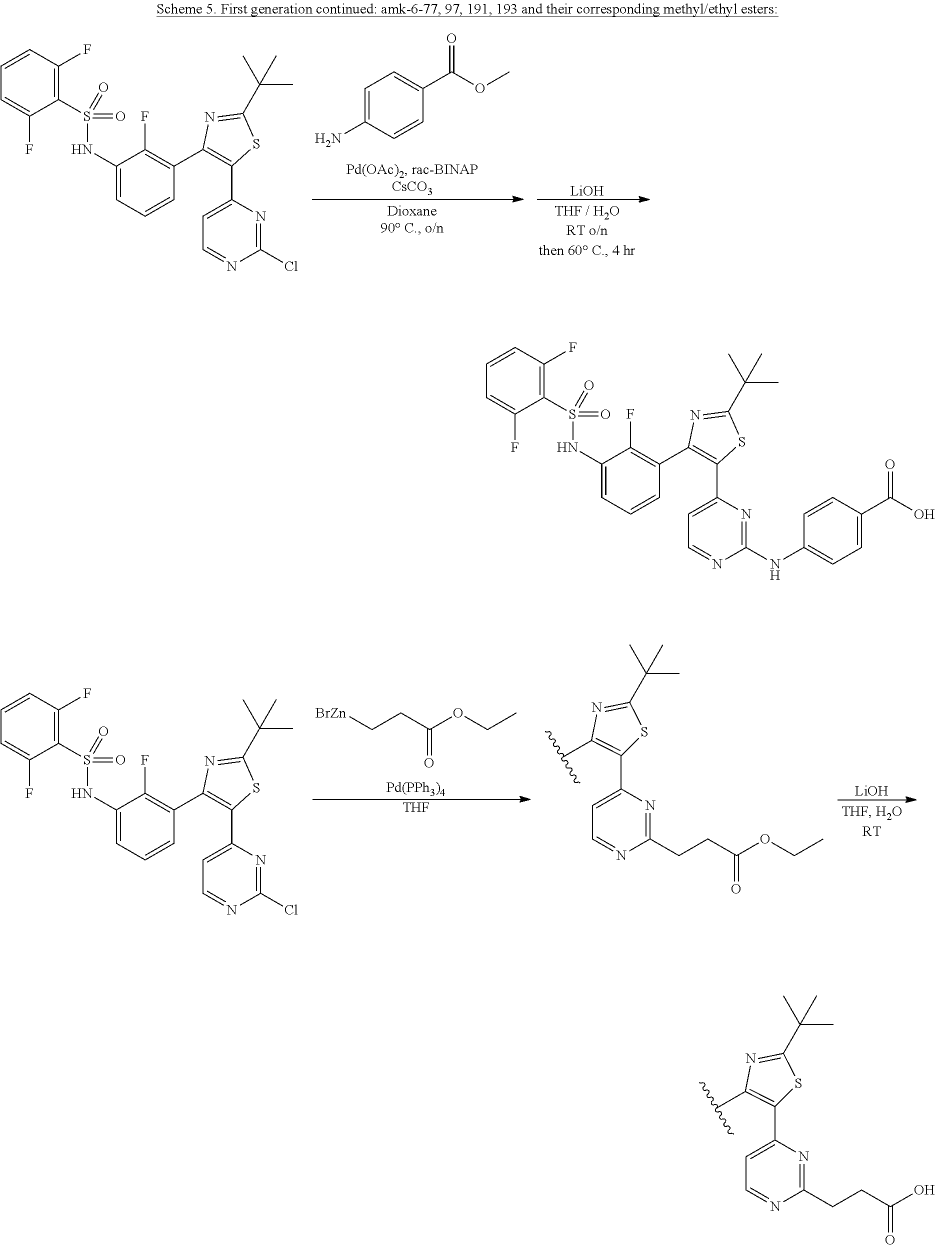

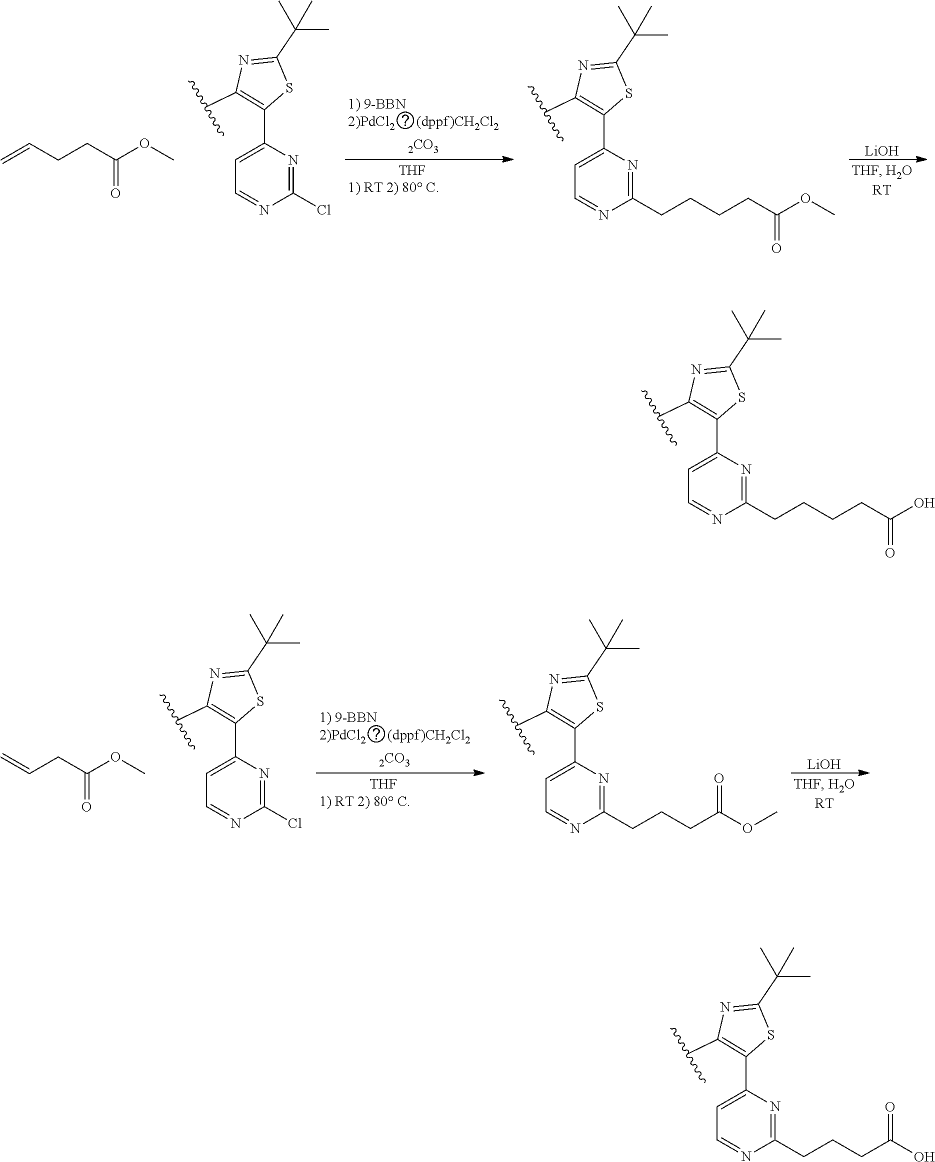

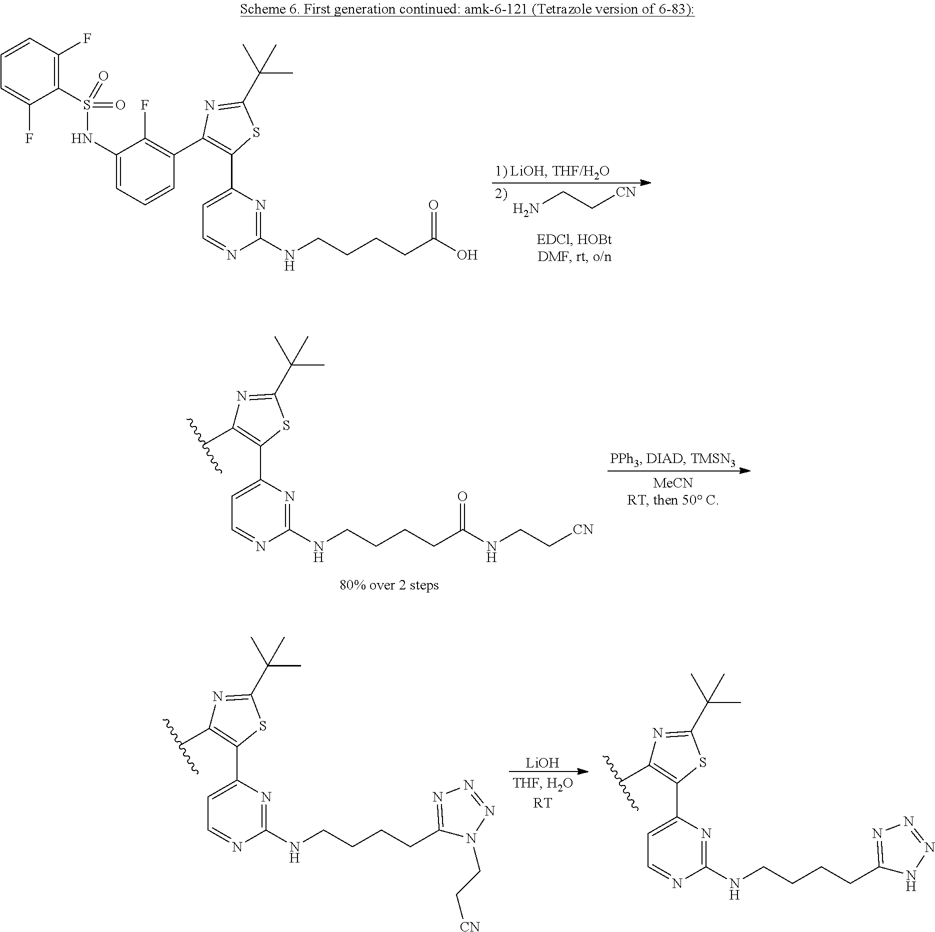 [SMILES: CC(C)(C)c1nc(-c2cccc(NS(=O)(=O)c3c(F)cccc3F)c2F)c(-c2ccnc(NCCCCC(=O)O)n2)s1 ; N#CCCN ; *c1nc(C(C)(C)C)sc1-c1ccnc(NCCCCC(=O)NCCC#N)n1 ; *c1nc(C(C)(C)C)sc1-c1ccnc(NCCCCc2nnnn2CCC#N)n1 ; "LiOH" ; *c1nc(C(C)(C)C)sc1-c1ccnc(NCCCCc2nnn[nH]2)n1]

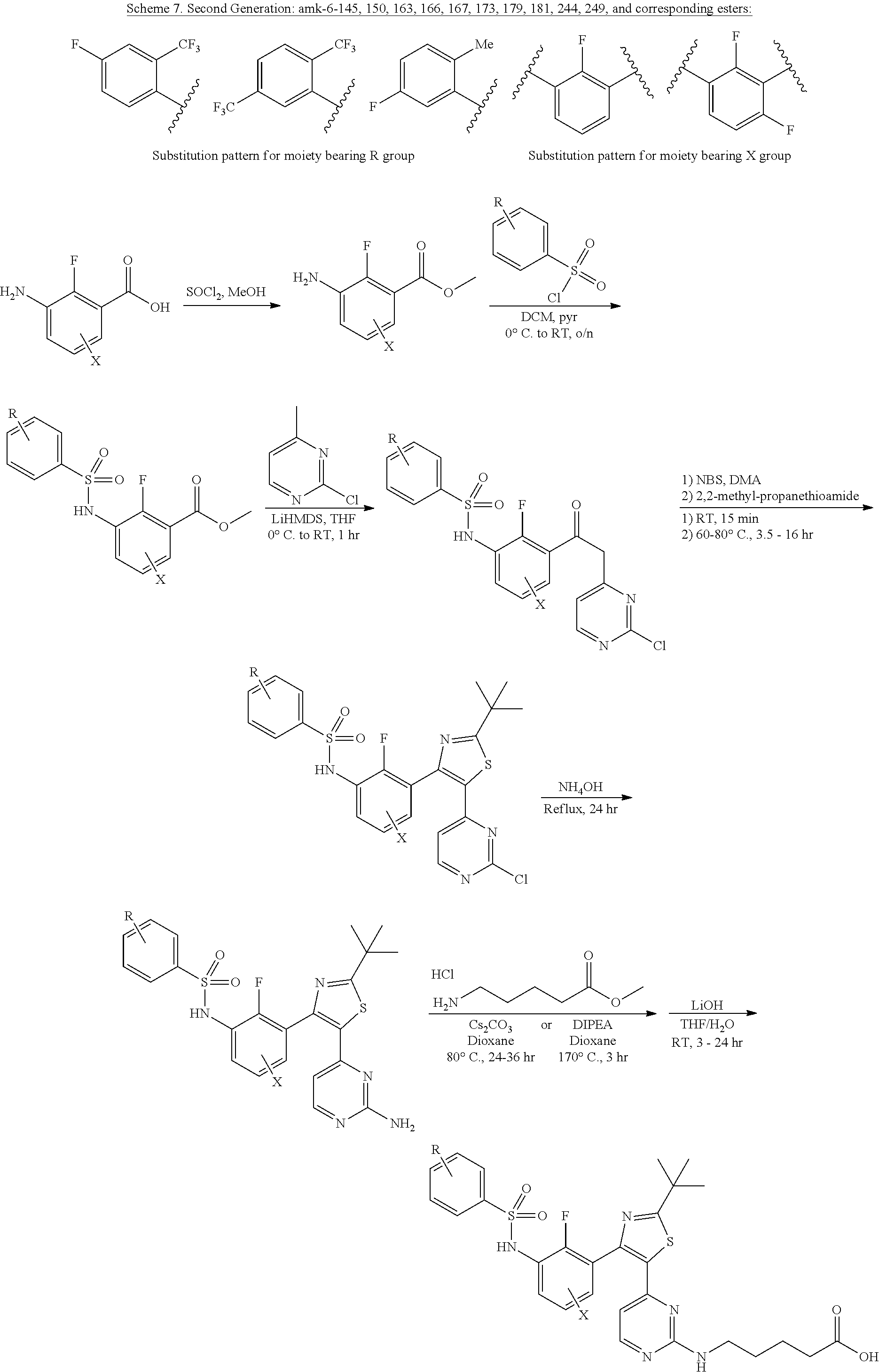

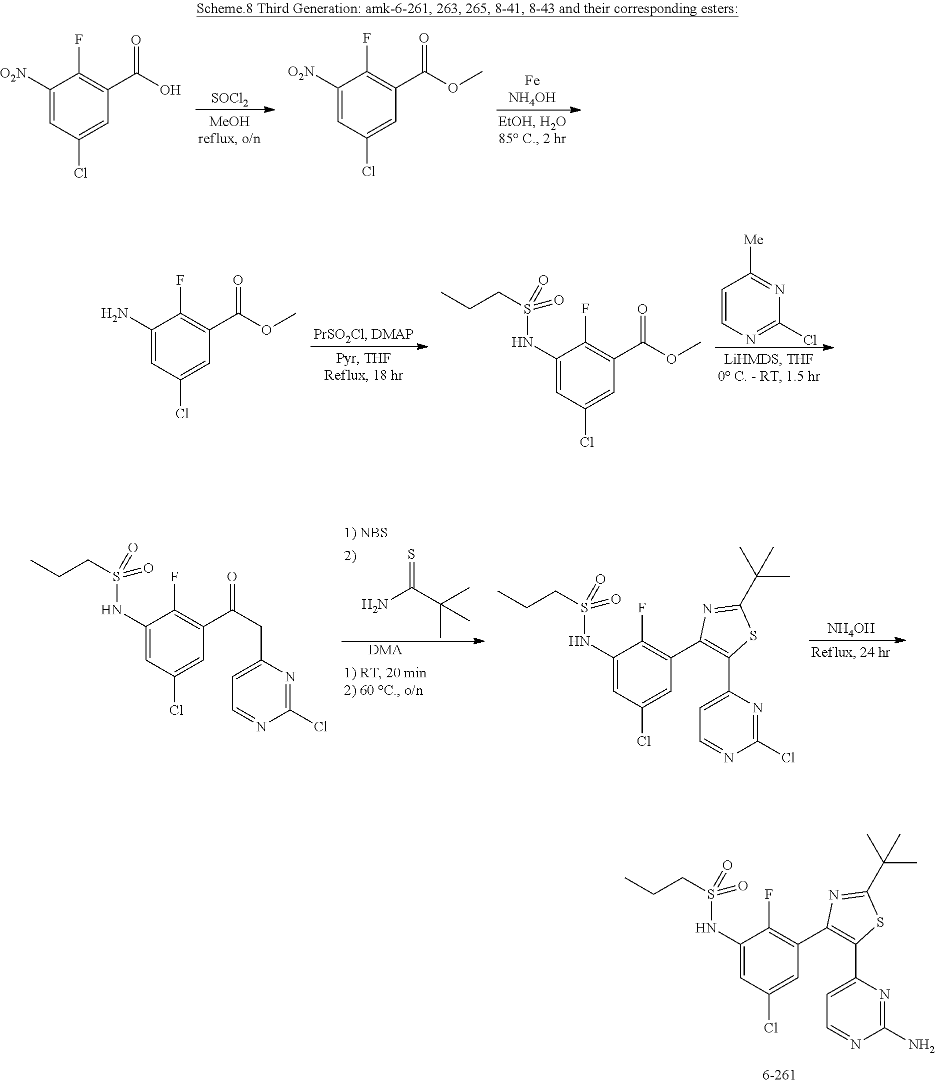

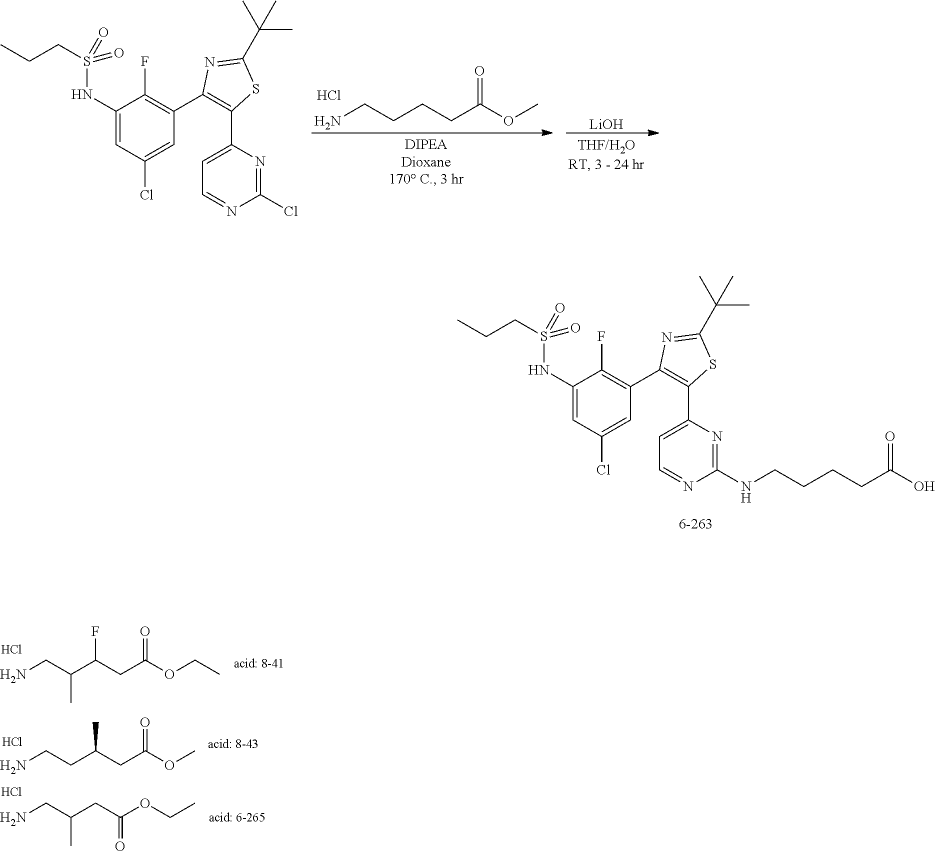

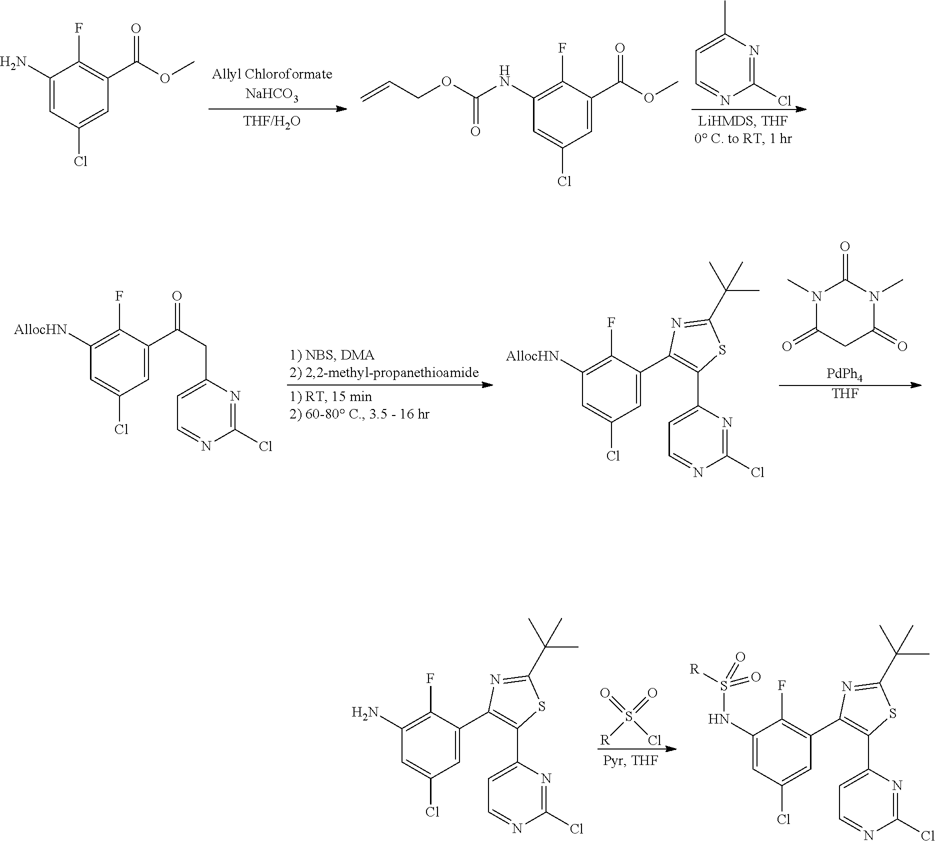

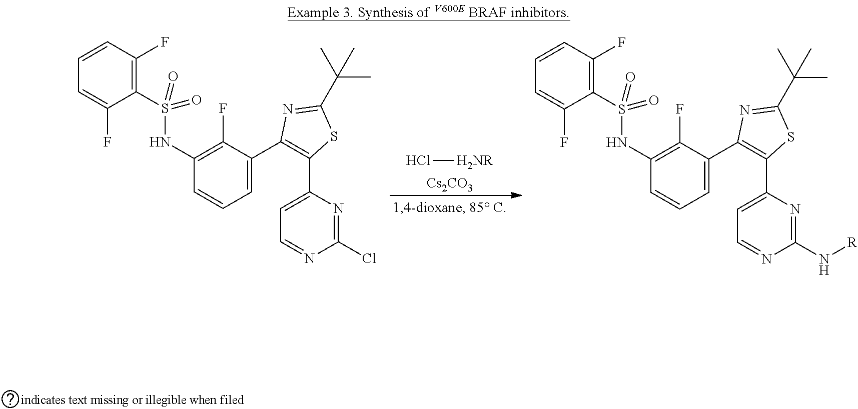

General Procedure. In an oven-dried vial with a stir bar, chloropyrimidine intermediate (1 eq.), amine hydrochloride (4 eq.) and cesium carbonate (7 eq.) were dissolved in anhydrous 1,4-dioxane. The vial was sealed and heated at 85° C. until completion. The reaction was then diluted in ethyl acetate and 1 μM hydrochloric acid. The organic layer was extracted with ethyl acetate thrice, and the combined organics were washed with brine and dried over anhydrous magnesium sulfate, filtered, and concentrated in vacuo. The resulting residue was purified via silica gel chromatography to yield the desired product.

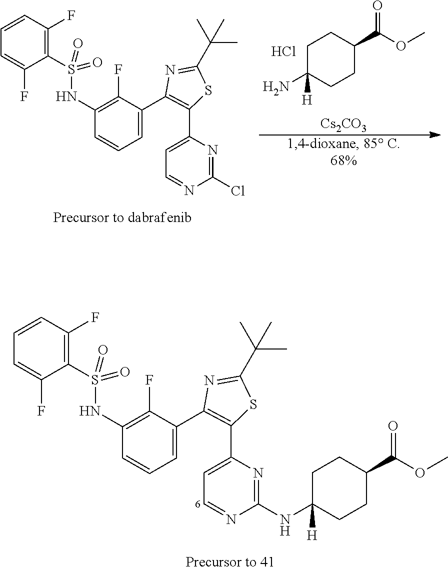

Methyl (1r,4r)-4-((4-(2-(tert-butyl)-4-(3-((2,6-difluorophenyl)sulfonamido)-2-fluorophenyl)thiazol-5-yl)pyrimidin-2-yl)amino)cyclohexane-1-carboxylate (precursor to 41). Synthesized from the precursor to dabrafenib and methyl trans-4-aminocyclohexane-carboxylate hydrochloride according to the general procedure to yield the precursor to 41 (124 mg, 68%) as a yellow solid. ¹H NMR (500 MHz, CDCl3) δ 7.95-7.89 (m, 1H), 7.74-7.68 (m, 1H), 7.47 (dqd, J=8.4, 6.1, 2.9 Hz, 1H), 7.33 (d, J=7.1 Hz, 1H), 7.21 (td, J=8.0, 2.2 Hz, 1H), 6.96 (tt, J=9.2, 1.6 Hz, 2H), 6.06 (s, 1H), 5.20 (s, 1H), 3.69 (s, 3H), 2.29 (ddt, J=15.3, 12.0, 3.4 Hz, 1H), 2.18-2.09 (m, 2H), 2.06-1.99 (m, 2H), 1.57 (q, J=13.0 Hz, 2H), 1.46 (s, 9H), 1.20 (q, J=12.6 Hz, 2H). ¹³C NMR (126 MHz, CDCl3) δ 182.93, 176.22, 161.31, 160.94 (d, J=3.5 Hz), 158.95, 158.87 (d, J=3.4 Hz), 158.06, 151.08 (d, J=247.8 Hz), 145.75, 135.33 (t, J=11.0 Hz), 134.34, 128.28, 125.30 (d, J=4.6 Hz), 124.74, 124.63, 123.20, 117.07, 113.40 (d, J=3.7 Hz), 113.22 (d, J=3.7 Hz), 106.36, 51.81, 49.46, 42.57, 38.18, 32.16, 30.87, 27.95 (27 carbons due to symmetry). ¹⁹F NMR (471 MHz, CDCl3) δ-106.84 (dt, J=9.9, 4.9 Hz), -130.46. HRMS(ESI): m/z calc. for C31H32F3N504S2 [M+H]⁺: 660.1921, found: 660.1914.

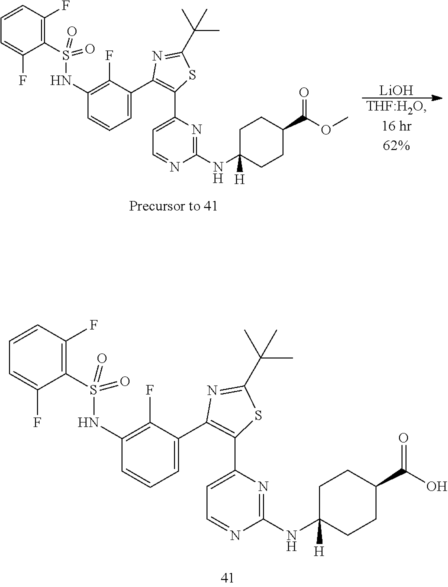 [SMILES: COC(=O)[C@H]1CC[C@H](Nc2nccc(-c3sc(C(C)(C)C)nc3-c3cccc(NS(=O)(=O)c4c(F)cccc4F)c3F)n2)CC1 ; "LiOH" ; "THF:H2O,16 hr62%" ; CC(C)(C)c1nc(-c2cccc(NS(=O)(=O)c3c(F)cccc3F)c2F)c(-c2ccnc(N[C@H]3CC[C@H](C(=O)O)CC3)n2)s1]

(1r,4r)-4-((4-(2-(tert-butyl)-4-(3-((2,6-difluorophenyl)sulfonamido)-2-fluorophenyl)thiazol-5-yl)pyrimidin-2-yl)amino)cyclohexane-1-carboxylic acid (41). Prepared from the precursor to 41 according to a general hydrolysis procedure (trituration) to yield 41 (27 mg, 62%) as an off-white solid. ¹H NMR (500 MHz, DMSO) δ 10.87 (s, 1H), 8.03 (d, J=5.2 Hz, 1H), 7.67 (tt, J=8.4, 5.9 Hz, 1H), 7.42 (s, 1H), 7.36 (t, J=6.7 Hz, 1H), 7.25 (dt, J=21.5, 8.6 Hz, 3H), 7.15 (d, J=7.7 Hz, 1H), 5.87 (d, J=91.0 Hz, 1H), 2.13 (t, J=11.9 Hz, 1H), 1.91 (s, 3H), 1.40 (s, 9H), 1.38-1.16 (m, 5H) (28 non-exchangeable protons). ¹³C NMR (126 MHz, MeOD and DMSO) δ 184.00, 179.10, 162.65, 161.88 (d, J=3.8 Hz), 159.83 (d, J=3.7 Hz), 159.69, 159.41, 154.10 (d, J=249.7 Hz), 147.23, 136.77 (t, J=11.1 Hz), 135.68, 129.92, 127.44, 126.08 (d, J=13.4 Hz), 125.88 (d, J=4.4 Hz), 118.92 (t, J=15.9 Hz), 114.36 (d, J=3.6 Hz), 114.17 (d, J=3.7 Hz), 106.82, 50.57, 43.63, 39.03, 32.71, 31.06 (2 overlapping peaks), 29.26 (26 carbons due to symmetry). ¹⁹F NMR (471 MHz, MeOD) δ-108.48 (dt, J=9.6, 4.4 Hz), -127.80. HRMS(ESI): m/z calc. for C30H31F3N504S2 [M+H]⁺: 646.1764, found: 646.1762.

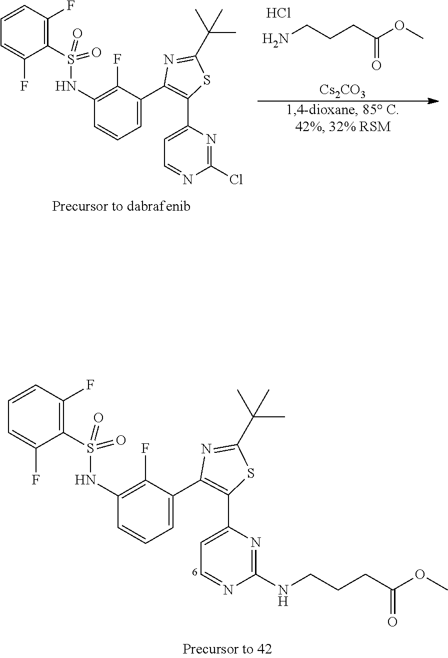

Methyl 4-((4-(2-(tert-butyl)-4-(3-((2,6-difluorophenyl)sulfonamido)-2-fluorophenyl)thiazol-5-yl)pyrimidin-2-yl)amino)butanoate (precursor to 42). Synthesized from precursor to dabrafenib and methyl 4-aminobutanoate hydrochloride according to the general procedure to yield the precursor to 42 (48 mg, 42%) as a pale-yellow solid and recovered starting material (32%). ¹H NMR (500 MHz, CDCl3) δ 7.96 (d, J=5.3 Hz, 1H), 7.71 (ddd, J=8.6, 7.5, 1.7 Hz, 1H), 7.56-7.38 (m, 1H), 7.31 (ddd, J=8.0, 6.5, 1.7 Hz, 1H), 7.20 (td, J=7.9, 1.0 Hz, 1H), 7.00-6.93 (m, 2H), 6.07 (d, J=5.3 Hz, 1H), 5.29 (bs, 1H), 3.68 (s, 3H), 3.38 (s, 2H), 2.41 (t, J=7.3 Hz, 2H), 1.92 (p, J=7.1 Hz, 2H), 1.46 (s, 9H) (27 non-exchangeable protons). ¹³C NMR (126 MHz, CDCl3) δ 182.89, 173.95, 162.00, 160.75 (d, J=3.3 Hz), 159.02 (d, J=3.3 Hz), 158.83, 158.07, 151.21 (d, J=248.1 Hz), 145.81, 135.32 (t, J=11.1 Hz), 134.27, 128.35, 125.23 (d, J=4.3 Hz), 124.67, 124.58, 123.40, 117.08 (t, J=15.3 Hz), 113.37 (d, J=3.6 Hz), 113.22 (d, J=3.6 Hz), 106.52, 51.82, 40.78, 38.15, 31.57, 30.85, 24.89 (26 carbons due to symmetry). ¹⁹F NMR (471 MHz, CDCl3) δ-106.85 (dt, J=9.8, 4.9 Hz), -130.25 (td, J=6.6, 3.1 Hz). HRMS(ESI): m/z calc. for C28H29F3N504S2 [M+H]⁺: 620.1608, found: 620.1605.

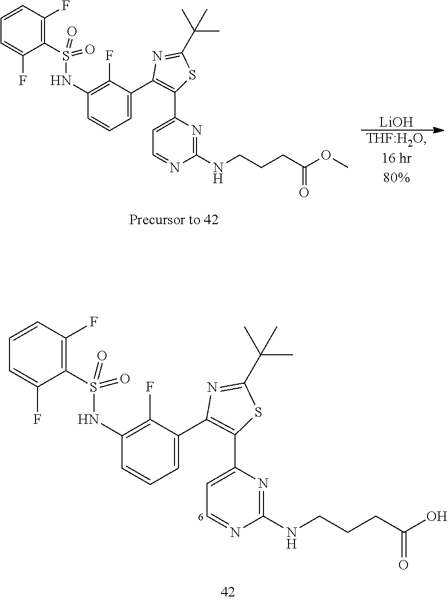 [SMILES: COC(=O)CCCNc1nccc(-c2sc(C(C)(C)C)nc2-c2cccc(NS(=O)(=O)c3c(F)cccc3F)c2F)n1 ; "LiOH" ; "THF:H2O,16 hr80%" ; CC(C)(C)c1nc(-c2cccc(NS(=O)(=O)c3c(F)cccc3F)c2F)c(-c2ccnc(NCCCC(=O)O)n2)s1]

4-((4-(2-(tert-butyl)-4-(3-((2,6-difluorophenyl)sulfonamido)-2-fluorophenyl)thiazol-5-yl)pyrimidin-2-yl)amino)butanoic acid (42). Prepared from the precursor to 42 according to a general hydrolysis procedure (trituration) to yield 42 (18 mg, 80%) as an off-white solid. ¹H NMR (500 MHz, MeOD) δ 7.99 (s, 1H), 7.60 (ddt, J=11.7, 9.5, 3.9 Hz, 2H), 7.36 (ddd, J=7.9, 6.2, 1.7 Hz, 1H), 7.29 (t, J=7.9 Hz, 1H), 7.09 (t, J=9.1 Hz, 2H), 6.19 (s, 1H), 3.39-3.34 (m, 2H), 2.39 (t, J=7.3 Hz, 2H), 1.89 (p, J=7.0 Hz, 2H), 1.49 (s, 9H) (23 non-exchangeable protons). ¹H NMR (500 MHz, DMSO) δ 10.87 (s, 1H), 8.04 (d, J=5.2 Hz, 1H), 7.72-7.64 (m, 1H), 7.44 (dd, J=20.9, 13.0 Hz, 2H), 7.37 (t, J=6.9 Hz, 1H), 7.26 (dt, J=27.6, 8.5 Hz, 3H), 5.93 (d, J=67.4 Hz, 1H), 3.16 (s, 2H), 2.25 (d, J=7.6 Hz, 2H), 1.73 (s, 2H), 1.41 (s, 9H) (25 non-exchangeable protons). ¹³C NMR (126 MHz, MeOD) δ 185.33, 177.11, 162.00 (d, J=3.5 Hz), 161.34, 159.95 (d, J=3.6 Hz), 156.76, 154.20 (d, J=249.8 Hz), 148.44, 136.63 (t, J=11.3 Hz), 135.14, 129.84, 127.65, 126.23 (d, J=13.3 Hz), 125.89 (d, J=4.6 Hz), 118.99 (t, J=15.9 Hz), 114.24 (d, J=3.7 Hz), 114.05 (d, J=3.5 Hz), 106.94, 41.67, 39.13, 32.27, 30.90, 25.62 (23 carbons due to symmetry). ¹⁹F NMR (471 MHz, MeOD) δ-108.60 (dt, J=9.3, 3.9 Hz), -127.42. HRMS(ESI): m/z calc. for C27H27F3N504S2 [M+H]⁺: 606.1451, found: 606.1448.

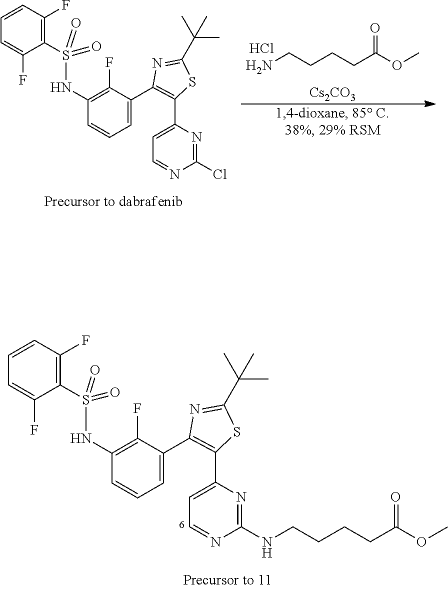

Methyl 5-((4-(2-(tert-butyl)-4-(3-((2,6-difluorophenyl)sulfonamido)-2-fluorophenyl)thiazol-5-yl)pyrimidin-2-yl)amino)pentanoate (precursor to 11). Prepared from the precursor to dabrafenib and methyl 5-aminopentanoate hydrochloride according to the general procedure to yield the precursor to 11 (43 mg, 38%) as a pale-yellow solid and recovered starting material (29%). ¹H NMR (500 MHz, CDCl3) δ 7.97-7.91 (m, 1H), 7.74-7.68 (m, 1H), 7.56-7.41 (m, 2H), 7.32 (ddt, J=7.9, 6.4, 1.3 Hz, 1H), 7.20 (td, J=7.9, 1.0 Hz, 1H), 7.01-6.94 (m, 2H), 6.07 (d, J=4.9 Hz, 1H), 5.50 (bs, 1H), 3.68 (d, J=0.9 Hz, 3H), 3.35 (s, 2H), 2.40-2.35 (m, 2H), 1.71 (h, J=6.9 Hz, 2H), 1.63 (q, J=7.2 Hz, 2H), 1.47 (d, J=0.9 Hz, 9H) (29 non-exchangeable protons). ¹³C NMR (151 MHz, CDCl3) δ 182.74, 173.98, 161.89, 160.64 (d, J=3.3 Hz), 158.92 (d, J=3.3 Hz), 158.70, 157.96, 150.98 (d, J=247.3 Hz), 145.66, 135.22 (t, J=11.0 Hz), 134.21, 128.16, 125.12 (d, J=4.3 Hz), 124.58, 124.49, 123.05, 116.92 (t, J=15.3 Hz), 113.26 (d, J=3.5 Hz), 113.11 (d, J=3.6 Hz), 106.28, 51.60, 40.88, 38.03, 33.69, 30.73, 28.99, 22.22 (27 carbons due to symmetry). ¹⁹F NMR (471 MHz, CDCl3) δ-106.85 (dt, J=9.9, 4.6 Hz), -130.25. HRMS(ESI): m/z calc. for C29H31F3N504S2 [M+H]⁺: 634.1764, found: 634.1768.

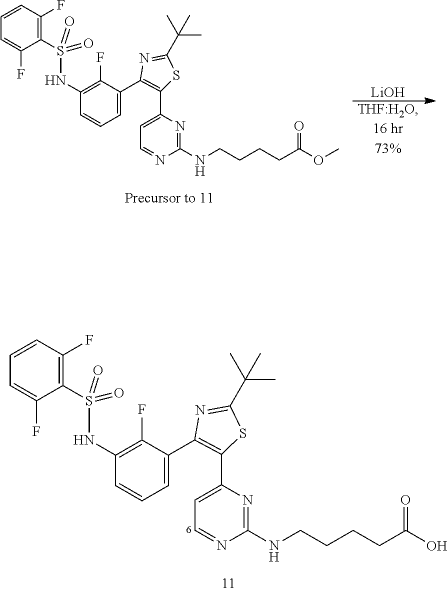 [SMILES: COC(=O)CCCCNc1nccc(-c2sc(C(C)(C)C)nc2-c2cccc(NS(=O)(=O)c3c(F)cccc3F)c2F)n1 ; "LiOH" ; "THF:H2O,16 hr73%" ; CC(C)(C)c1nc(-c2cccc(NS(=O)(=O)c3c(F)cccc3F)c2F)c(-c2ccnc(NCCCCC(=O)O)n2)s1]

5-((4-(2-(tert-butyl)-4-(3-((2,6-difluorophenyl)sulfonamido)-2-fluorophenyl)thiazol-5-yl)pyrimidin-2-yl)amino)pentanoic acid (11). Prepared from precursor to 11 according to a general hydrolysis procedure (trituration) to yield 11 (15 mg, 73%) as an off-white solid. ¹H NMR (500 MHz, MeOD) δ 8.02 (s, 1H), 7.62 (tt, J=8.4, 5.9 Hz, 1H), 7.51 (td, J=7.8, 1.8 Hz, 1H), 7.42 (td, J=7.0, 1.7 Hz, 1H), 7.30 (t, J=7.9 Hz, 1H), 7.10 (t, J=9.3 Hz, 2H), 6.47 (d, J=37.1 Hz, 1H), 3.34 (d, J=8.1 Hz, 2H), 2.35 (d, J=6.7 Hz, 2H), 1.77-1.57 (m, 4H), 1.49 (s, 9H) (25 non-exchangeable protons). ¹H NMR (500 MHz, DMSO) δ 10.88 (s, 1H), 8.08-8.03 (m, 1H), 7.68 (tt, J=8.4, 6.0 Hz, 2H), 7.47-7.40 (m, 1H), 7.40-7.35 (m, 1H), 7.29 (t, J=7.9 Hz, 1H), 7.24 (t, J=9.2 Hz, 2H), 5.96 (d, J=86.9 Hz, 1H), 3.15 (s, 1H), 2.24 (t, J=6.9 Hz, 2H), 1.56-1.46 (m, 5H), 1.41 (s, 9H) (27 non-exchangeable protons). ¹³C NMR (126 MHz, MeOD) δ 187.57, 177.19, 161.93 (d, J=3.8 Hz), 159.88 (d, J=3.7 Hz), 156.46, 154.19 (d, J=250.9 Hz), 151.22, 136.63 (t, J=11.1 Hz), 133.46, 129.77, 127.91, 126.45 (d, J=13.3 Hz), 126.13 (d, J=4.5 Hz), 125.28, 119.23, 114.28 (d, J=3.7 Hz), 114.10 (d, J=3.6 Hz), 107.05, 42.20, 39.47, 34.34, 30.83, 29.27, 23.14 (24 carbons due to symmetry). ¹⁹F NMR (471 MHz, CDCl3) δ-106.79--106.94 (m), -130.34. HRMS(ESI): m/z calc. for C28H29F3N504S2 [M+H]⁺: 620.1608, found: 620.1609.

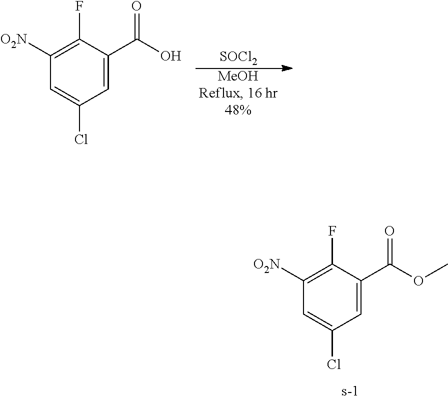

Methyl 5-chloro-2-fluoro-3-nitrobenzoate (s-1). In a round bottom flask, 5-chloro-2-fluoro-3-nitrobenzoic acid (5.3 g, 24.12 mmol, 60% purity from Oakwood) was dissolved in anhydrous methanol (75 mL) and cooled to 0° C. At 0° C., thionyl chloride (5.74 g, 48.24 mmol, 2 eq.) was added dropwise. The reaction was allowed to reach room temperature and then heated to reflux. After 16 hours, the reaction was removed from heat and the solvent was removed in vacuo. The residue was dissolved in ethyl acetate and washed with saturated aqueous sodium bicarbonate three times. The organic layer was then dried over anhydrous magnesium sulfate, filtered, and concentrated in vacuo. The resulting residue was purified via silica gel chromatography (slow gradient of 8 to 15% ethyl acetate in hexane) two to three times to afford s-1 (2.7 g, 48%) as a white solid. ¹H NMR (500 MHz, CDCl3) δ 8.76-7.96 (m, 2H), 3.99 (s, 3H). ¹³C NMR (126 MHz, CDCl3) δ 162.22 (d, J=3.8 Hz), 153.50 (d, J=278.4 Hz), 137.03, 129.77, 129.74, 129.73, 123.00 (d, J=11.5 Hz), 53.52. ¹⁹F NMR (471 MHz, CDCl3) δ-118.48. HRMS(ESI): m/z calc. for C8H6ClFNO4 [M+H]⁺: 233.9964, found: 233.9968.

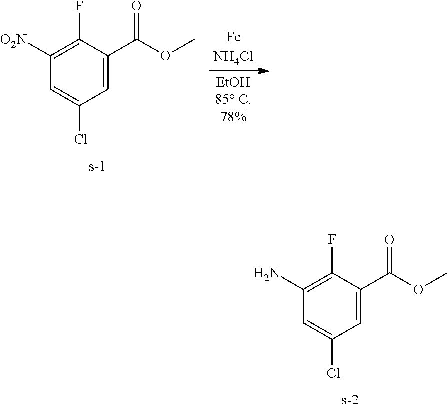

Methyl 3-amino-5-chloro-2-fluorobenzoate (s-2). In a round bottom flask, s-1 (2.75 g, 11.8 mmol), iron powder (2.63 g, 47.09 mmol, 4 eq.) and ammonium chloride (2.2 g, 41.2 mmol, 3.5 eq.) were suspended in ethanol (39.2 mL) and water (11.8 mL). The reaction was stirred at 85° C. for 2 hours. The reaction was cooled and then diluted in ethyl acetate and saturated aqueous sodium bicarbonate. The organic layer was extracted with ethyl acetate thrice, and the combined organics were washed with brine and dried over anhydrous magnesium sulfate, filtered, and concentrated in vacuo. The resulting residue was purified via silica gel chromatography (25-33% ethyl acetate in hexane) to afford s-2 (1.87 g, 78%) as a pale-yellow solid. ¹H NMR (500 MHz, CDCl3) δ 7.22-7.20 (m, 1H), 6.92-6.89 (m, 1H), 3.95 (d, J=3.6 Hz, 2H), 3.91 (s, 3H). ¹³C NMR (126 MHz, CDCl3) δ 164.15 (d, J=3.5 Hz), 149.39 (d, J=253.2 Hz), 136.98 (d, J=14.5 Hz), 129.23 (d, J=3.8 Hz), 119.95 (d, J=4.7 Hz), 119.72, 119.66, 52.64. ¹⁹F NMR (471 MHz, CDCl3) δ-135.52. HRMS(ESI): m/z calc. for C8H8ClFNO2 [M+H]⁺: 204.0222, found: 204.0224.

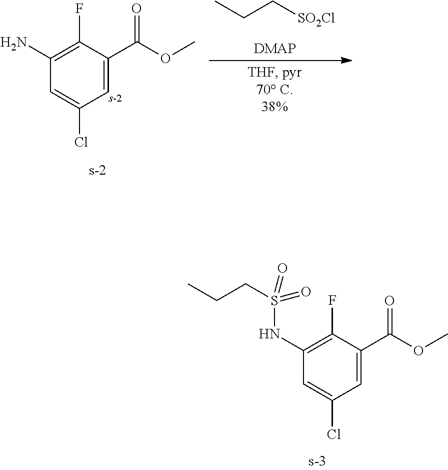

Methyl 5-chloro-2-fluoro-3-(propylsulfonamido)benzoate (s-3). In a round bottom flask, s-2 (250 mg, 1.130 mmol) and 4-dimethylaminopyridine (27.6 mg, 0.226 mmol, 0.2 eq.) were dissolved in anhydrous tetrahydrofuran (4.2 mL), and then pyridine (1.4 mL) was added. After cooling the reaction to 0° C., propane-1-sulfonyl chloride was added dropwise. After stirring at room temperature for 15 min, the vessel was warmed to 70° C. for 15 hr. The reaction was then diluted in ethyl acetate and 1 μM hydrochloric acid. The organic layer was extracted with ethyl acetate thrice, and the combined organics were washed with brine and dried over anhydrous magnesium sulfate, filtered, and concentrated in vacuo. The resulting residue was purified via silica gel chromatography (slow gradient 15-25% ethyl acetate in hexane). The combined fractions were further purified by trituration from dichloromethane and pentane to afford s-3 (115 mg, 38%) a pale-yellow solid. ¹H NMR (500 MHz, CDCl3) δ 7.80 (dd, J=6.3, 2.6 Hz, 1H), 7.68 (dd, J=5.8, 2.6 Hz, 1H), 6.60 (d, J=3.6 Hz, 1H), 3.95 (s, 3H), 3.18-3.06 (m, 2H), 1.95-1.79 (m, 2H), 1.06 (t, J=7.4 Hz, 3H). ¹³C NMR (126 MHz, CDCl3) δ 163.05 (d, J=3.6 Hz), 150.98 (d, J=257.2 Hz), 130.27 (d, J=4.3 Hz), 127.63 (d, J=13.6 Hz), 127.11, 125.40, 120.30 (d, J=10.2 Hz), 54.55, 53.05, 17.41, 12.96. ¹⁹F NMR (471 MHz, CDCl3) δ-129.91 (d, J=4.3 Hz). HRMS(ESI): m/z calc. for C11H12ClFNO4S [M−H]⁻: 308.0165, found: 308.0167.

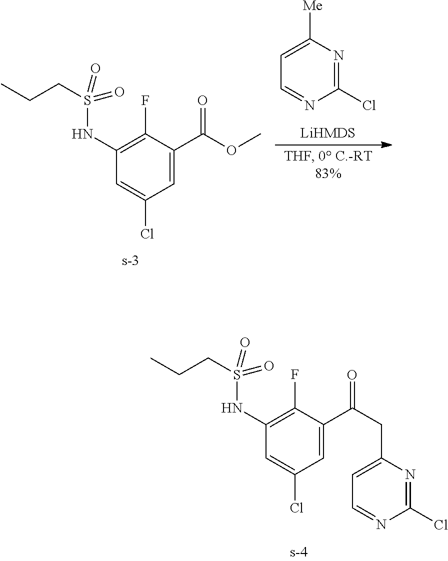 [SMILES: CCCS(=O)(=O)Nc1cc(Cl)cc(C(=O)OC)c1F ; Cc1ccnc(Cl)n1 ; CCCS(=O)(=O)Nc1cc(Cl)cc(C(=O)Cc2ccnc(Cl)n2)c1F]

N-(5-chloro-3-(2-(2-chloropyrimidin-4-yl)acetyl)-2-fluorophenyl)propane-1-sulfonamide (s-4). In a round bottom flask, s-3 (280 mg, 0.904 mmol) was dissolved in anhydrous tetrahydrofuran (6.45 mL) and cooled to 0° C. One molar solution of lithium bis(trimethylsilyl)amide in tetrahydrofuran (2.98 mL, 2.98 mmol, 3.3 eq.) was added dropwise, and then 2-chloro-4-methylpyrimidine (145.3 mg, 1.13 mmol, 1.25 eq.) dissolved in tetrahydrofuran (1.95 mL) was added dropwise. The reaction was warm to room temperature, and stirred for 1 hr. The reaction was then diluted in ethyl acetate and 1 μM hydrochloric acid. The organic layer was extracted with ethyl acetate thrice, and the combined organics were washed with brine and dried over anhydrous magnesium sulfate, filtered, and concentrated in vacuo. The resulting residue was purified via silica gel chromatography (slow gradient 15-25% ethyl acetate in hexane). The combined fractions were further purified by trituration from dichloromethane and pentane to afford s-4 (115 mg, 83%) a pale-yellow solid. ¹H NMR (500 MHz, CDCl3) δ 8.48 (d, J=5.4 Hz, 1H), 7.69 (dd, J=6.6, 2.6 Hz, 1H), 7.65 (dd, J=6.3, 2.6 Hz, 1H), 6.97 (d, J=5.4 Hz, 1H), 6.59 (d, J=3.6 Hz, 1H), 6.16 (s, 1H), 3.17-3.12 (m, 2H), 1.95-1.86 (m, 3H), 1.08 (t, J=7.5 Hz, 3H). (14 non-exchangeable protons). ¹³C NMR (126 MHz, CDCl3) δ 166.53, 161.08 (d, J=4.1 Hz), 158.87, 158.25, 149.41 (d, J=250.5 Hz), 130.63 (d, J=3.9 Hz), 127.03 (d, J=14.4 Hz), 124.63 (d, J=1.8 Hz), 124.40 (d, J=10.6 Hz), 122.89, 116.38, 99.33 (d, J=13.8 Hz), 54.41, 17.29, 12.86. ¹⁹F NMR (471 MHz, CDCl3) δ-131.40--131.53 (m). HRMS(ESI): m/z calc. for C15H15Cl2FN3O3S [M+H]⁺: 406.0190, found: 406.0185.

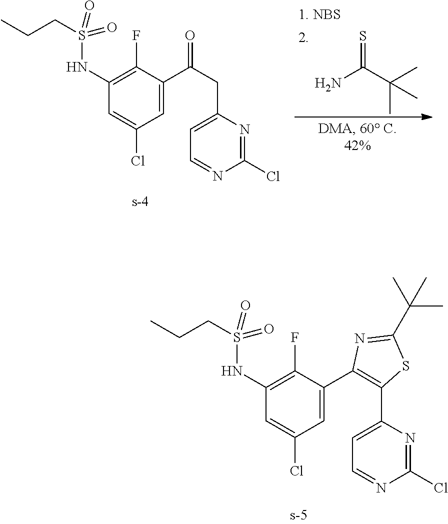

N-(3-(2-(tert-butyl)-5-(2-chloropyrimidin-4-yl)thiazol-4-yl)-5-chloro-2-fluorophenyl)propane-1-sulfonamide (s-5). In a vial with a stir bar, s-4 (232.8 mg, 0.577 mmol) was dissolved in anhydrous dimethyl acetamide (5.77 mL), and N-bromosuccinimide (113 mg, 0.635 mmol, 1.1 eq.) was added in one-portion. After stirring for 15 min, 2,2-dimethylpropanethioamide (71.05 mg, 1.05 eq.) was added in one-portion and stirred for another 15 min, and then the reaction was warmed to 60° C. After 15 hr, the reaction was diluted in water, and extracted with ethyl acetate three times. The combined organics were washed with brine and dried over anhydrous magnesium sulfate, filtered, and concentrated in vacuo, and residual dimethyl acetamide was blown off by a stream of nitrogen. The resulting residue was purified via silica gel chromatography (20-25% ethyl acetate in hexane) to afford s-5 (120.8 mg, 42%) as a pale-yellow solid. ¹H NMR (500 MHz, CDCl3) δ 8.46 (d, J=5.2 Hz, 1H), 7.70 (dd, J=6.7, 2.6 Hz, 1H), 7.38 (dd, J=5.7, 2.6 Hz, 1H), 6.99 (dd, J=5.3, 0.8 Hz, 1H), 6.46 (d, J=3.2 Hz, 1H), 3.16-3.10 (m, 2H), 1.91-1.83 (m, 2H), 1.51 (s, 9H), 1.06 (t, J=7.4 Hz, 3H). ¹³C NMR (126 MHz, CDCl3) (126 MHz, CDCl3) δ 185.14, 161.57, 160.79, 159.92, 148.85 (d, J=246.2 Hz), 146.31, 132.26, 130.88 (d, J=4.0 Hz), 127.12 (d, J=13.9 Hz), 126.79, 124.97 (d, J=15.4 Hz), 122.07, 115.44, 54.60, 38.53, 30.81, 17.42, 12.96. ¹⁹F NMR (471 MHz, CDCl3) δ-131.86 (d, J=4.0 Hz). HRMS(ESI): m/z calc. for C20H22Cl2FN402S2 [M+H]⁺: 503.0540, found: 503.0542.

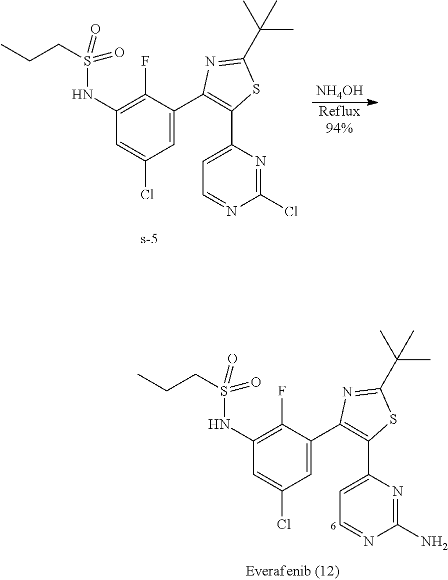

N-(3-(5-(2-aminopyrimidin-4-yl)-2-(tert-butyl)thiazol-4-yl)-5-chloro-2-fluorophenyl)propane-1-sulfonamide (everafenib, 12). In a vial with a stir bar, s-5 (144 mg, 0.286 mmol) was dissolved in concentrated ammonium hydroxide (3.5 mL). The vial was sealed and heated at 85° C. for 16 hr. The reaction was diluted with water, and neutralized with 1 μM hydrochloric acid, and the aqueous layer was extracted with ethyl acetate three times. The combined organics were dried over anhydrous magnesium sulfate, filtered, and concentrated in vacuo. The resulting residue was purified via trituration from dichloromethane, diethyl ether and pentane to afford everafenib (130 mg, 94%) as an off-white solid. ¹H NMR (500 MHz, CDCl3) δ 8.09 (d, J=5.2 Hz, 1H), 7.67 (dd, J=6.6, 2.6 Hz, 1H), 7.40 (dd, J=5.7, 2.6 Hz, 1H), 6.36 (d, J=5.2 Hz, 1H), 5.12 (s, 2H), 3.10-3.05 (m, 2H), 1.89-1.80 (m, 2H), 1.48 (s, 9H), 1.03 (t, J=7.4 Hz, 3H) (22 non-exchangeable protons). ¹H NMR (600 MHz, DMSO) δ 10.01 (s, 1H), 8.11 (d, J=5.2 Hz, 1H), 7.57 (dd, J=6.4, 2.6 Hz, 1H), 7.47 (dd, J=5.4, 2.7 Hz, 1H), 6.80 (s, 2H), 6.21 (d, J=5.1 Hz, 1H), 3.14-3.10 (m, 2H), 1.71-1.64 (m, 2H), 1.43 (s, 9H), 0.93 (t, J=7.4 Hz, 3H). ¹³C NMR (126 MHz, CDCl3) δ 183.44, 162.75, 159.40, 158.30, 149.67 (d, J=247.1 Hz), 144.52, 134.06, 130.47 (d, J=3.8 Hz), 127.63 (d, J=2.2 Hz), 126.64 (d, J=14.3 Hz), 125.34 (d, J=15.5 Hz), 123.60, 107.73, 54.35, 38.25, 30.82, 17.33, 12.98. ¹⁹F NMR (471 MHz, CDCl3) δ-130.61. HRMS(ESI): m/z calc. for C20H24CIFN5O2S2 [M+H]⁺: 484.1038, found: 484.1034.

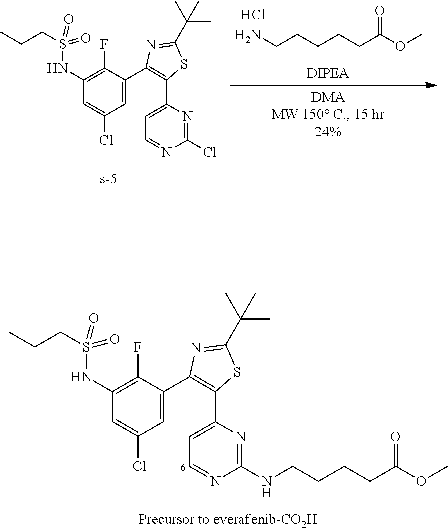

Methyl 5-((4-(2-(tert-butyl)-4-(5-chloro-2-fluoro-3-(propylsulfonamido)phenyl)thiazol-5-yl)pyrimidin-2-yl)amino)pentanoate (precursor to everafenib-CO₂H). In a microwave vial with a stir bar, s-5 (59.5 mg, 0.119 mmol) and methyl 5-aminopentanoate hydrochloride (80 mg, 0.476 mmol, 4 eq,) were dissolved in anhydrous dimethyl acetamide (1.19 mL). Diisopropyl ethylamine (166 L, 0.953 mmol, 8 eq.) was added to the mixture, and the vial was heated at 150° C. for 15 hr in a microwave reactor. The reaction was then diluted in ethyl acetate and 1 μM hydrochloric acid. The organic layer was extracted with ethyl acetate thrice, and the combined organics were washed with brine and dried over anhydrous magnesium sulfate, filtered, and concentrated in vacuo. The resulting residue was purified via silica gel chromatography (33-50% ethyl acetate in hexane) to afford the precursor to everafenib-CO₂H (16.5 mg, 24%) an off-white solid and 36 mg (63%) recovered starting material. ¹H NMR (500 MHz, CDCl3) δ 8.03 (s, 1H), 7.66 (dd, J=6.5, 2.6 Hz, 1H), 7.40 (dd, J=5.7, 2.6 Hz, 1H), 6.29 (d, J=5.2 Hz, 1H), 3.66 (s, 3H), 3.30 (s, 2H), 3.09-3.03 (m, 2H), 2.36 (t, J=7.3 Hz, 2H), 1.88-1.79 (m, 2H), 1.69 (p, J=7.2 Hz, 2H), 1.60 (q, J=6.7 Hz, 2H), 1.50 (s, 9H), 1.01 (t, J=7.5 Hz, 3H) (31 non-exchangeable protons). ¹H NMR (600 MHz, DMSO) δ 10.00 (s, 1H), 8.15 (d, J=5.2 Hz, 1H), 7.55 (s, 1H), 7.47-7.43 (m, 1H), 7.33 (t, J=5.9 Hz, 1H), 6.26 (d, J=91.4 Hz, 1H), 3.57 (s, 3H), 3.22 (d, J=21.4 Hz, 1H), 3.10 (t, J=7.7 Hz, 2H), 3.03 (s, 1H), 2.32 (t, J=7.3 Hz, 2H), 1.72-1.63 (m, 2H), 1.57-1.48 (m, 3H), 1.43 (s, 9H), 0.91 (t, J=7.4 Hz, 3H) (32 non-exchangeable protons). ¹³C NMR (126 MHz, CDCl3) δ 183.23, 174.13, 161.78, 158.97, 157.78, 149.55 (d, J=246.7 Hz), 144.65, 134.54, 130.41 (d, J=3.9 Hz), 127.35, 126.71 (d, J=14.1 Hz), 125.77, 122.80, 106.71, 54.34, 51.73, 41.00, 38.28, 33.78, 30.87, 29.03, 22.30, 17.36, 12.96 (24 carbons due to symmetry). ¹⁹F NMR (471 MHz, CDCl3) δ-131.09 (t, J=6.0 Hz). HRMS(ESI): m/z calc. for C26H34C1FN504S2 [M+H]⁺: 598.1719, found: 598.1718.

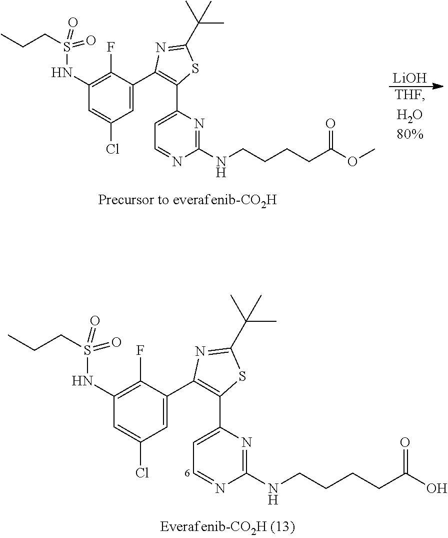

5-((4-(2-(tert-butyl)-4-(5-chloro-2-fluoro-3-(propylsulfonamido)phenyl)thiazol-5-yl)pyrimidin-2-yl)amino)pentanoic acid (everafenib-CO₂H, 13). Prepared from the precursor to everafenib-CO₂H according to a general hydrolysis procedure (trituration) to yield everafenib-CO₂H (38.3 mg, 80%) as an off-white solid. ¹H NMR (500 MHz, MeOD) δ 8.11 (d, J=5.9 Hz, 1H), 7.66 (dd, J=6.5, 2.7 Hz, 1H), 7.40 (dd, J=5.5, 2.6 Hz, 1H), 6.55 (d, J=6.0 Hz, 1H), 3.26 (s, 2H), 3.15-3.08 (m, 2H), 2.35 (t, J=7.0 Hz, 2H), 1.88-1.76 (m, 2H), 1.69-1.56 (m, 4H), 1.51 (d, J=2.3 Hz, 9H), 1.02 (t, J=7.4 Hz, 3H) (28 non-exchangeable protons). ¹H NMR (500 MHz, DMSO) δ 10.01 (s, 1H), 8.18 (d, J=5.4 Hz, 1H), 7.65 (s, 1H), 7.58-7.54 (m, 1H), 7.46 (dd, J=5.4, 2.7 Hz, 1H), 6.33 (d, J=84.8 Hz, 1H), 3.31-3.14 (m, 1H), 3.15-3.09 (m, 2H), 3.05 (s, 1H), 2.23 (t, J=6.9 Hz, 2H), 1.73-1.64 (m, 2H), 1.48 (d, J=17.6 Hz, 4H), 1.44 (s, 9H), 0.92 (t, J=7.4 Hz, 3H) (30 non-exchangeable protons). ¹³C NMR (126 MHz, MeOD) δ 186.69, 177.25, 163.81, 158.42, 153.06, 151.88 (d, J=247.8 Hz), 148.89, 134.37, 130.83 (d, J=4.1 Hz), 128.98 (d, J=14.6 Hz), 127.89, 126.85, 125.94, 107.43, 55.40, 42.05, 39.43, 34.42, 30.89, 29.43, 23.19, 18.34, 13.12 (23 carbons due to symmetry). ¹⁹F NMR (471 MHz, MeOD) δ-126.25. HRMS(ESI): m/z calc. for C25H32ClFN5O4S2 [M+H]⁺: 584.1563, found: 585.1564.

### Example 4. Additional Compounds of the Disclosed Technology that can be Prepared According to the Synthetic Methods Described Herein

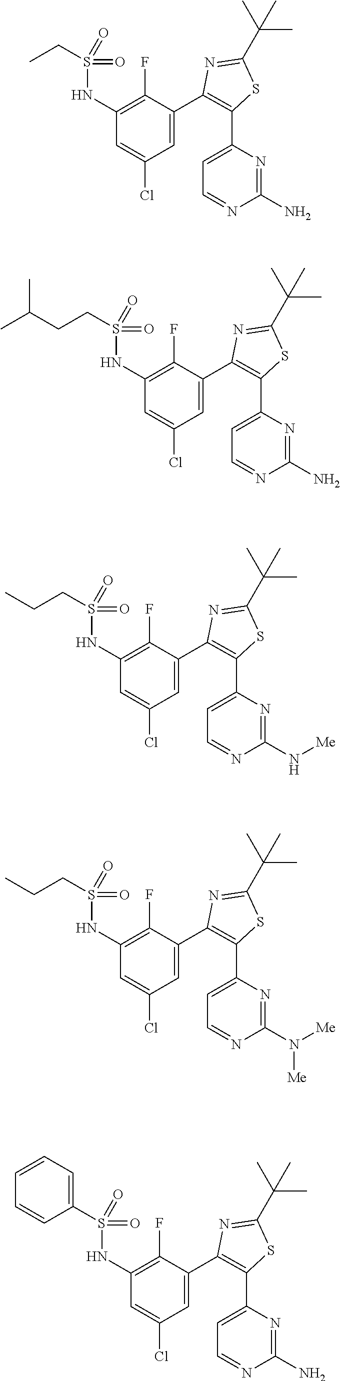

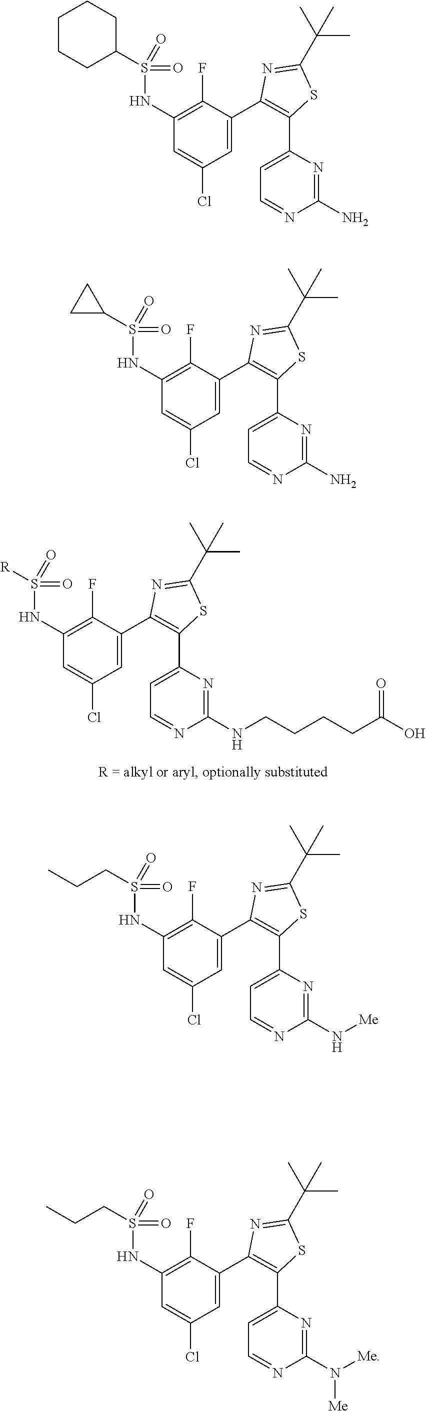

### Example 5. Materials and Methods for Biological Experiments

Cell Culture and reagents. All cell lines were grown in a 37° C., 5% CO₂, humidified environment, in media containing 1% penicillin/streptomycin. Cell culture conditions are as follows: MDCK cells transfected with MDR1 (or ABCB1) were obtained from the Netherlands Cancer Institute (Amsterdam, The Netherlands) and maintained in DMEM with 10% fetal bovine serum (FBS, Gemini). Traditional cell lines A375, CHL-1, B16F10, and U118MG were grown in DMEM with 10% FBS, SK-MEL-28, U87 and T98G in EMEM with 10% FBS, AM-38 in EMEM with 20% HI-FBS (Gibco), HT29 in McCoy's 5A with 10% FBS, HCT116 in RPMI with 10% FBS. Compounds were dissolved in DMSO (1% final concentration, Sigma-Aldrich) for cell culture studies.

P-gp transwell bidirectional transport assays. Unless noted otherwise, the transport assay was performed in triplicate in batches of 16 or 24 test compounds, with each batch containing quinidine as a substrate control and propranolol as a non-substrate control. MDR1-MDCK cells were plated at a density of 300,000 cells/cm² onto 96-well Falcon™ Multiwell Insert System with 1 m pore polycarbonate filters with angled-bottom receiver plates (Coming) and were fed with the cell growth medium on day 2.

The donor solution was prepared by adding 1 mM DMSO stocks of compounds to make the final concentration 10 M, and the receiver solution was prepared with equivalent volume of DMSO to make 1% DMSO in final assay chamber volumes.

After 80-96 hours since seeding, the growth media on both sides was aspirated. To the transwell plate (apical side), 50 μL of receiver and donor solutions prepared on a 96-well plate were added, and it was placed on top of a separate angled-bottom 96-well plate (basolateral side) containing 260 μL of receiver and donor solutions.

Following 90 minutes of incubation at 37° C. with shaking at 90 rpm, 20 μL aliquot of samples were taken from both the donor and receiver chambers, and the apical and basolateral sides. Aliquots of 4 test compounds were pooled for each category: apical-donor, apical-receiver, basolateral-donor, basolateral-receiver. To measure CO and mass balance, initial donor samples from both apical and basolateral side were set aside from the assay plate. Acetonitrile twice the volume of pooled samples was added and mixed thoroughly, and precipitated proteins were removed by centrifugation at 20,000 r.c.f for 10 minutes.

To ascertain the MDCK monolayer integrity, the solution on apical side contained 100 M Lucifer Yellow (Millipore-Sigma). After 90 minutes of compound transport, Lucifer Yellow concentration in the basal well was measured using SpectraMax M3 (Molecular Devices) set to an excitation wavelength of 430 nm and an emission wavelength of 540 nm. For each replicate, a standard curve was generated using the Lucifer Yellow solution used in the particular replicate and Papp A-B was calculated, and samples with Papp A-B<30 nm/s were further analyzed.

Samples were analyzed with the 5500 QTRAP LC/MS/MS system (AB Sciex) with a 1200 series HPLC system (Agilent Technologies) including a degasser, an autosampler, and a binary pump. The liquid chromatography separation was performed on an Agilent SB-Aq column (4.6×50 mm, 5 μm) (Agilent Technologies) with mobile phase A (0.1% formic acid in water) and mobile phase B (0.1% formic acid in acetonitrile). The flow rate was 0.3 ml min⁻¹. The linear gradient was optimized for each compound. The autosampler was set at 5° C. The injection volume was 15 μL. Mass spectra were acquired with both positive electrospray ionization at the ion spray voltage of 5,500 V and negative electrospray ionization at the ion spray voltage of −4,500 V. The source temperature was 450° C. The curtain gas, ion source gas 1, and ion source gas 2 were 33, 50 and 65, respectively. Multiple reaction monitoring was used to quantify metabolites.

Power analysis was not used to determine the number of replicates. Error bars represent the standard error of the mean of three or greater biological replicates. All compounds evaluated in biological assays were >95% pure.

Efflux data calculations. Apparent permeability (Papp) was calculated using Equation 1, efflux ratio (ER) using Equation 2, and mass balance using Equation 3.

$${P}_{app} = \frac{1}{S \cdot {C}_{0}} \cdot ( \frac{dQ}{dt} ) \qquad Eq . 1$$

Where S represents the surface area of the transwell (0.0802 cm²), CO the initial concentration, dQ the change in concentration and dt incubation time.

$$ER = \frac{{P}_{app} ( B \to A )}{{P}_{app} ( A \to B )} \qquad Eq . 2$$

$$MB = \frac{{C}_{A} {V}_{A} + {C}_{B} {V}_{B}}{{C}_{0} {V}_{0}} \qquad Eq . 3$$

Where CA represents measured concentration in the apical well, VA, apical chamber volume (50 μL), CB represents measured concentration in the basolateral well, and VB, basolateral chamber volume (260 μL).

Efflux inhibitor co-administration. 5 mM of elacridar (Millipore-Sigma, WI) in DMSO was added to the assay media to make 0.03% DMSO and then the test compounds were added to the media containing elacridar, to make final donor solution with 10 μM test compound, 2 μM elacridar and 1.03% DMSO. The receiver solution contained equivalent DMSO concentration. The reminder of assay conditions was kept the same as described above, and the lack of cytotoxicity by increased DMSO was confirmed by Lucifer Yellow.

Calcein-AMP-gp inhibition assay. On a 96-well flat clear bottom black microplates (Coming 3904), 70,000 cells of MDR1-MDCK were seeded in 200 μL volume and were changed with fresh growth media 24 hr after seeding. Experiments were performed 72 hours after seeding. Compounds (2× of final concentrations) were prepared in transport buffer (Hanks' Balanced Salt Solution supplemented with 0.33 mM sodium pyruvate) to make 1% DMSO. Growth media was aspirated, and the monolayer was washed with the transport buffer three times. The compound containing transport buffer (50 μL) was then incubated with the monolayer for 15 min. Calcein-AM (Invitrogen C1430) was dissolved in DMSO to make 1 mM, and further diluted in the transport buffer to 10 μM (2× of final, 1% DMSO), and protected from light. After the compound incubation, 50 μL of the calcein-AM solution was added, and cells were immediately read in the plate reader warmed to 37° C. The plate was read kinetically for 45 min at excitation wavelengths and emission wavelengths of 485 nm and 530 nm, respectively using SpectraMax M3 (Molecular Devices). In each plate, 1 μM elacridar was included to set 100% inhibition. % inhibition was calculated as (treated-background) divided by (elacridar-background)×100.

Blood-brain barrier penetrance P-gp in vivo assays. All experimental procedures were reviewed and approved by the University of Illinois Institutional Animal Care and Use Committee (Protocol number: 19191). CD-1 mice were purchased from Charles River and acclimated for 4-7 days before use. Mice were administered elacridar or its vehicle at 2.5 mg/kg via lateral tail vein injection. Thirty-minutes post injection, mice were then treated with compounds at 25 mg/kg intravenously now in the other tail vein. At each time point, mice were sacrificed and blood was collected by lacerating the right auricle with iris scissors. An 18-gauge angiocatheter was inserted through the left ventricle, and all residual circulatory volume was removed by perfusing 0.9% saline solution via an analog peristaltic pump. Brains were harvested from the cranial vault and flash frozen. Blood samples were centrifuged at 13,000 ref for 10 minutes and the supernatant serum was stored at −80° C. until analysis. Brain samples were homogenized in ice-cold methanol and centrifuged first at 2000 ref for three minutes and then 13,000 ref for ten minutes and supernatant and tissue debris were separated and stored at −80° C. The resultant supernatant and serum were analyzed by LC-MS/MS to determine compound concentrations. Compounds were formulated as the following: elacridar in 12% DMSO, 12% propylene glycol 76% 5% HPβCD, quinidine in 1% DMSO in 5% HPβCD, propranolol (given at 12.5 mg/kg) in 0.5% DMSO in 5% HPβCD, levofloxacin and levofloxacin-ME in 15% HPβCD, 10 in 10% HPβCD, tiagabine and cilomilast in 10% DMSO in 10% HPβCD, tianeptine in 10% DMSO in PBS, and encorafenib, dabrafenib, everafenib, and everafenib-CO₂H in 10% DMSO 40% PEG400 50% 10% HPβCD, and PLX4720 in 15% DMSO 35% PEG400 50% 15 HPβCD. All compounds were administered at 5 mL/kg.

Cell viability assays. Cells were harvested, seeded in a 96-well plate and allowed to adhere overnight. Compound was added to each well in DMSO (1% final concentration). Cells were incubated for 72 hours before viability was assessed by the Alamar Blue Assay. Raptinal (50 M) was used as a dead control.

Immunoblotting. Cells were lysed using RIPA buffer containing phosphatase (BioVision) and protease inhibitor cocktail (Calbiochem). Protein concentration was determined using the BCA assay (Pierce). Cell lysates containing 10 g of protein were loaded into each lane of 4% to 20% gradient gels (Bio-Rad) for SDS-PAGE. Proteins were transferred onto PDVF membrane for Western blot analysis. Blots were blocked with BSA for one hour followed by incubation with primary antibody overnight (dilution according to manufacturer's protocols), and then secondary antibody was incubated for one hour. Blots were then imaged with a ChemiDoc Touch (Bio-Rad) after incubation with SuperSignal West Pico Solution (ThermoFischer) following manufacturer's protocols. All antibodies were purchased from Cell Signaling Technology: p-ERK (9101S), ERK (4695S), p-MEK (9121S), MEK (8727S), vinculin (13901S) Rabbit IgG-HRP (7074), p-actin HRP (5125).

In vivo efficacy model. All experimental procedures were reviewed and approved by the University of Illinois Institutional Animal Care and Use Committee (Protocol number: 21155). Human melanoma A375 cells were intracranially implanted in 7-week-old female athymic nude mice (50,000 cells/mouse). Cells were tested to be free of pathogens prior to inoculation. Five days after implantation of the tumor cells, mice were treated with vehicle or 50 mg/kg of dabrafenib or everafenib (formulated in 10% DMSO 40% PEG400, 50% 15% HPβCD) intraperitoneally once-per-day for 5 days. After two days off, another five daily doses were administered, total of ten treatments in the model. Dabrafenib and everafenib were dissolved fresh at 5 mg/mL for each dose. Mice were observed daily for any signs of pain and distress (deterioration, neurotoxicity or movement disorders) according to the protocol.

### Example 6. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| | mg/tablet |
|---|---|
| (i) Tablet 1 | |
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | |
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |
| | mg/mL |
| (iv) Injection 1 (1 mg/mL) | |
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/mL) | |
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |
| (vi) Aerosol | mg/can |
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |
| | wt. % |
| (vii) Topical Gel 1 | |
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |
| (viii) Topical Gel 2 | |
| ‘Compound X’ | 5% |
| Methylcellulose | 2% |
| Methyl paraben | 0.2% |
| Propyl paraben | 0.02% |
| Purified water | q.s. to 100 g |
| (ix) Topical Ointment | |
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |
| (x) Topical Cream 1 | |
| ‘Compound X’ | 5% |
| White bees wax | 10% |
| Liquid paraffin | 30% |
| Benzyl alcohol | 5% |
| Purified water | q.s. to 100 g |
| (xi) Topical Cream 2 | |
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound of Formula I: 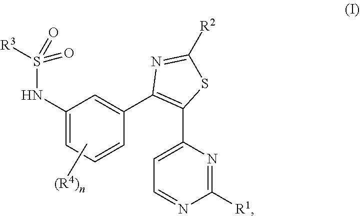
or a salt thereof;
wherein
R¹ is —NR^{a}R^{b}, —(C₁-C₆)alkyl-J¹, or —(C₃-C₆)cycloalkyl-J²;
R^{a} is H, —(C₁-C₆)alkyl-J³, —(C₃-C₆)cycloalkyl-J⁴, or phenyl-J⁵;
R^{b} is H, —(C₁-C₆)alkyl-J³, —(C₃-C₆)cycloalkyl-J⁴, or phenyl-J⁵;
J¹, J², J³, J⁴, and J⁵ are each independently CO₂H or tetrazol-2-yl;
R² is —(C₁-C₆)alkyl or —(C₄-C₆)cycloalkyl;
R³ is —(C₂-C₆)alkyl, —(C₃-C₆)cycloalkyl, or phenyl(R^{c})ₘ;
each R^{c} is independently halo or —(C₁-C₆)alkyl;
m is 0, 1,2,3,4, or 5;
each R⁴ is independently halo or —(C₁-C₆)alkyl; and
n is 2, 1, 3, 4, or 0;
wherein each alkyl moiety is independently branched or unbranched, and optionally substituted;
provided that R³ is not 2,6-difluorophenyl when R¹ is NH₂, R² is tert-butyl, R⁴ is 2-fluoro, and n is 1; R³ is not 2,5-difluorophenyl when R¹ is NH₂, R² is tert-butyl, R⁴ is 5-chloro and 2-fluoro, and n is 2; and
R³ is not n-propyl when R¹ is NH₂, R² is tert-butyl, R⁴ is 2,5-chloro, and n is 2.

2. The compound of claim 1 wherein R¹ is —NHR^{b} or —(C₁-C₆)alkyl-J¹.

3. The compound of claim 1 wherein R¹ is: NH₂,

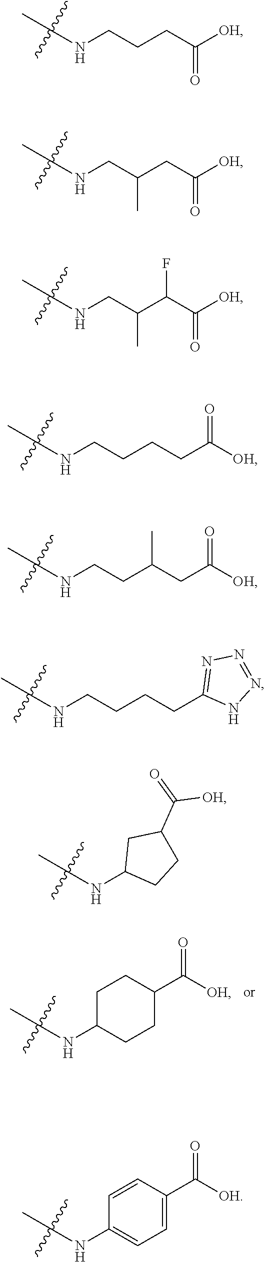

4. The compound of claim 1 wherein R¹ is:

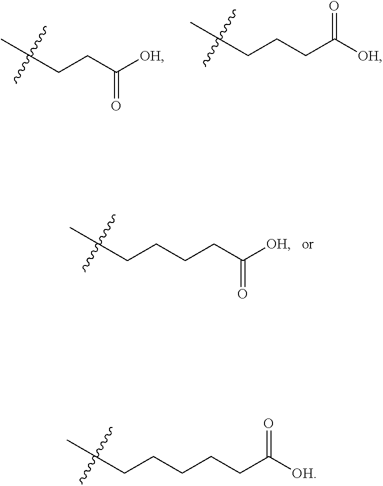 [SMILES: *CCCC(=O)O ; *CCC(=O)O ; *CCCCC(=O)O ; *CCCCCC(=O)O]

5. The compound of claim 1 wherein R² is tert-butyl or —C(CH₃)₂CO₂H.

6. The compound of claim 1 wherein R³ is: propyl, butyl, pentyl,

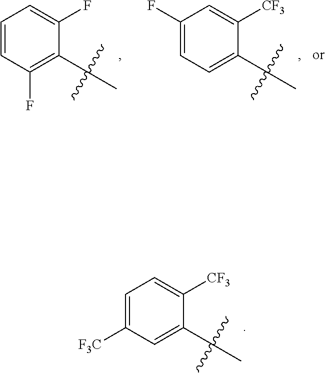 [SMILES: *c1c(F)cccc1F ; *c1ccc(F)cc1C(F)(F)F ; *c1cc(C(F)(F)F)ccc1C(F)(F)F]

7. The compound of claim 1 wherein one R⁴ is fluoro, another R⁴ is chloro, and n is 2.

8. The compound of claim 1 represented by Formula II: 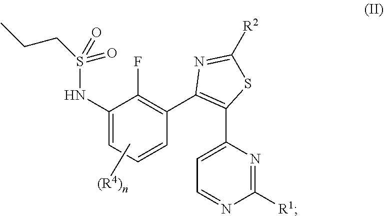
wherein n is 0, 1, 2, or 3.

9. The compound of claim 1 represented by Formula III:

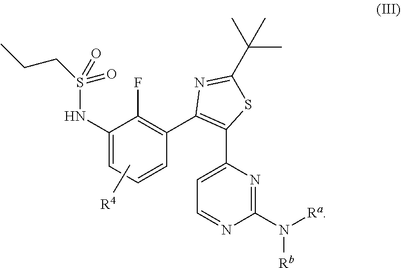

10. The compound of claim 1 represented by Formula IV: 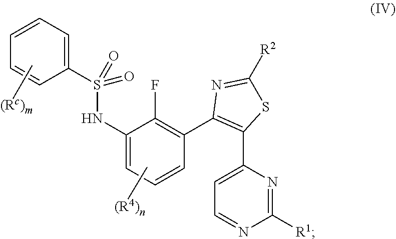
wherein n is 0, 1, 2, or 3.

11. The compound of claim 1 represented by Formula V 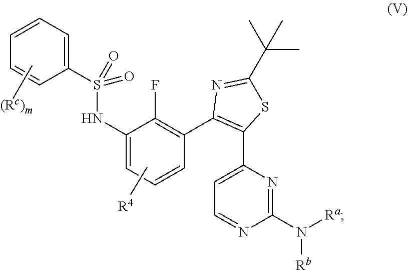
wherein R^{a} is H.

12. The compound of claim 9 wherein R^{a} is H; and R^{b} is: H,

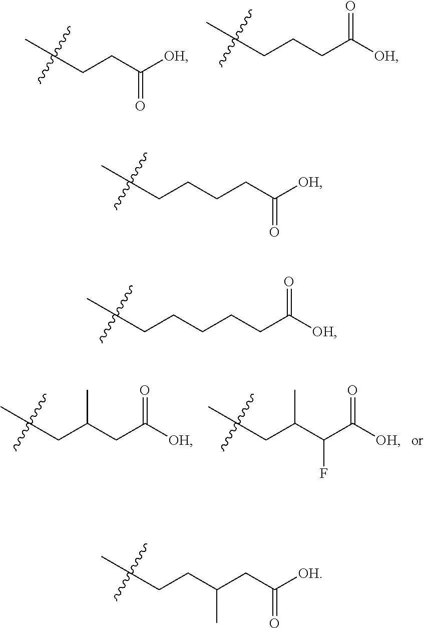

13. The compound of claim 1 wherein R¹ is —NH(C₁-C₆)alkyl-CO₂H or —(C₁-C₆)alkyl-CO₂H.

14. The compound of claim 1 wherein the compound is:

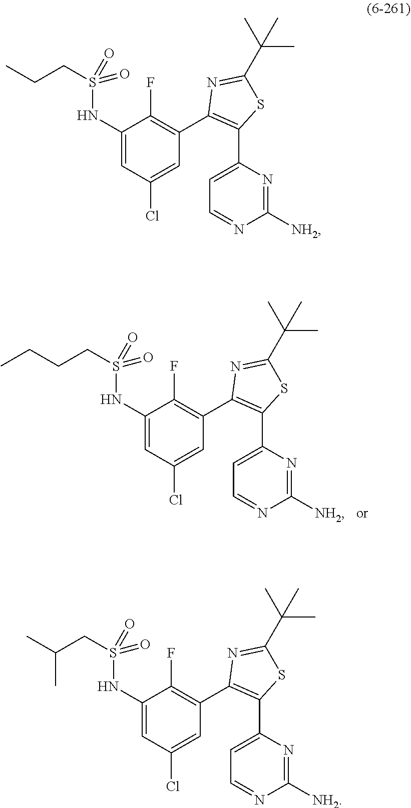 [SMILES: CCCS(=O)(=O)Nc1cc(Cl)cc(-c2nc(C(C)(C)C)sc2-c2ccnc(N)n2)c1F ; CCCCS(=O)(=O)Nc1cc(Cl)cc(-c2nc(C(C)(C)C)sc2-c2ccnc(N)n2)c1F ; "or" ; CC(C)CS(=O)(=O)Nc1cc(Cl)cc(-c2nc(C(C)(C)C)sc2-c2ccnc(N)n2)c1F]

15. The compound of claim 1 wherein the compound is:

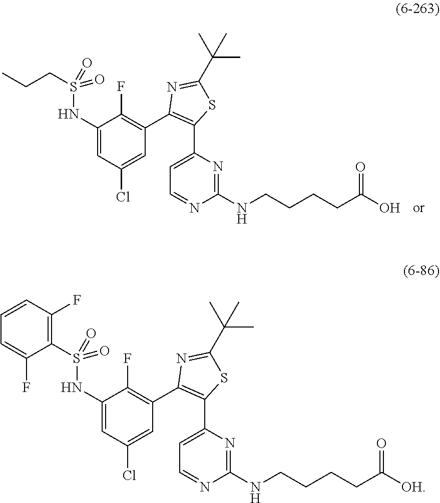 [SMILES: CCCS(=O)(=O)Nc1cc(Cl)cc(-c2nc(C(C)(C)C)sc2-c2ccnc(NCCCCC(=O)O)n2)c1F ; "or" ; CC(C)(C)c1nc(-c2cc(Cl)cc(NS(=O)(=O)c3c(F)cccc3F)c2F)c(-c2ccnc(NCCCCC(=O)O)n2)s1]

16. The compound of claim 1 wherein the compound is:
4-((4-(2-(tert-butyl)-4-(3-((2,6-difluorophenyl)sulfonamido)-2-fluorophenyl)thiazol-5-yl)pyrimidin-2-yl)amino)benzoic acid (6-77),
(1s,4s)-4-((4-(2-(tert-butyl)-4-(3-((2,6-difluorophenyl)sulfonamido)-2-fluorophenyl)thiazol-5-yl)pyrimidin-2-yl)amino)cyclohexane-1-carboxylic acid (6-85),
(1r,4r)-4-((4-(2-(tert-butyl)-4-(3-((2,6-difluorophenyl)sulfonamido)-2-fluorophenyl)thiazol-5-yl)pyrimidin-2-yl)amino)cyclohexane-1-carboxylic acid (6-89),
(1S,3R)-3-((4-(2-(tert-butyl)-4-(3-((2,6-difluorophenyl)sulfonamido)-2-fluorophenyl)thiazol-5-yl)pyrimidin-2-yl)amino)cyclopentane-1-carboxylic acid (6-91),
4-((4-(2-(tert-butyl)-4-(3-((2,6-difluorophenyl)sulfonamido)-2-fluorophenyl)thiazol-5-yl)pyrimidin-2-yl)amino)butanoic acid (6-86),
5-((4-(2-(tert-butyl)-4-(3-((2,6-difluorophenyl)sulfonamido)-2-fluorophenyl)thiazol-5-yl)pyrimidin-2-yl)amino)pentanoic acid (6-83),
N-(3-(5-(2-((4-(1H-tetrazol-5-yl)butyl)amino)pyrimidin-4-yl)-2-(tert-butyl)thiazol-4-yl)-2-fluorophenyl)-2,6-difluorobenzenesulfonamide (6-121),
3-(4-(2-(tert-butyl)-4-(3-((2,6-difluorophenyl)sulfonamido)-2-fluorophenyl)thiazol-5-yl)pyrimidin-2-yl)propanoic acid (6-97),
4-(4-(2-(tert-butyl)-4-(3-((2,6-difluorophenyl)sulfonamido)-2-fluorophenyl)thiazol-5-yl)pyrimidin-2-yl)butanoic acid (6-191),
5-(4-(2-(tert-butyl)-4-(3-((2,6-difluorophenyl)sulfonamido)-2-fluorophenyl)thiazol-5-yl)pyrimidin-2-yl)pentanoic acid (6-131),
6-(4-(2-(tert-butyl)-4-(3-((2,6-difluorophenyl)sulfonamido)-2-fluorophenyl)thiazol-5-yl)pyrimidin-2-yl)hexanoic acid (6-193),
5-((4-(2-(tert-butyl)-4-(2-fluoro-3-((4-fluoro-2-(trifluoromethyl)phenyl)sulfonamido)phenyl)thiazol-5-yl)pyrimidin-2-yl)amino)pentanoic acid (6-181),
N-(3-(5-(2-aminopyrimidin-4-yl)-2-(tert-butyl)thiazol-4-yl)-2-fluorophenyl)-4-fluoro-2-(trifluoromethyl)benzenesulfonamide (6-166),
N-(3-(5-(2-aminopyrimidin-4-yl)-2-(tert-butyl)thiazol-4-yl)-2,4-difluorophenyl)-4-fluoro-2-(trifluoromethyl)benzenesulfonamide (6-167),
5-((4-(2-(tert-butyl)-4-(2,6-difluoro-3-((4-fluoro-2-(trifluoromethyl)phenyl)sulfonamido)phenyl)thiazol-5-yl)pyrimidin-2-yl)amino)pentanoic acid (6-179),
N-(3-(5-(2-aminopyrimidin-4-yl)-2-(tert-butyl)thiazol-4-yl)-2-fluorophenyl)-2,5-bis(trifluoromethyl)benzenesulfonamide (6-163),
5-((4-(4-(3-((2,5-bis(trifluoromethyl)phenyl)sulfonamido)-2-fluorophenyl)-2-(tert-butyl)thiazol-5-yl)pyrimidin-2-yl)amino)pentanoic acid (6-173),
N-(3-(5-(2-aminopyrimidin-4-yl)-2-(tert-butyl)thiazol-4-yl)-2,4-difluorophenyl)-2,5-bis(trifluoromethyl)benzenesulfonamide (6-145),
5-((4-(4-(3-((2,5-bis(trifluoromethyl)phenyl)sulfonamido)-2,6-difluorophenyl)-2-(tert-butyl)thiazol-5-yl)pyrimidin-2-yl)amino)pentanoic acid (6-150),
N-(3-(5-(2-aminopyrimidin-4-yl)-2-(tert-butyl)thiazol-4-yl)-5-chloro-2-fluorophenyl)-5-fluoro-2-methylbenzenesulfonamide (6-244),
4-((4-(2-(tert-butyl)-4-(5-chloro-2-fluoro-3-((5-fluoro-2-methylphenyl)sulfonamido)phenyl)thiazol-5-yl)pyrimidin-2-yl)amino)-3-methylbutanoic acid (6-251),
5-((4-(2-(tert-butyl)-4-(5-chloro-2-fluoro-3-((5-fluoro-2-methylphenyl)sulfonamido)phenyl)thiazol-5-yl)pyrimidin-2-yl)amino)pentanoic acid (6-249),
N-(3-(5-(2-aminopyrimidin-4-yl)-2-(tert-butyl)thiazol-4-yl)-5-chloro-2-fluorophenyl)propane-1-sulfonamide (6-261),
5-((4-(2-(tert-butyl)-4-(5-chloro-2-fluoro-3-(propylsulfonamido)phenyl)thiazol-5-yl)pyrimidin-2-yl)amino)pentanoic acid (6-263),
4-((4-(2-(tert-butyl)-4-(5-chloro-2-fluoro-3-(propylsulfonamido)phenyl)thiazol-5-yl)pyrimidin-2-yl)amino)-3-methylbutanoic acid (6-265),
4-((4-(2-(tert-butyl)-4-(5-chloro-2-fluoro-3-(propylsulfonamido)phenyl)thiazol-5-yl)pyrimidin-2-yl)amino)-2-fluoro-3-methylbutanoic acid (8-41),
(R)-5-((4-(2-(tert-butyl)-4-(5-chloro-2-fluoro-3-(propylsulfonamido)phenyl)thiazol-5-yl)pyrimidin-2-yl)amino)-3-methylpentanoic acid (8-43),
N-(3-(5-(2-aminopyrimidin-4-yl)-2-(tert-butyl)thiazol-4-yl)-5-chloro-2-fluorophenyl)butane-1-sulfonamide, or
N-(3-(5-(2-aminopyrimidin-4-yl)-2-(tert-butyl)thiazol-4-yl)-5-chloro-2-fluorophenyl)-2-methylpropane-1-sulfonamide.

17. A composition comprising a compound of claim 1 and a pharmaceutically acceptable excipient.

18. A method for treatment of cancer comprising, administering to a subject in need of cancer treatment a therapeutically effective amount of a compound of Formula I: 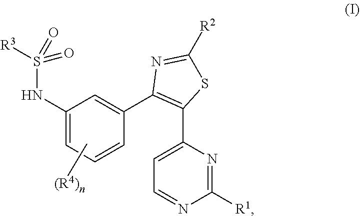
or a salt thereof;
wherein
R¹ is —NR^{a}R^{b}, —(C₁-C₆)alkyl-J¹, or —(C₃-C₆)cycloalkyl-J²;
R^{a} is H, —(C₁-C₆)alkyl-J³, —(C₃-C₆)cycloalkyl-J⁴, or phenyl-J⁵;
R is H, —(C₁-C₆)alkyl-J³, —(C₃-C₆)cycloalkyl-J⁴, or phenyl-J⁵;
J¹, J, J⁴, and J⁵ are each independently CO₂H or tetrazol-2-yl;
R² is —(C₁-C₆)alkyl or —(C₄-C₆)cycloalkyl;
R³ is —(C₂-C₆)alkyl, —(C₃-C₆)cycloalkyl, or phenyl(R^{c})ₘ;
each R^{c} is independently halo or —(C₁-C₆)alkyl;
m is 0, 1,2,3,4, or 5;
each R⁴ is independently halo or —(C₁-C₆)alkyl; and
n is 2, 1, 3, 4, or 0;
wherein each alkyl moiety is independently branched or unbranched, and optionally substituted; and
wherein the compound has a permeability glycoprotein (P-gp) efflux ratio of about 2 or less.

19. The method of claim 18 wherein the compound has a P-gp efflux ratio of 1.0±0.75.

20. The method of claim 18 wherein the compound has a brain to serum ratio of about 0.25 or more.

21. (canceled)

22. (canceled)

23. (canceled)

24. (canceled)
